(19) United States
(12) Patent Application Publication
(10) Pub. No.: US 2025/0061986 A1
(43) Pub. Date: Feb. 20, 2025

(54) FRACTIONAL DYNAMICS FOSTER DEEP LEARNING OF MEDICAL CONDITION PREDICTION

(71) Applicants: University of Southern California, Los Angeles, CA (US); Politehnica University Timisoara, Timisoara (RO); Victor Babes University of Medicine and Pharmacy Timisoara, Timisoara (RO); COPD Foundation, Inc., Miami, FL (US)

(72) Inventors: Paul Bogdan, Los Angeles, CA (US); Mingxi Cheng, Los Angeles, CA (US); Gaurav Gupta, San Jose, CA (US); Andrei Lihu, Timisoara (RO); David Mannino, Miami, FL (US); Stefan Mihaicuta, Timisoara (RO); Mihai Udrescu, Timisoara (RO); Lucretia Udrescu, Timisoara (RO); Chenzhong Yin, Los Angeles, CA (US)

(21) Appl. No.: 18/235,218

(22) Filed: Aug. 17, 2023

(30) Foreign Application Priority Data
Aug. 16, 2023 (RO) A 2023 00458

(51) Int. Cl. G16H 10/60; G16H 50/20

(52) U.S. Cl. CPC G16H 10/60; G16H 50/20

(57) ABSTRACT

Applicant discloses herein relate to systems, methods, apparatuses, and non-transitory computer readable media for generating physiological signals datasets, analyzing physiological signals in the physiological signals datasets, extract fractional dynamics signatures specific to Chronic Obstructive Pulmonary Disease (COPD) medical records, and identifying, using a deep neural network (DNN), a COPD stage.

### FEDERALLY SPONSORED RESEARCH OR DEVELOPMENT

This invention was made with government support under Grant No(s). N66001-17-1-4044, CPS/CNS-1453860, CNS-1932620, CCF-1837131, and MCB-1936775, awarded by the Defense Advanced Research Projects Agency (DARPA) and National Science Foundation (NSF). The government has certain rights in the invention.

### COLOR DRAWINGS

The patent or application file contains at least one drawing executed in color. Copies of this patent or patent application publication with color drawing(s) will be provided by the Office upon request and payment of the necessary fee.

### BACKGROUND

Medical conditions such as Chronic Obstructive Pulmonary Disease (COPD) is an increasingly prevalent respiratory disorder, which represents a severe impediment for the quality of life. It is the third or fourth major cause of death worldwide. Medical practice presents COPD as an inflammatory lung condition including of a slow, progressive obstruction of airways that reduces pulmonary capacity. Medical science has not entirely clarified what triggers COPD. Nonetheless, scientists indicate the complex interactions between the environmental factors—such as pollution exposure or smoking—and the genetics as likely causes. COPD is not reversible, but early diagnosis creates incentives for achieving a better disease evolution, and an improved patient condition through personalized treatments.

### SUMMARY

In some arrangements, systems, methods, non-transitory computer-readable media, and apparatuses include generating physiological signals datasets, analyzing physiological signals in the physiological signals datasets, extracting fractional dynamics signatures specific to Chronic Obstructive Pulmonary Disease (COPD) medical records, and identifying, using a deep neural network (DNN), a COPD stage.

In some arrangements, the physiological signals datasets comprises a WestRo COPD dataset and a WestRo Porti COPD dataset. In some arrangements, the physiological signals is recorded using at least one Internet of Medical Things (IoMT) device. In some arrangements, the DNN is trained to identify one of a plurality of COPD stages using fractal dynamic network signatures and expert analysis. In some arrangements, a fractional dynamics deep learning model (FDDLM) is constructed. The FDDLM is trained using a training set to recognize COPD level based on signal signatures. The FDDLM is tested using a test set to predict one of a plurality of COPD stages, wherein the DNN comprises the FDDLM.

In some arrangements, fractional-order dynamical modeling is performed. Distinguishing signatures is extracted from the physiological signals across patients with all COPD stages. The COPD stage is identified using at least one of thorax breathing effort, respiratory rate, or oxygen saturation levels. The input of the DNN comprises features extracted from a fractional-order dynamic model.

In some arrangements, a system includes a memory and a processor configured to generate physiological signals datasets, analyze physiological signals in the physiological signals datasets, extract fractional dynamics signatures specific to Chronic Obstructive Pulmonary Disease (COPD) medical records, and identify, using a deep neural network (DNN), a COPD stage.

In some arrangements, a non-transitory processor-readable medium comprising processor-readable instructions, such that, when executed by a processor, causes the processor to generate physiological signals datasets, analyze physiological signals in the physiological signals datasets, extract fractional dynamics signatures specific to Chronic Obstructive Pulmonary Disease (COPD) medical records, and identify, using a deep neural network (DNN), a COPD stage.

### BRIEF DESCRIPTION OF THE DRAWINGS

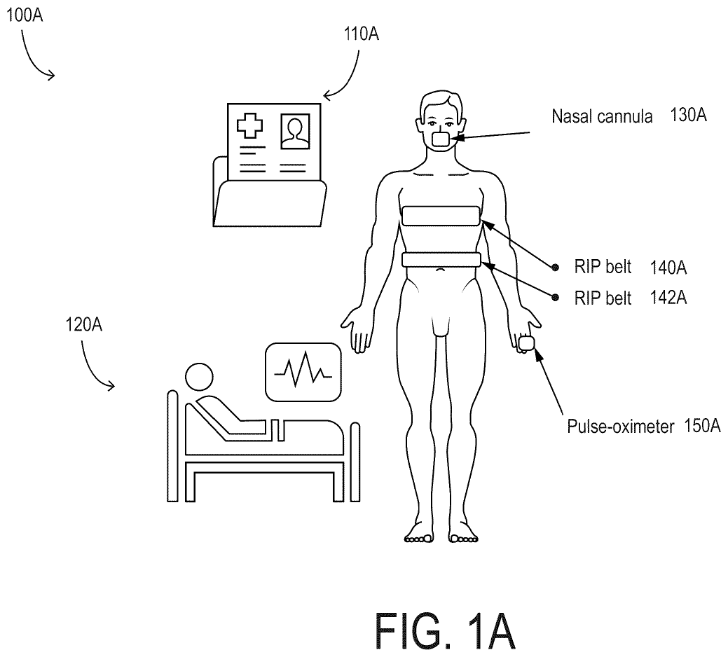

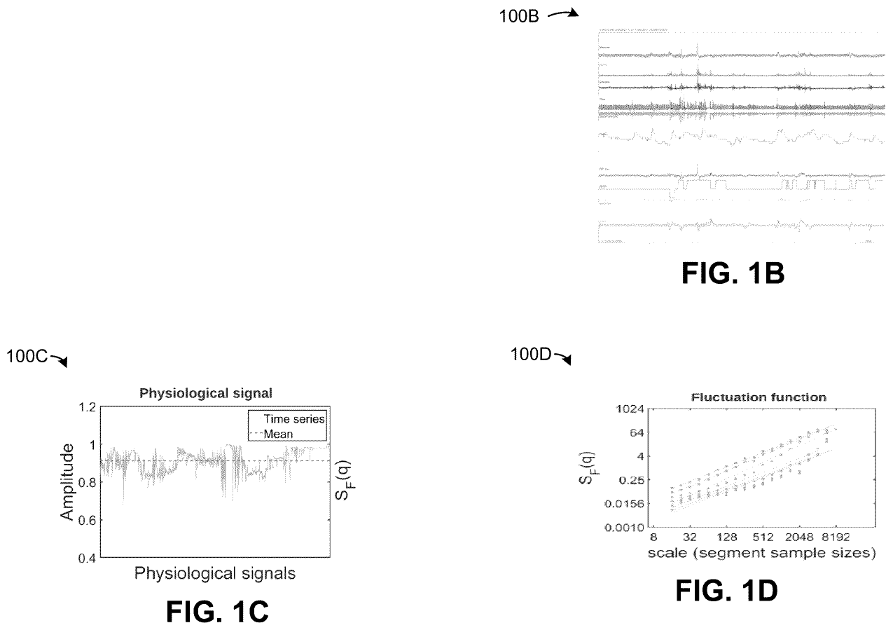

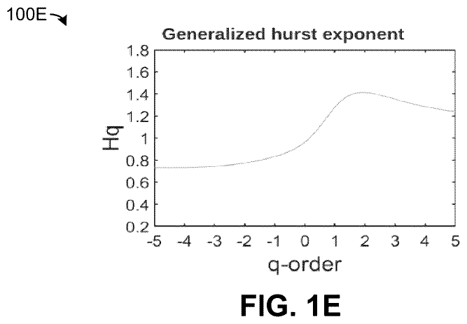

FIGS. 1A, 1B, 1C, 1D, 1E, and 1F illustrate an example method for COPD stage prediction.

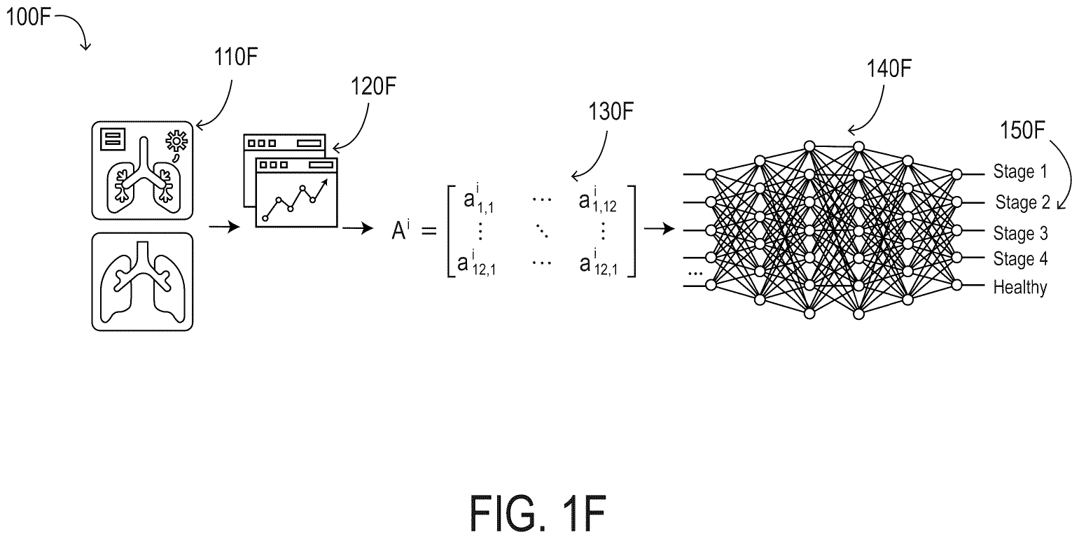

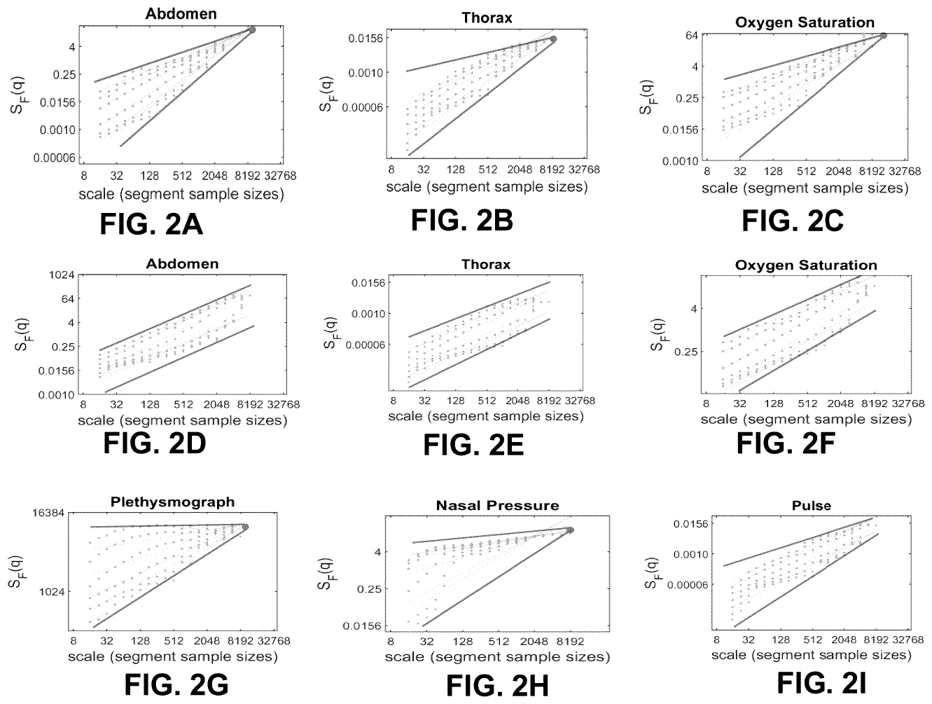

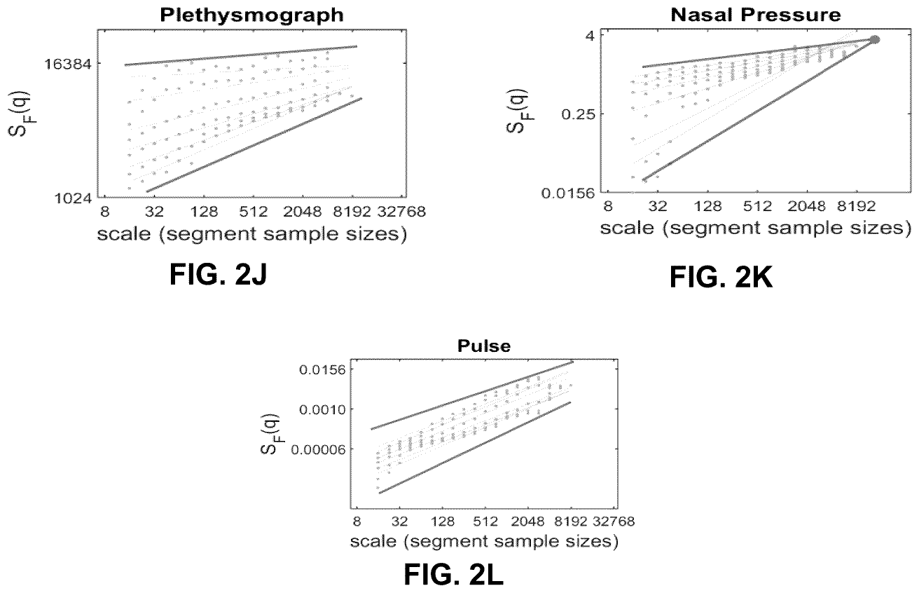

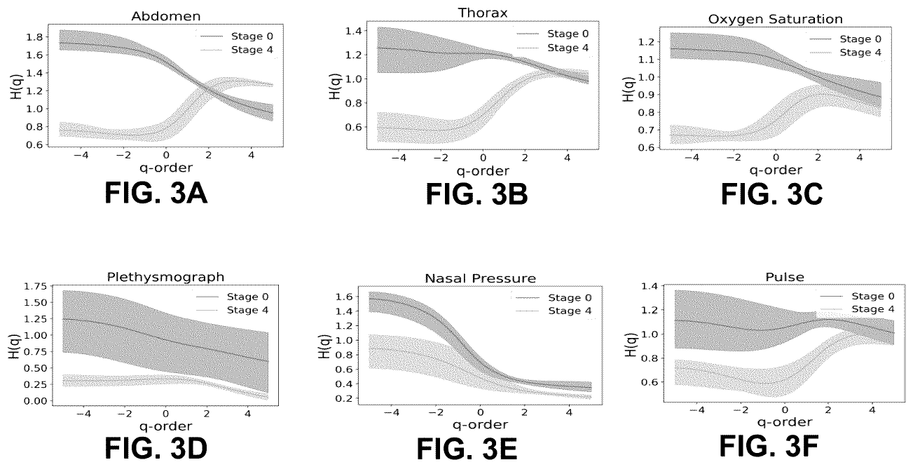

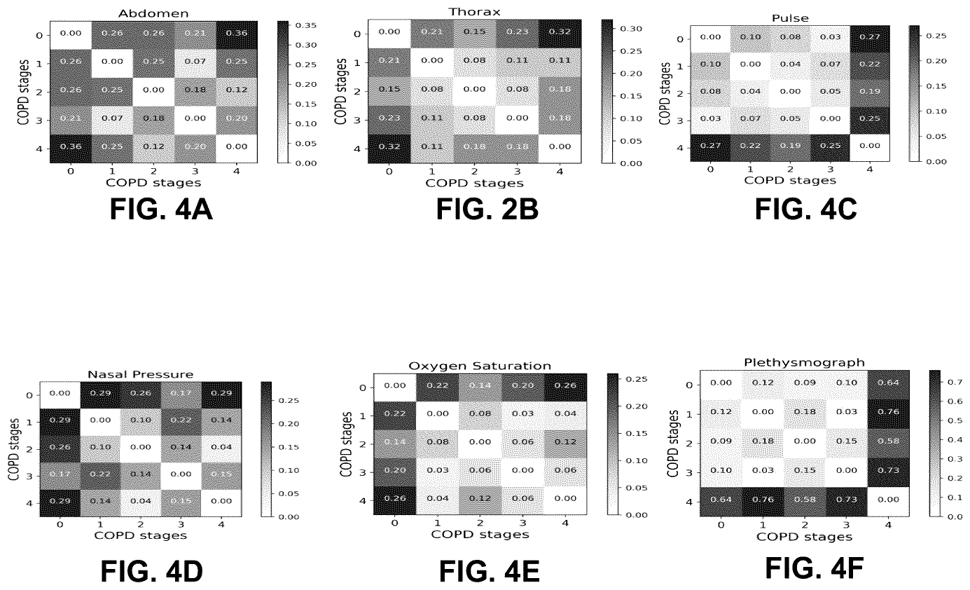

FIGS. 2A, 2B, 2C, 2D, 2E, 2F, 2G, 2H, 2I, 2J, 2K, and 2L illustrate various scaling functions.

FIGS. 3A, 3B, 3C, 3D, 3E, and 3F illustrate H(q) comparisons between physiological signals.

FIGS. 4A, 4B, 4C, 4D, 4E, and 4F illustrate, for each physiological signal, signals' H(q) function extracted from patients in various COPD stages.

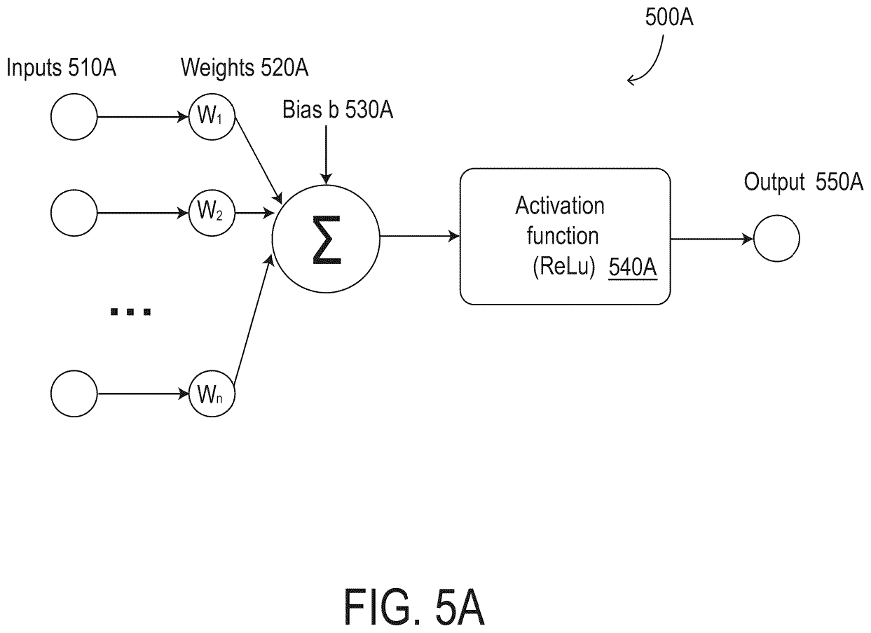

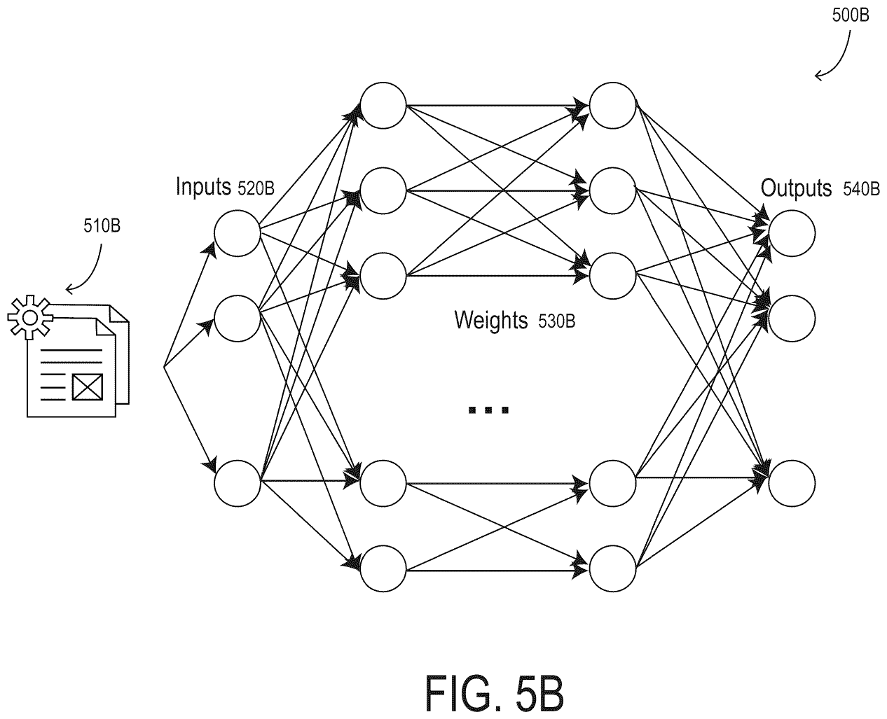

FIGS. 5A and 5B illustrate an example model for determining COPD stages.

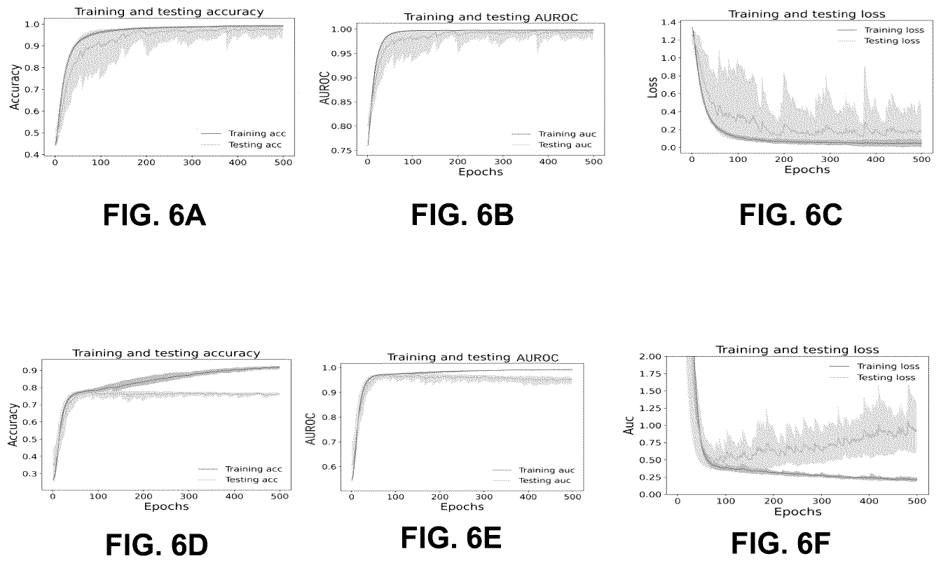

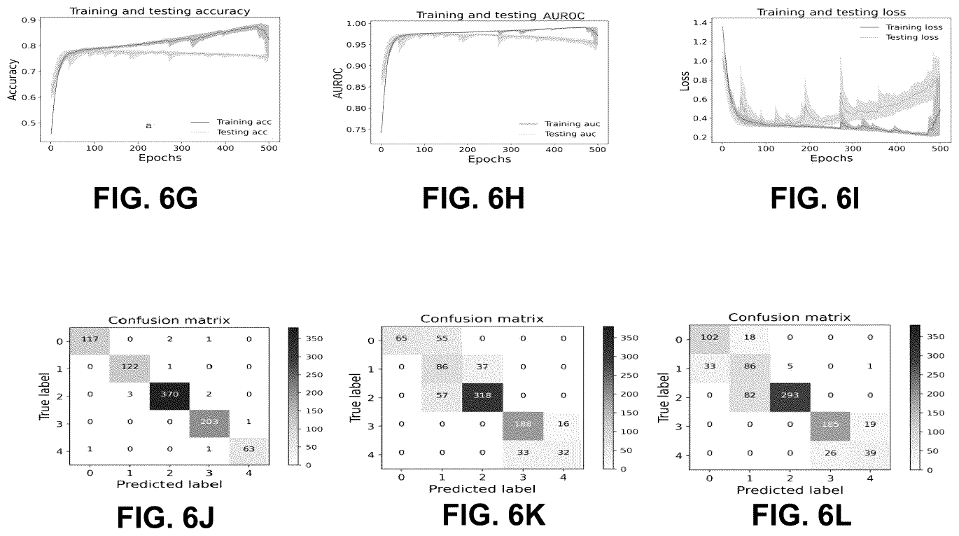

FIGS. 6A, 6B, 6C, 6D, 6E, 6F, 6G, 6H, 6I, 6J, 6K, 6L show training and testing result comparisons of different deep learning models for the k-fold cross-validation.

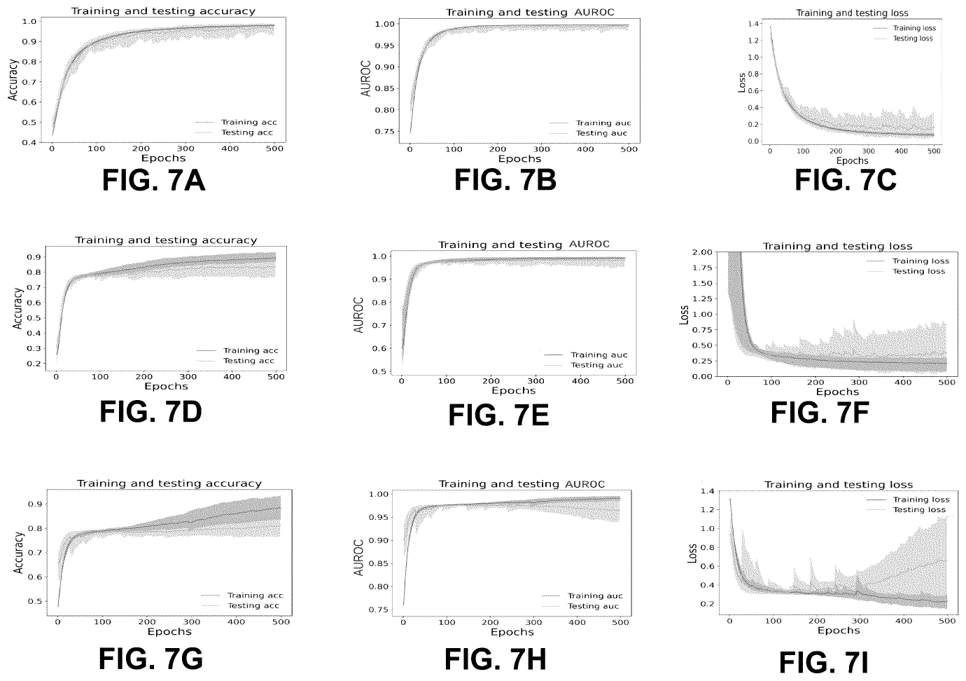

FIGS. 7A, 7B, 7C, 7D, 7E, 7F, 7G, 7H, 7I show training and testing result comparisons of different deep learning models for the hold-out validation.

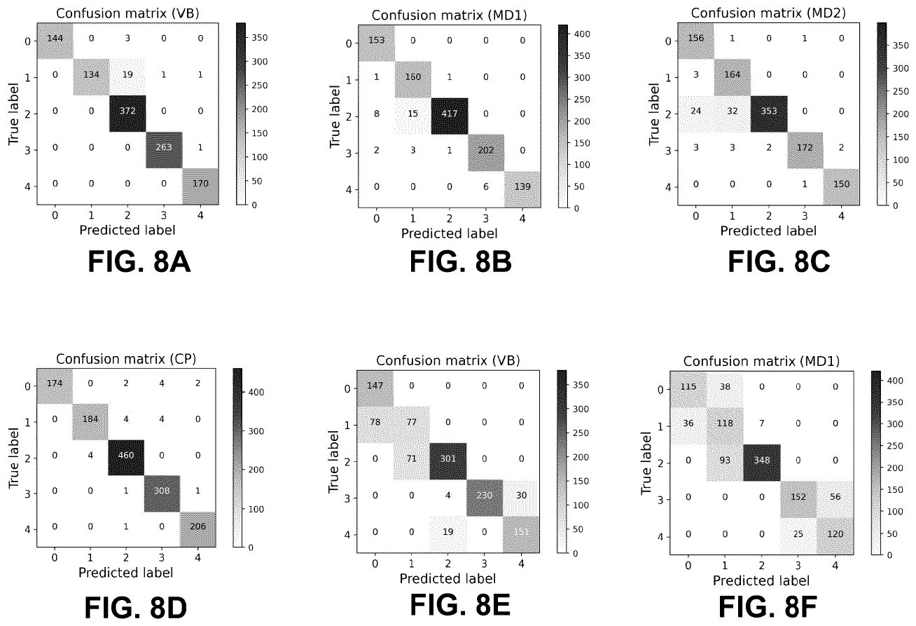

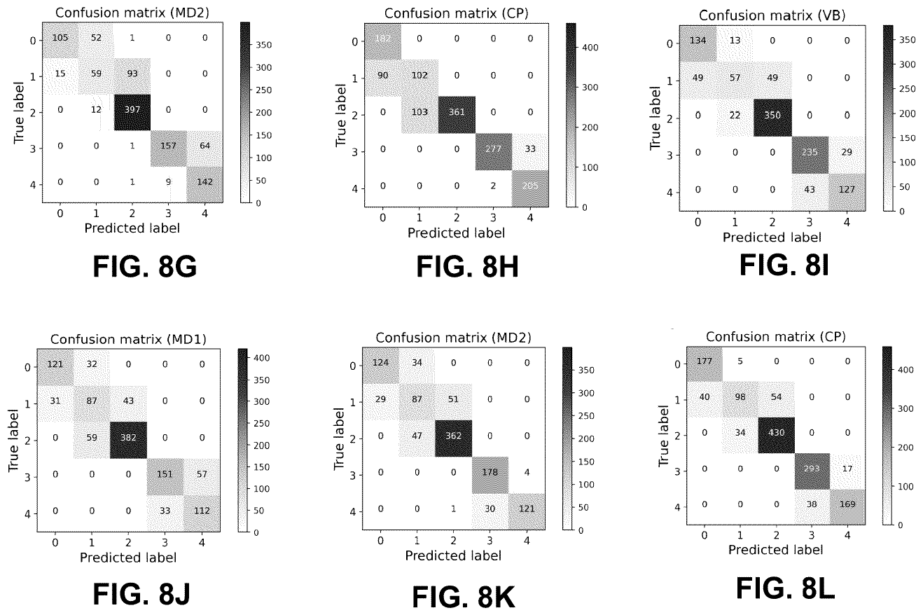

FIGS. 8A, 8B, 8C, 8D, 8E, 8F, 8G, 8H, 8I, 8J, 8K, 8L illustrate the comparison of confusion matrices resulted from different deep learning models.

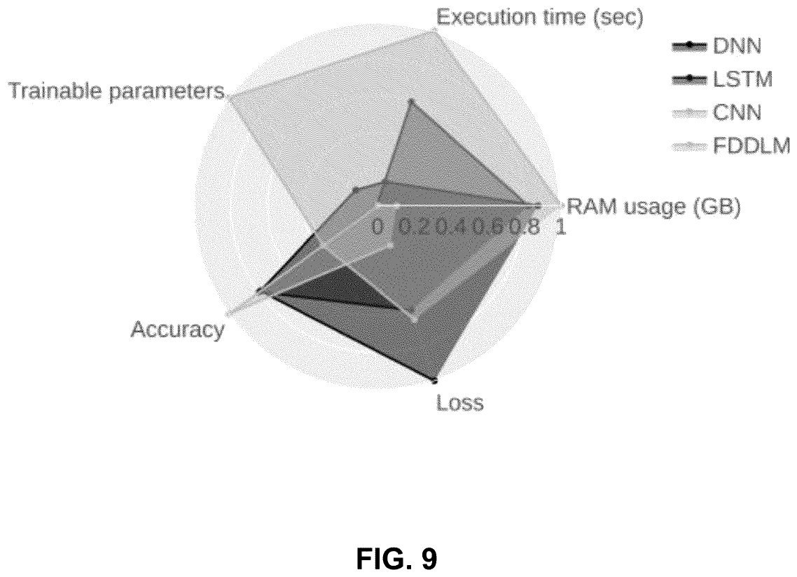

FIG. 9 is a radar plot for measuring the complexity and prediction performance for the WestRo COPD dataset across different deep learning models under k-fold validation (k=5).

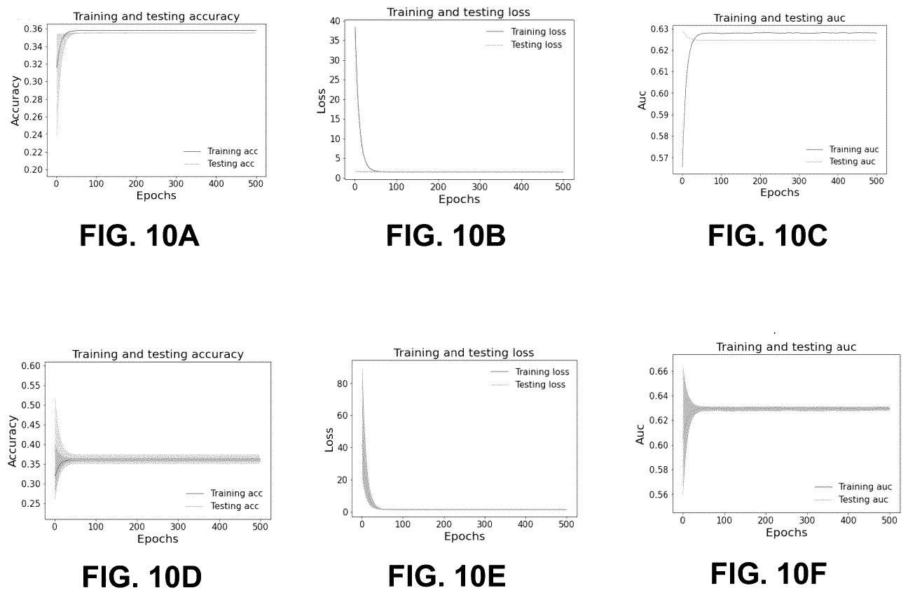

FIGS. 10A, 10B, 10C, 10D, 10E, 10F illustrate training and testing results in terms of accuracy, loss, and AUC of the convolutional neural network model for the k-fold cross-validation and hold out validation.

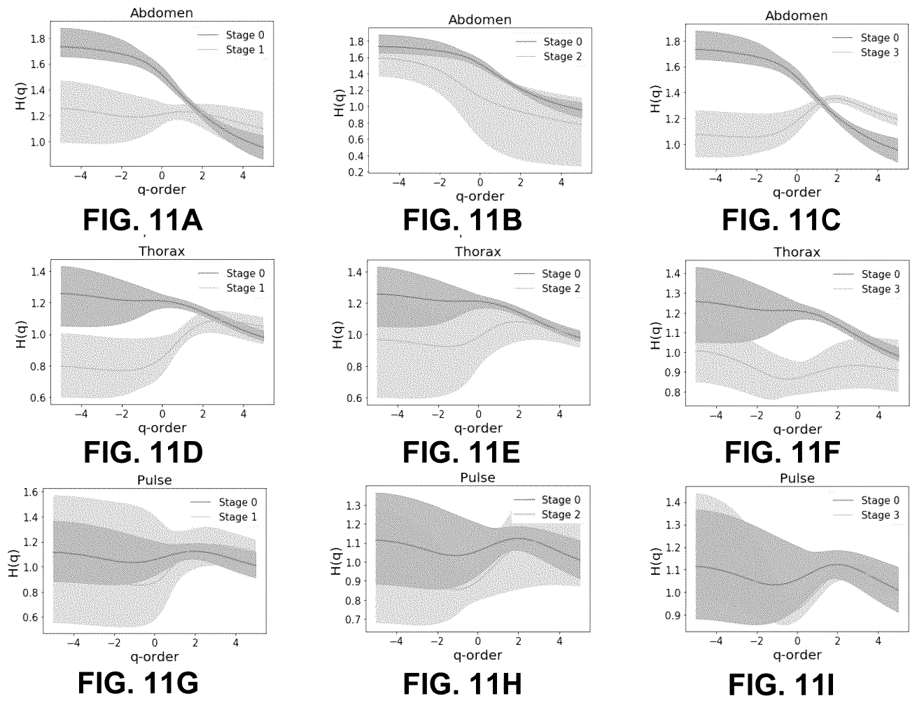

FIGS. 11A, 11B, 11C, 11D, 11E, 11F, 11G, 11H, and 11I present the H(q) confidence intervals for Abdomen, Thorax, and Pulse signals, for different q values, across different COPD stages (i.e., 0, 1, 2, 3) with 95% confidence intervals.

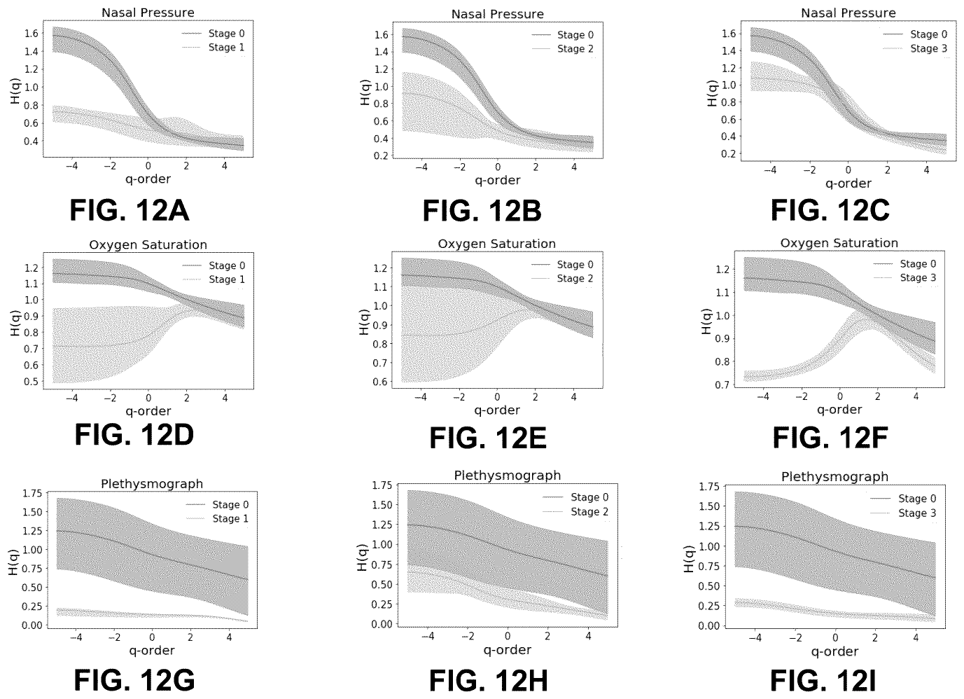

FIGS. 12A, 12B, 12C, 12D, 12E, 12F, 12G, 12H, 12I presents the H(q) confidence intervals for Nasal Pressure, Oxygen Saturation, and Plethysmograph signals, for different q values, across different stages (0, 1, 2, 3) with 95% confidence intervals.

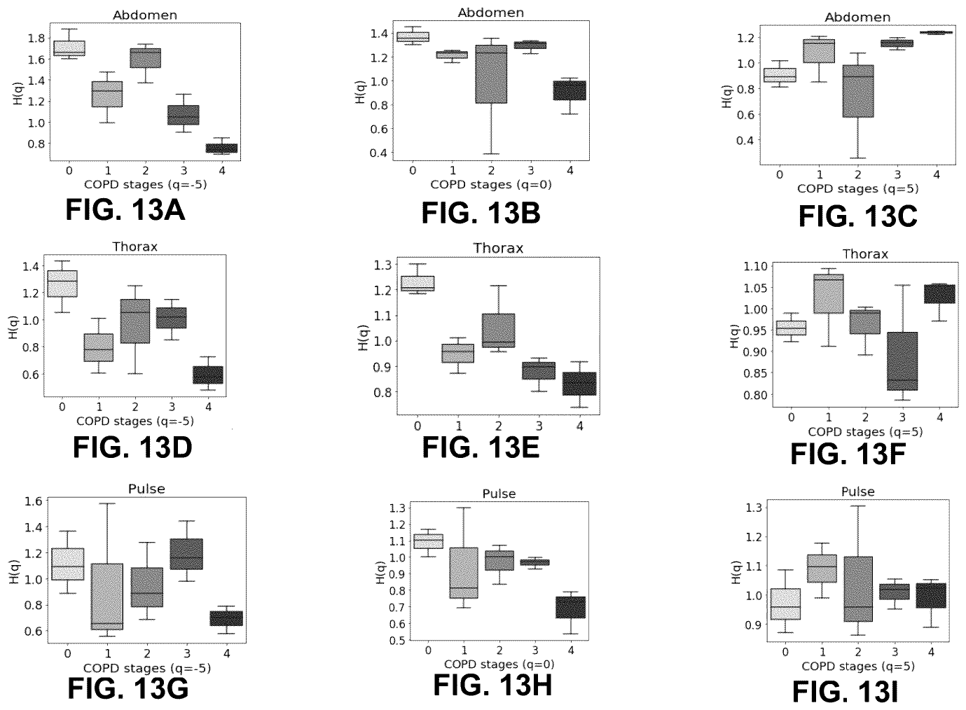

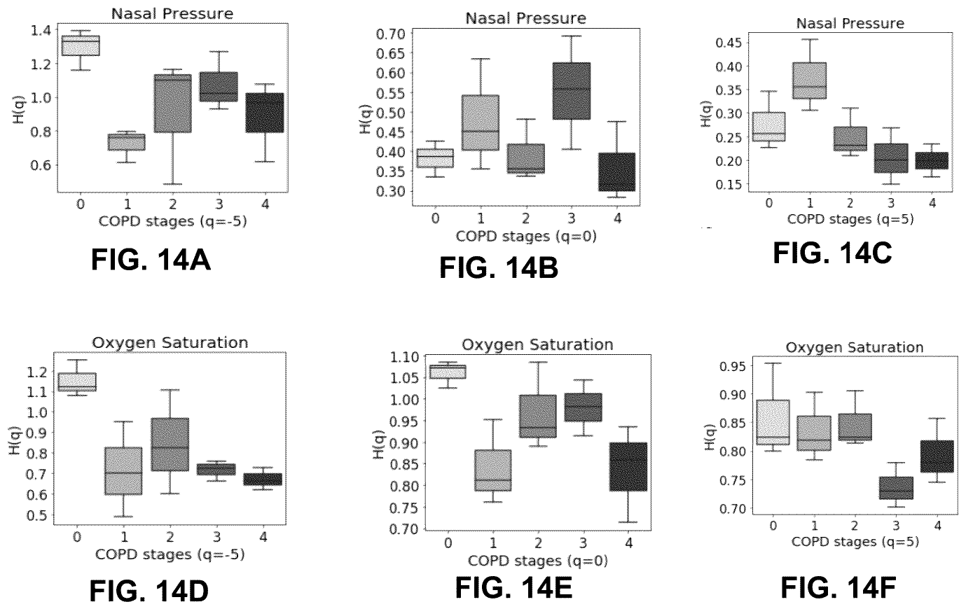

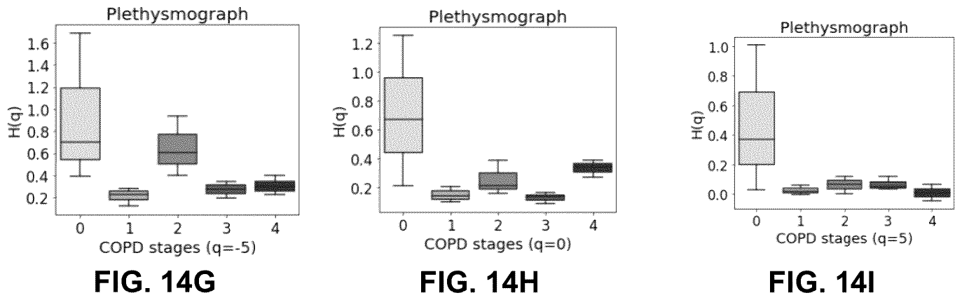

FIGS. 13A, 13B, 13C, 13D, 13E, 13F, 13G, 13H, 13I and FIGS. 14A, 14B, 14C, 14D, 14E, 14F, 14G, 14H, 14I present the comparison of terms of H(q) intervals for COPD patients for different q values (q∈{−5, 0, 5}.

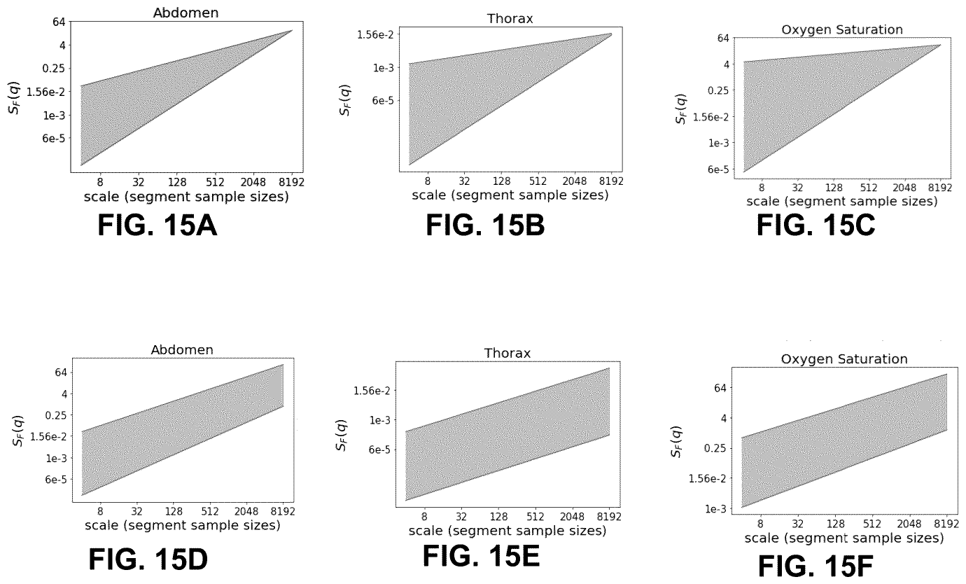

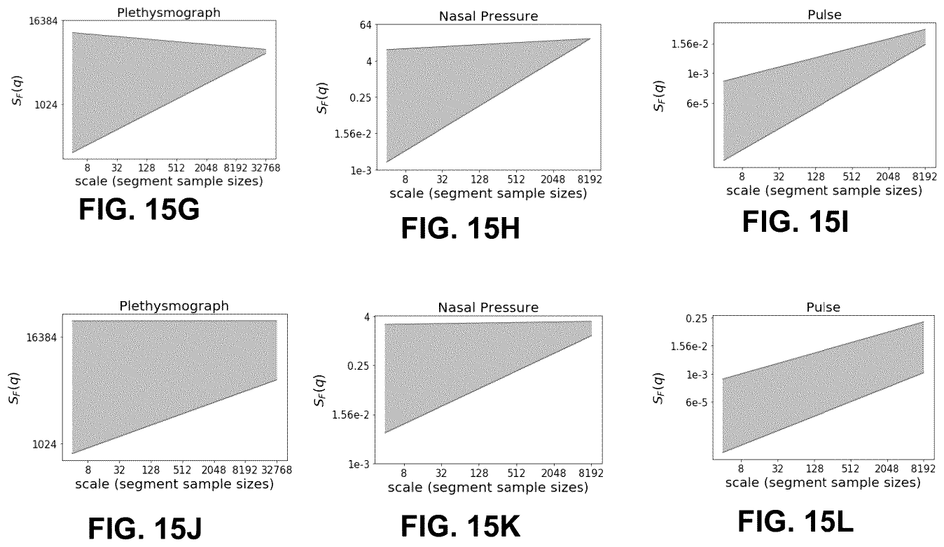

FIGS. 15A, 15B, 15C, 15D, 15E, 15F, 15G, 15H, 15I, 15J, 15K, 15L present the comparison of terms of H(q) intervals for COPD patients for different q values (q∈{−5, 0, 5}.

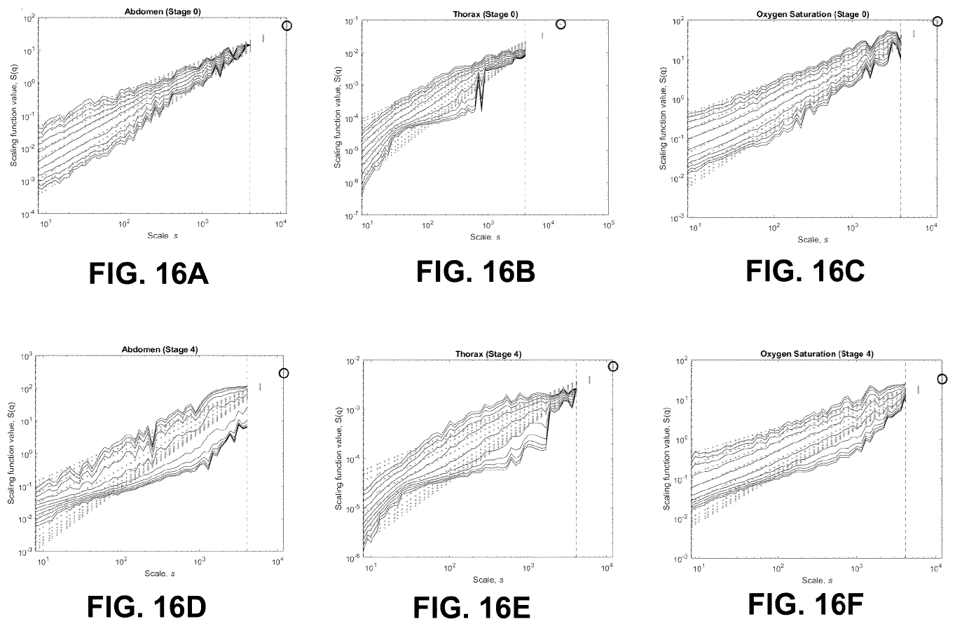

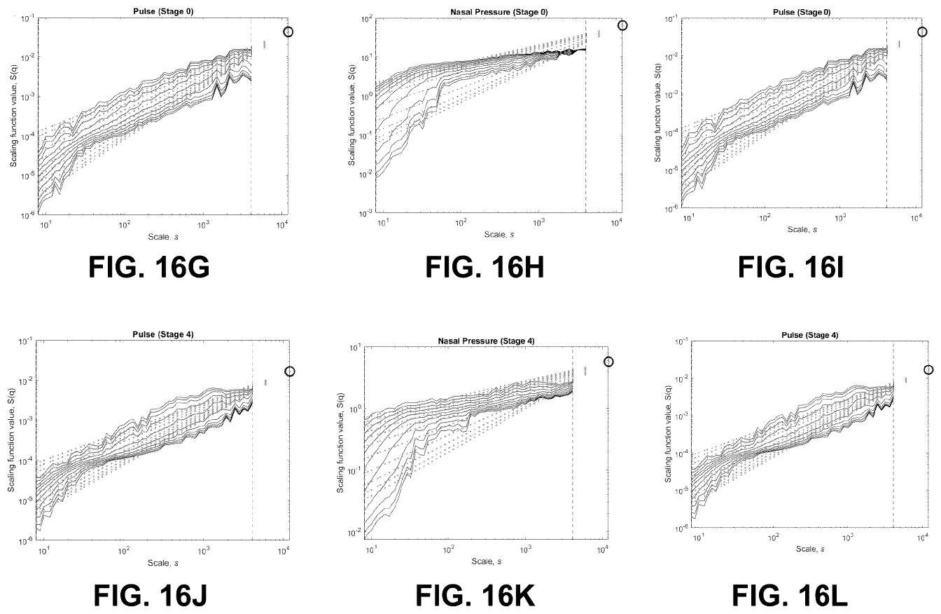

FIGS. 16A, 16B, 16C, 16D, 16E, 16F, 16G, 16H, 16I, 16J, 16K, 16L illustrate the geometry of fluctuation profiles for the COPD-relevant physiological signals recorded from a normal abdomen and a stage 4 COPD abdomen.

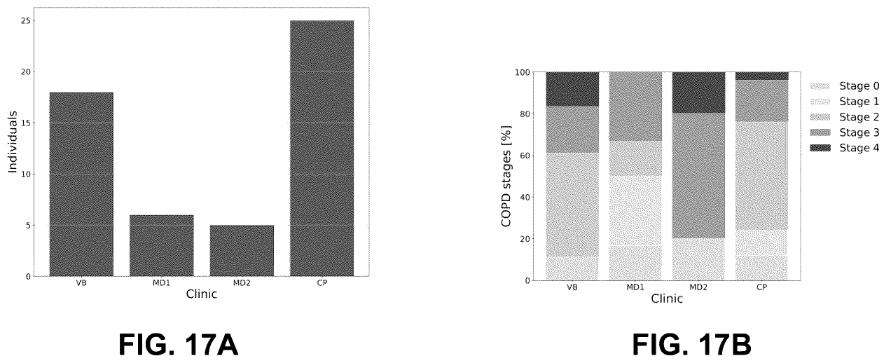

FIG. 17A illustrates data distribution across the medical institutions. FIG. 17B illustrates percentage of COPD stages for each medical institution.

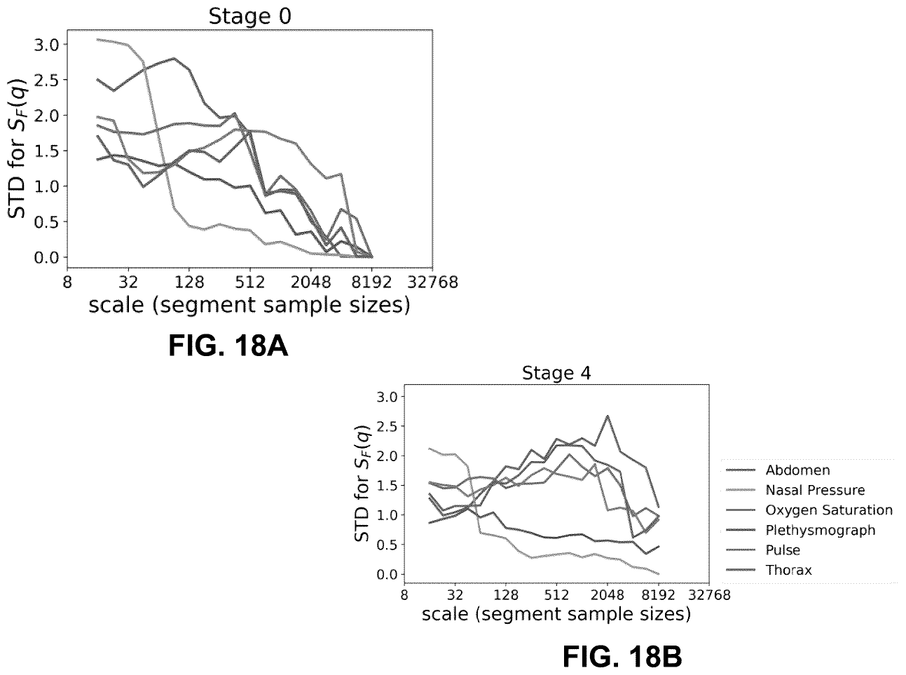

FIGS. 18A and 18B illustrate scale (s) vs. standard deviation (STD) of S_{F}(q) between stage 0 and stage 4 participants.

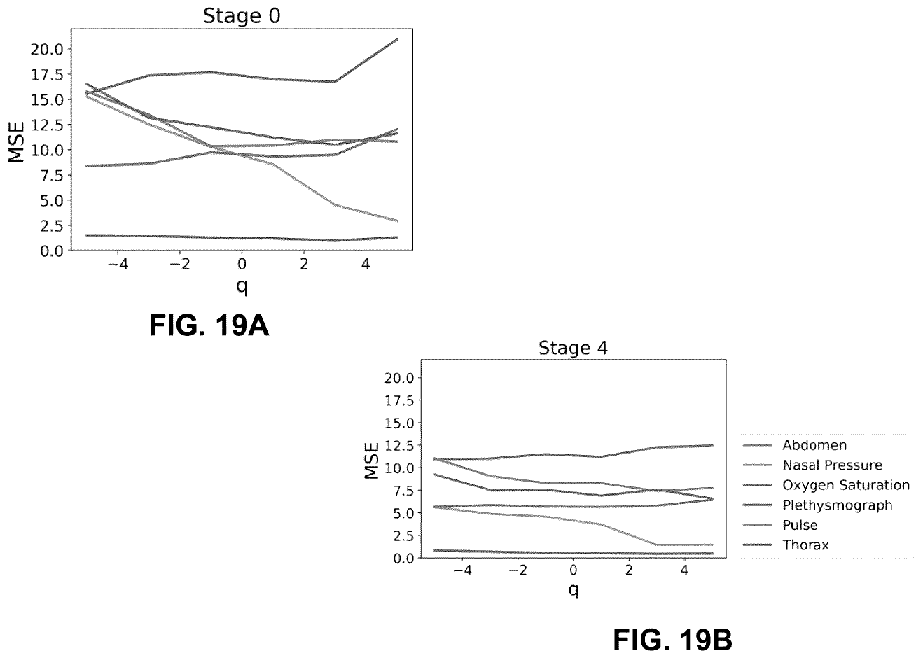

FIGS. 19A and 19B show the MSE for stage 0 and 4 participants, respectively.

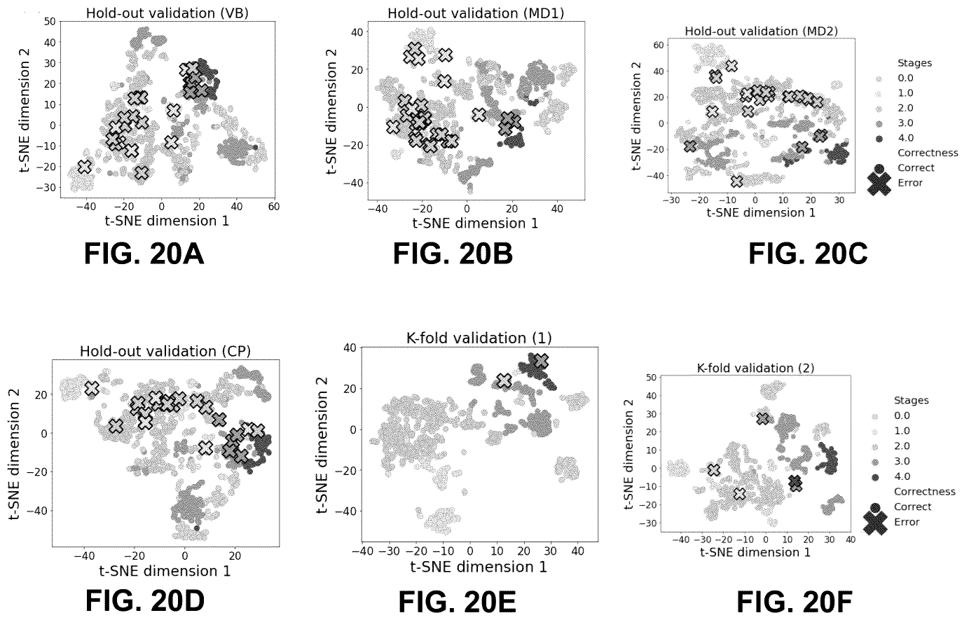

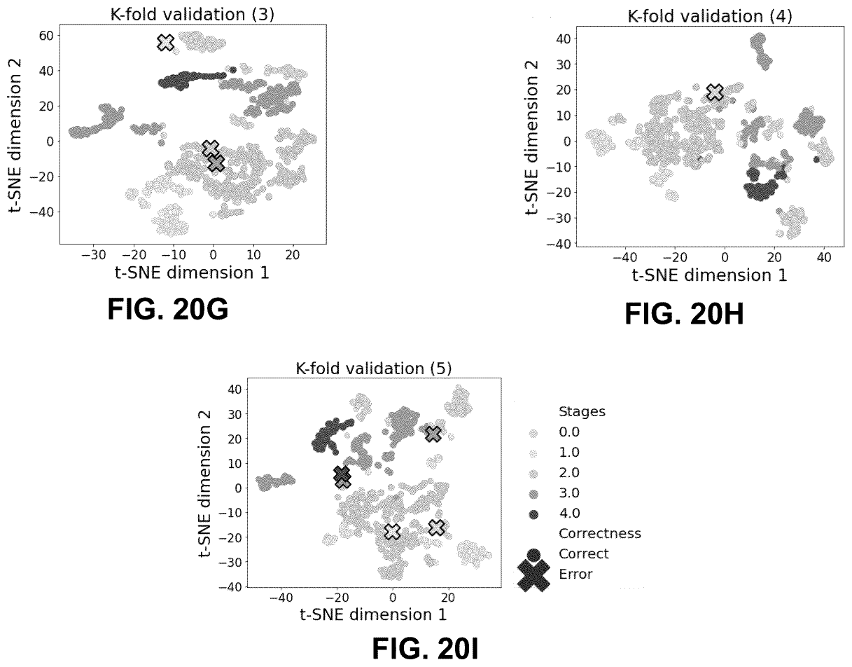

FIG. 20A, 20B, 20C, 20D, 20E, 20F, 20G, 20H, 20I illustrate representation of the learning process about hold-out and k-fold cross-validation in the first hidden layer.

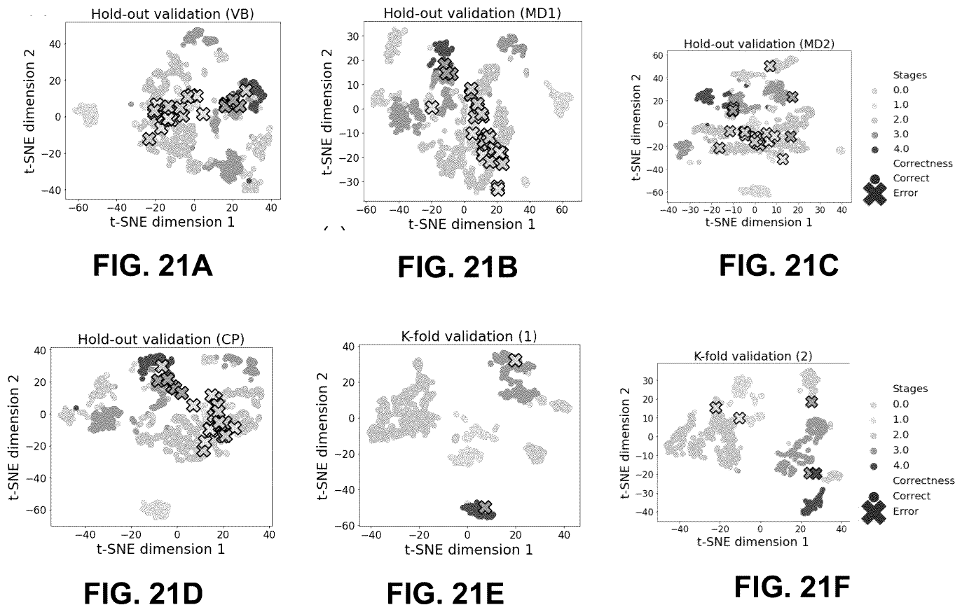

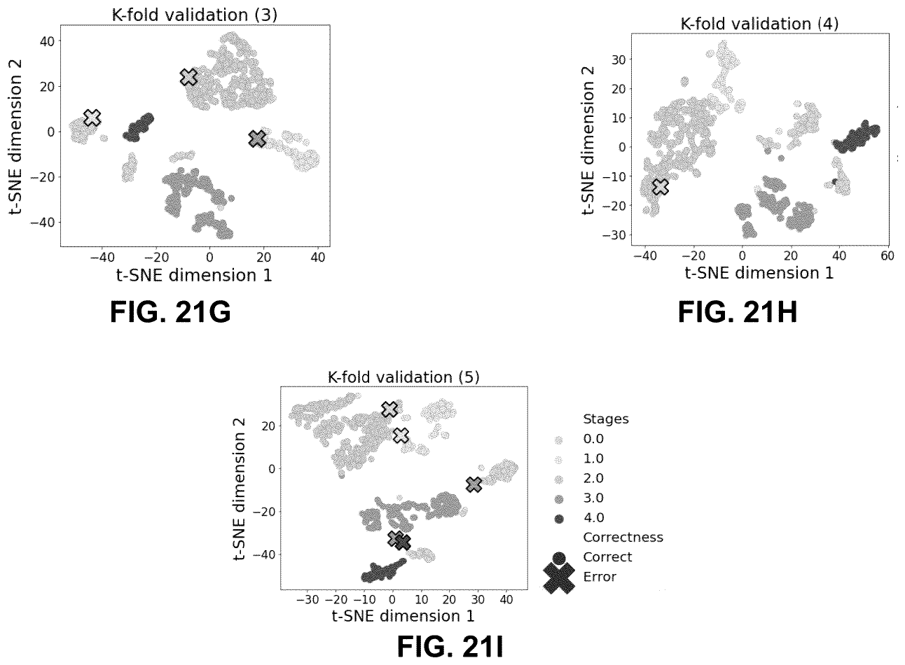

FIGS. 21A, 21B, 21C, 21D, 21E, 21F, 21G, 21H, 21I illustrate representation of the learning process about hold-out and k-fold cross-validation in the last hidden layer.

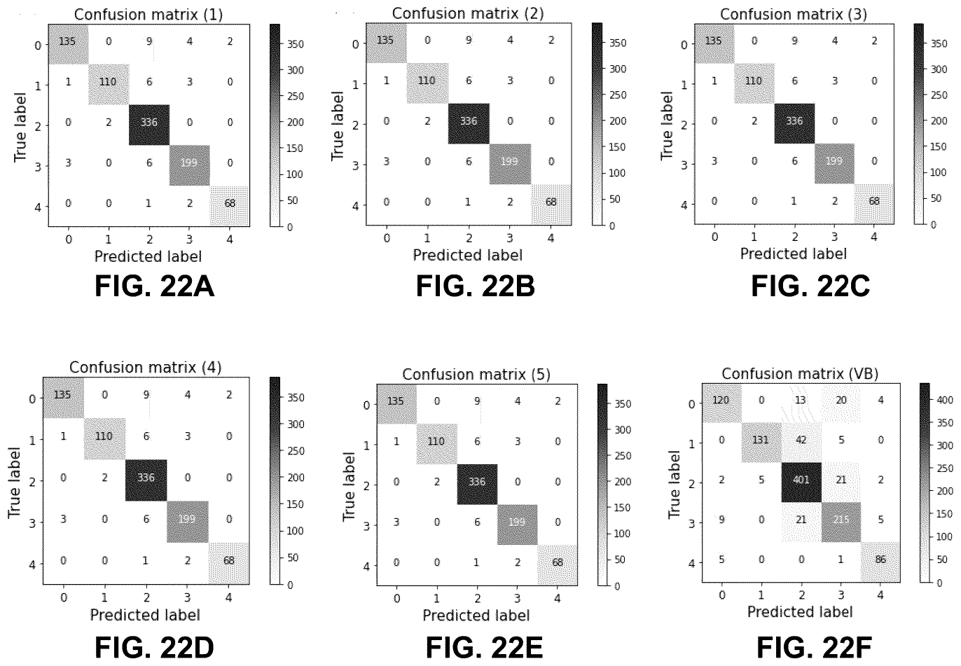

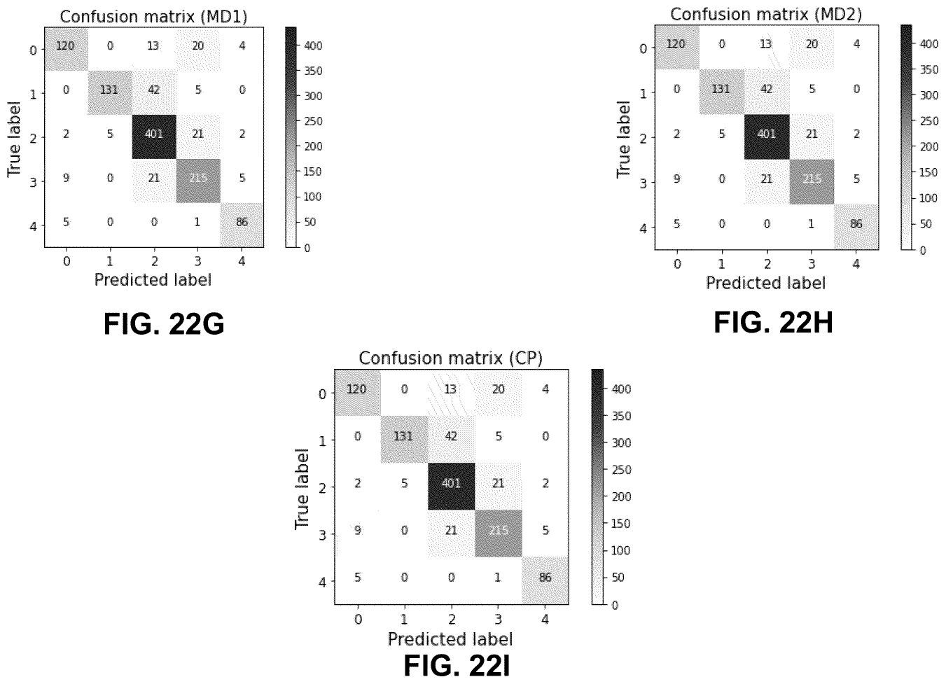

FIGS. 22A, 22B, 22C, 22D, 22E, 22F, 22G, 22H, 22I illustrate the confusion matrices resulted from the logistic regression model.

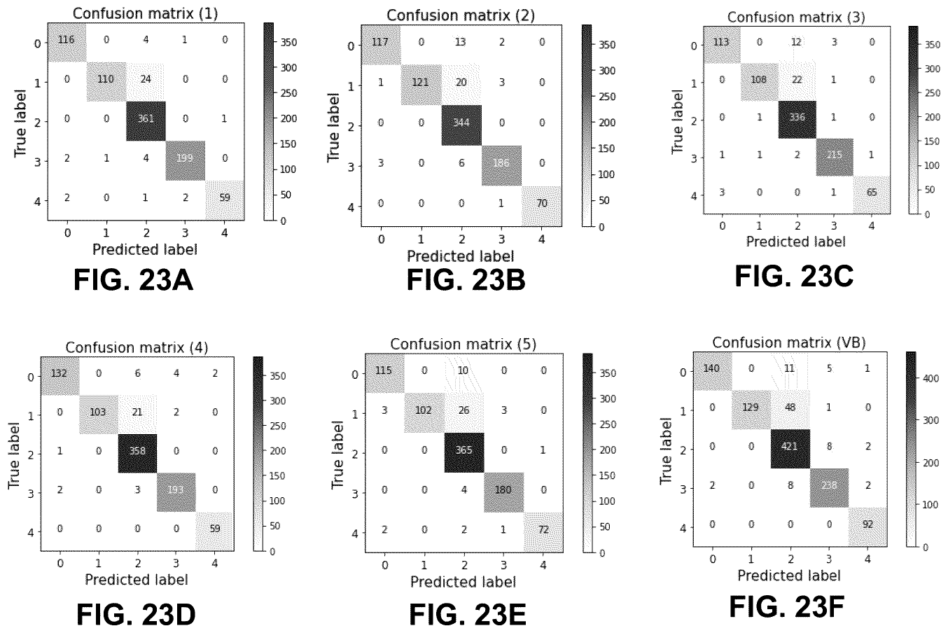

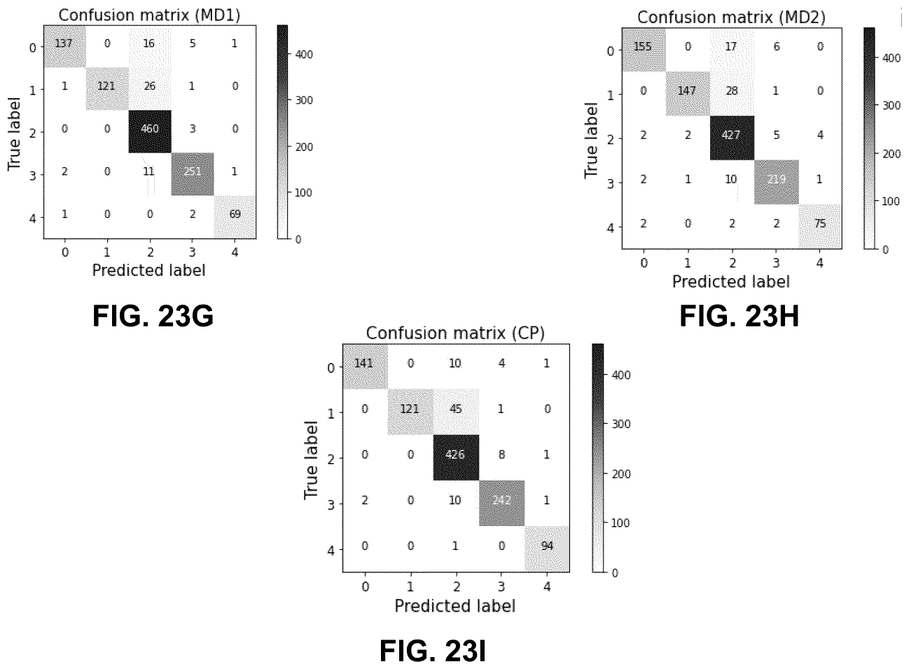

FIGS. 23A, 23B, 23C, 23D, 23E, 23F, 23G, 23H, 23I illustrate the confusion matrices resulted for the SVM. For the k-fold cross validation (k=5).

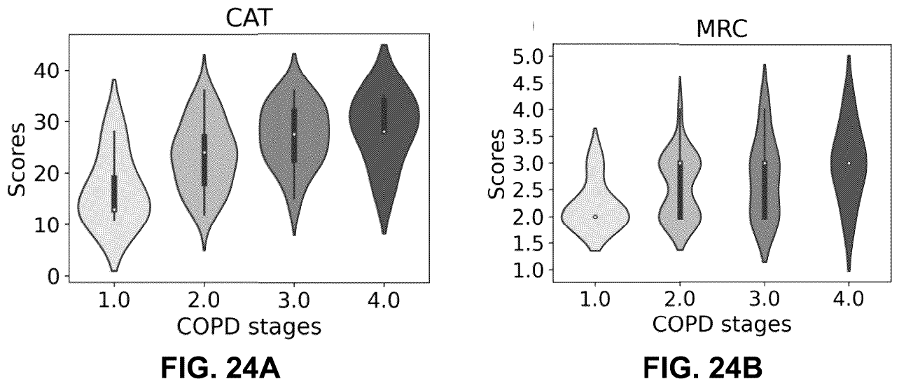

FIG. 24A illustrates CAT scores for COPD patients across different COPD stages. FIG. 24B illustrates MRC scores for COPD patients across different COPD stages.

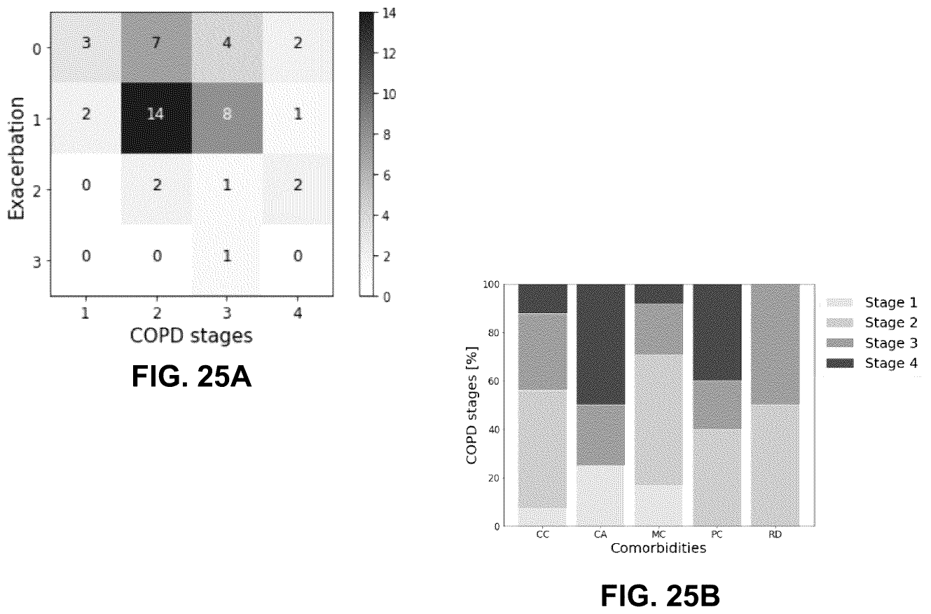

FIG. 25A illustrates exacerbations across patients under different COPD stages. FIG. 25B illustrates a percentage of COPD stages for each comorbidity.

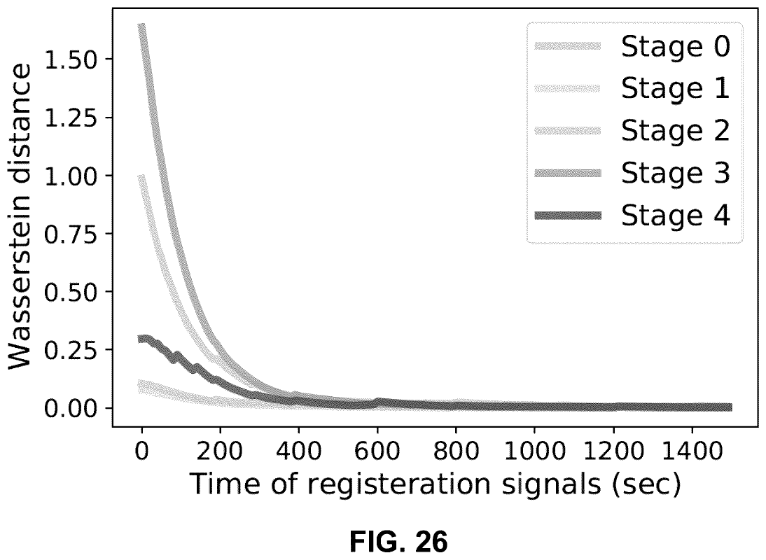

FIG. 26 displays the convergence of matrix A across different stages of patients.

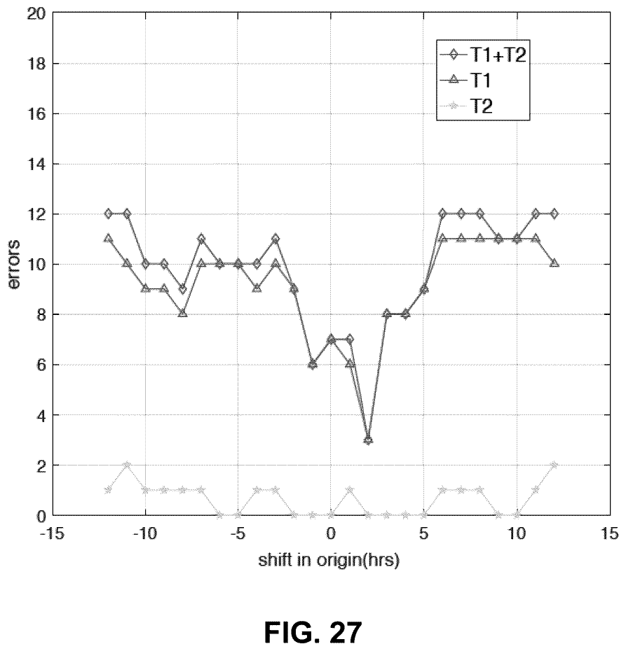

FIG. 27 shows the plot of classification performance in terms of Type-I and Type-II errors when the inoculation point is shifted in positive and negative increments.

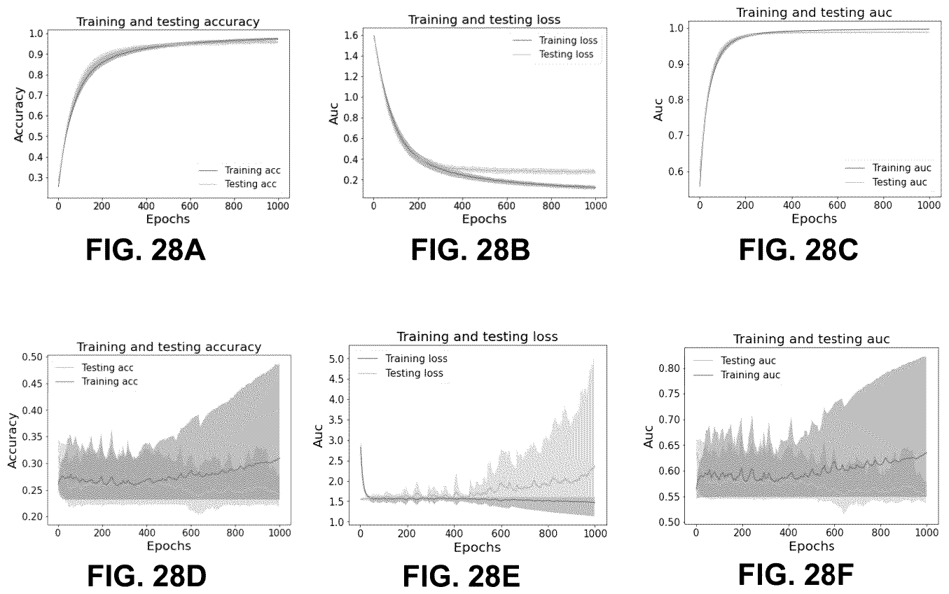

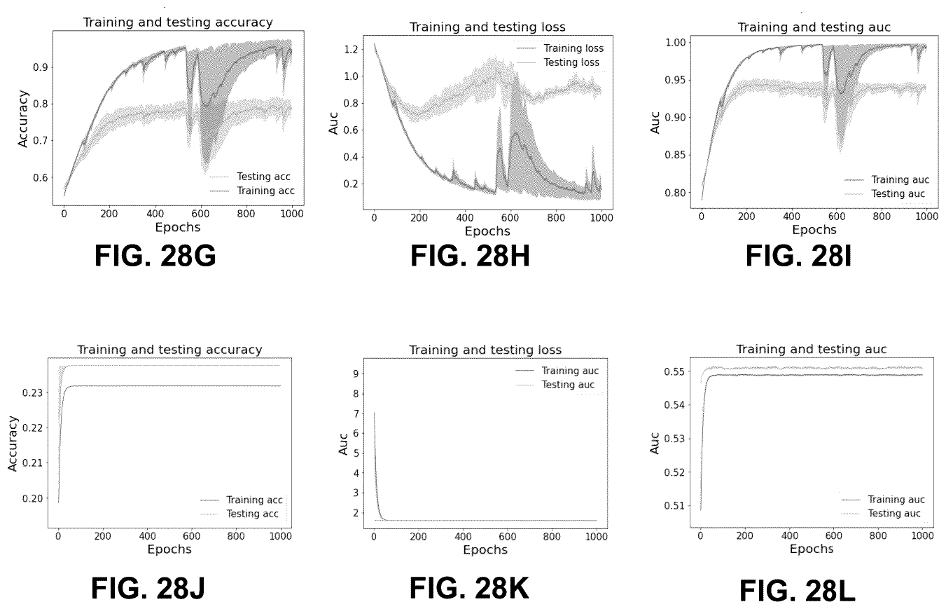

FIGS. 28A, 28B, 28C, 28D, 28E, and 28F illustrate training and testing result comparisons of different deep learning models (WestRo Porti dataset) for the k-fold cross-validation (k=5).

FIGS. 28G, 28H, 28I respectively, present the training and testing accuracy, loss, and AUC results curves for the LSTM model trained with physiological signals (raw data).

FIGS. 28J, 28K, and 28L illustrate training and testing result comparisons of different deep learning models (WestRo Porti dataset) for the k-fold cross-validation (k=5).

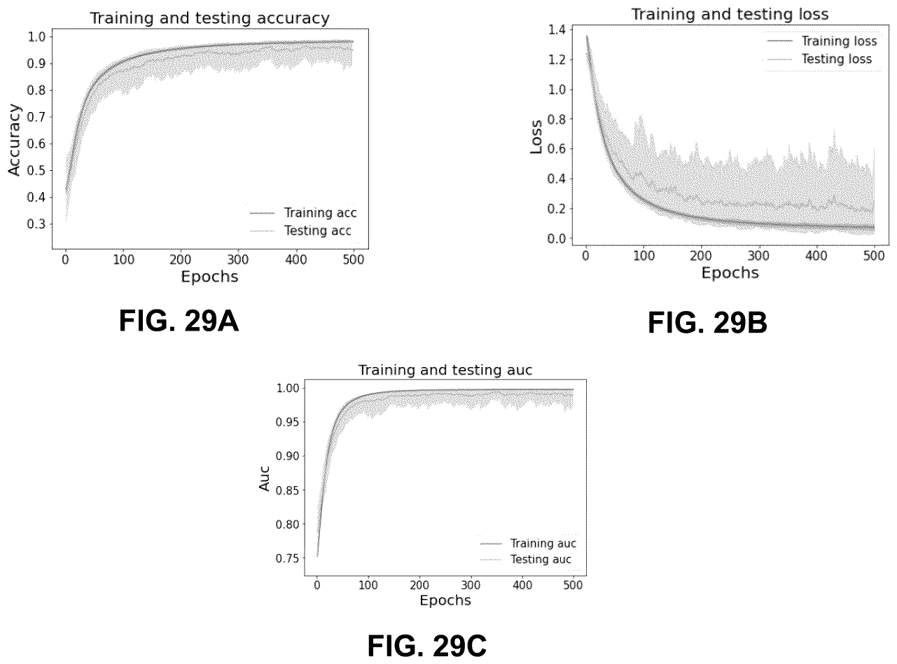

FIGS. 29A, 29B, and 29C presents our model's performance as accuracy, loss, and AUC curves between training and testing datasets (where k=5).

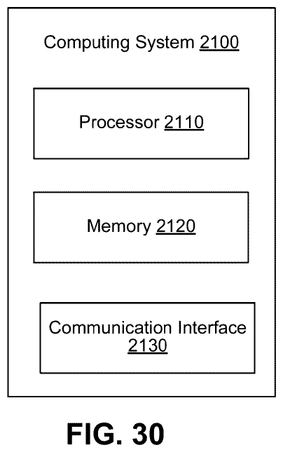

FIG. 30 is a block diagram illustrating an example computing system for implementing the fractional dynamics foster deep learning of COPD stage prediction.

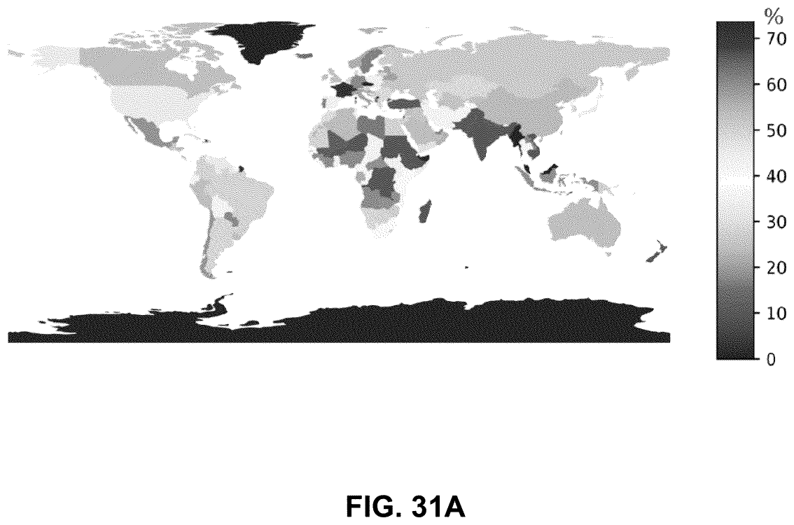

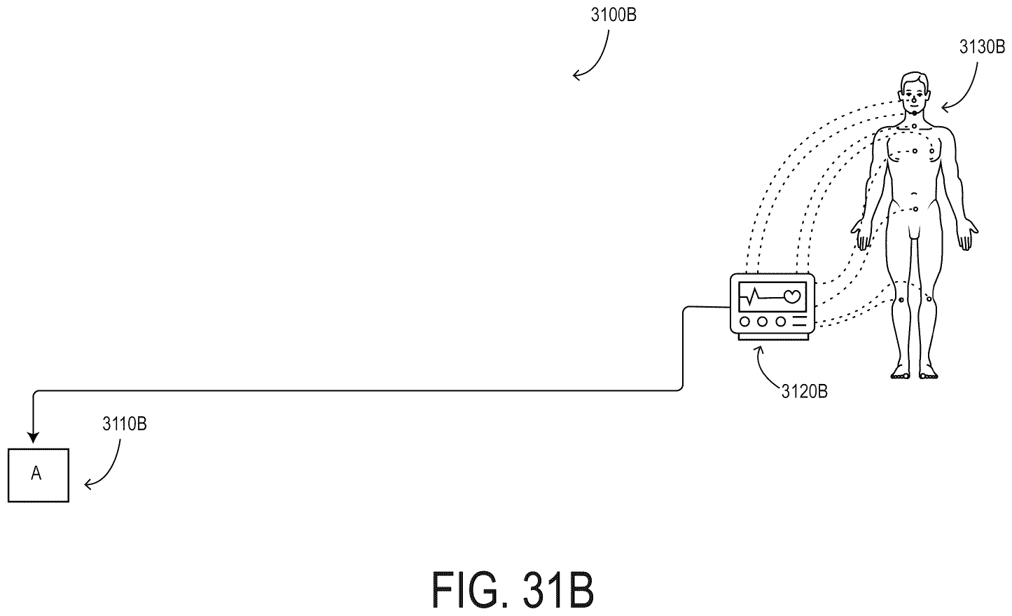

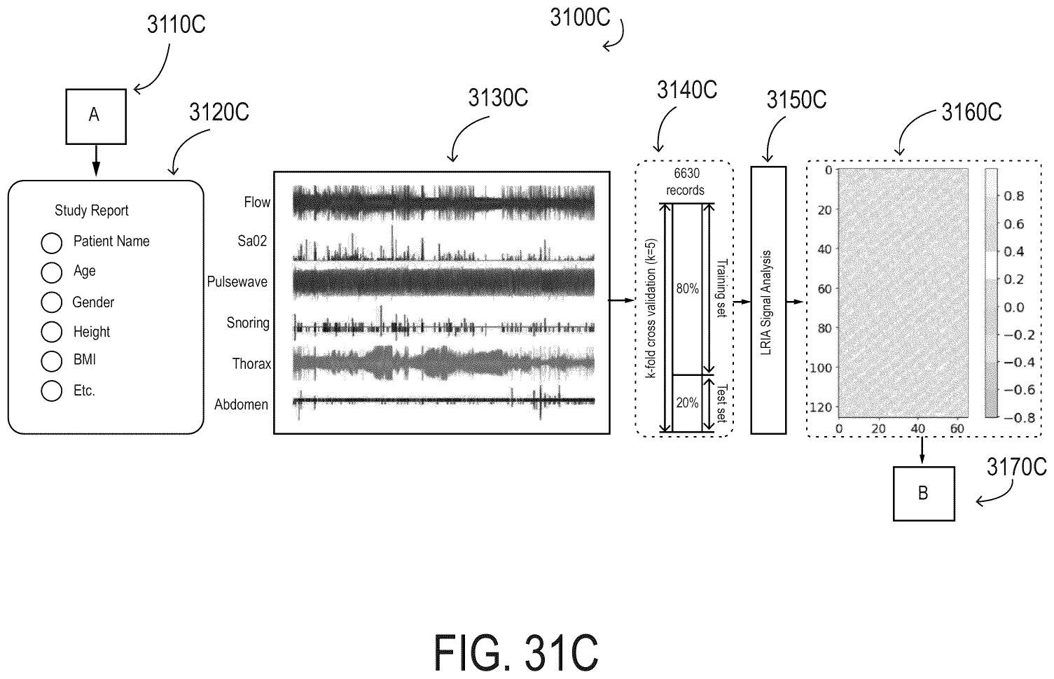

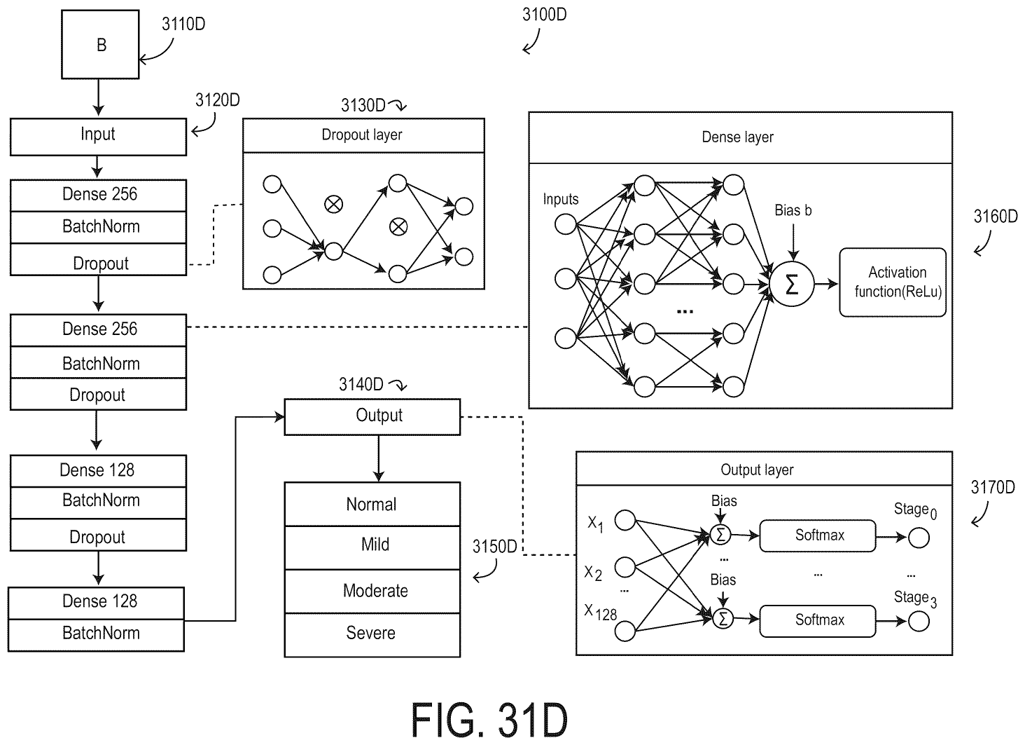

FIG. 31A is a map of the global prevalence of obstructive sleep apnea.

FIG. 31B is a diagram of physiological data recording devices, including an Alice 6 device by Philips Respironics and the Porti 8, used to collect overnight physiological signals from participants.

FIG. 31C is a diagram of six physiological data channels that are highly relevant to sleep apnea diagnosis with a sample time-series record of a patient and a LRIA analysis used to extract one or more coupling matrices from the physiological signals, which represent the input to the DNN model.

FIG. 31D is a diagram of the structure of a DNN model with detailed illustrations of various types of layers of the DNN model, which DNN model outputs the predicted stage from normal to severe.

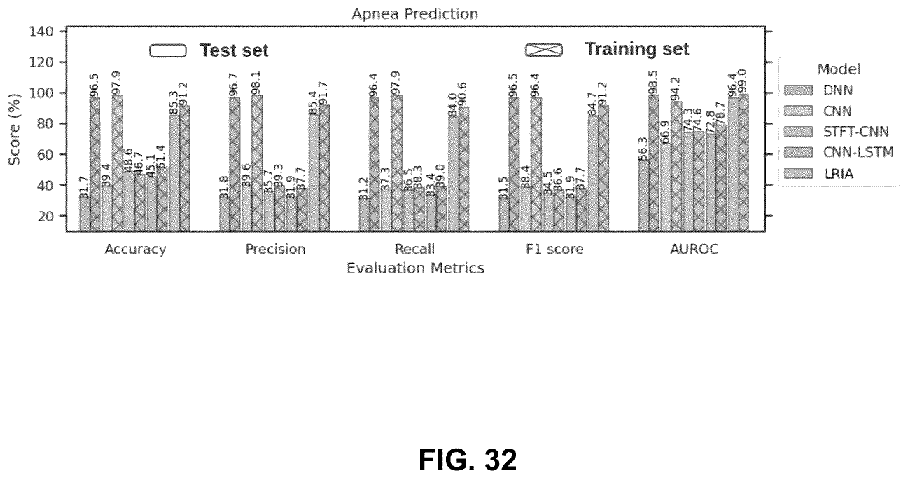

FIG. 32 is an illustration of the performance comparison between a LRIA analysis model and other different deep learning models.

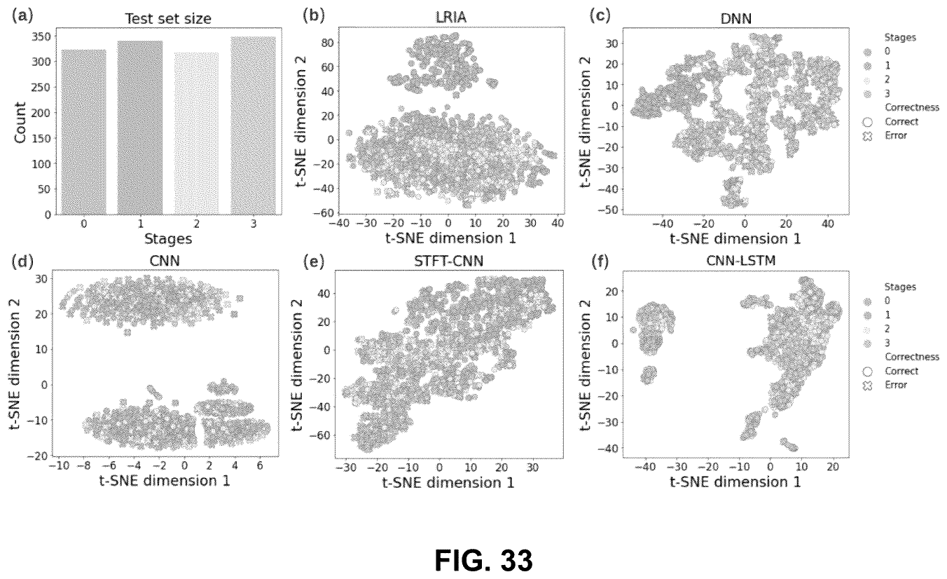

FIG. 33 is a representation of the learning process and test dataset distribution about different models in the last hidden layers.

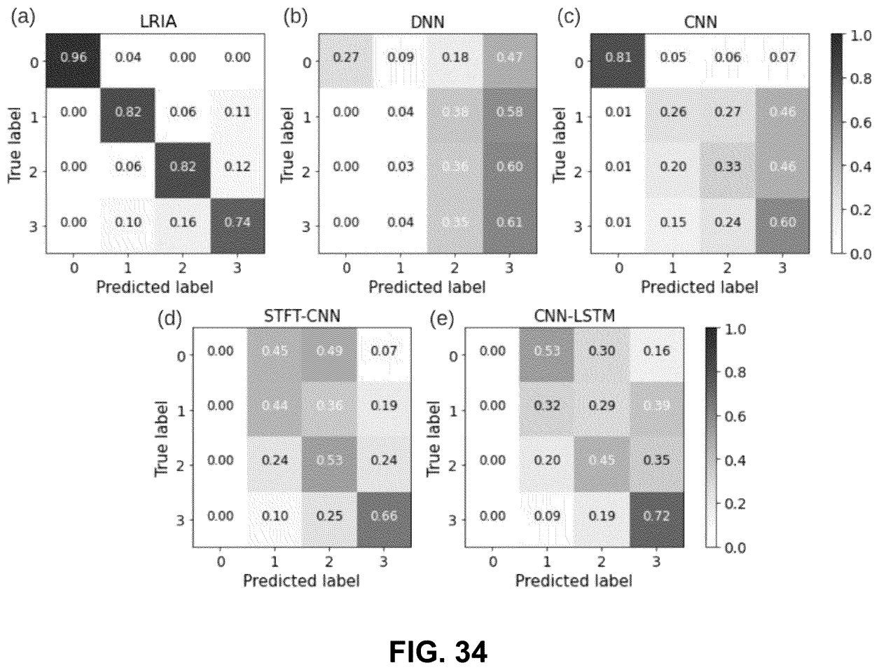

FIG. 34 is a plurality of confusion matrices results for five different models, including a LRIA model, DNN, CNN, STFT-CNN and a CNN-LSTM.

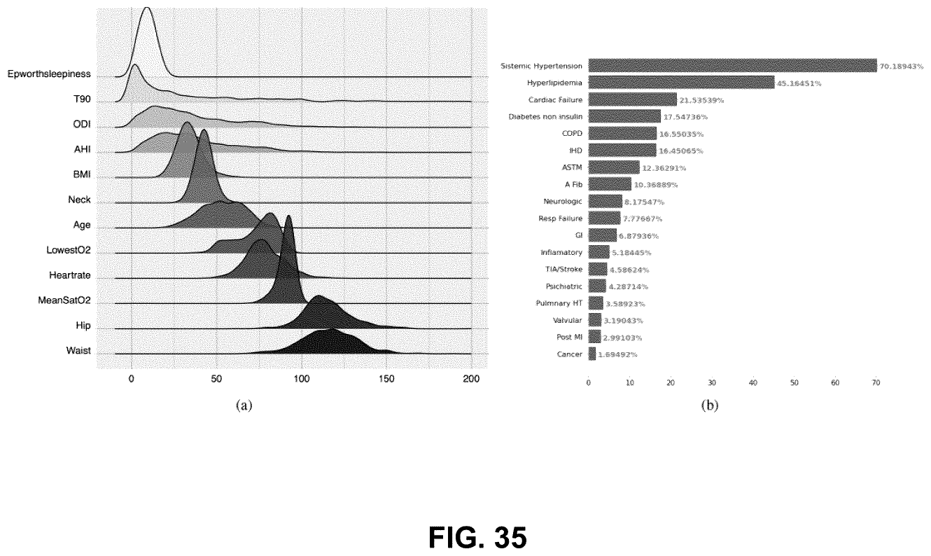

FIG. 35 is an illustration of key statistical information of a study cohort, including a density distribution plot of twelve physiological features and a bar chart demonstrating the medical history of the same cohort listed in descending order of percentage popularity.

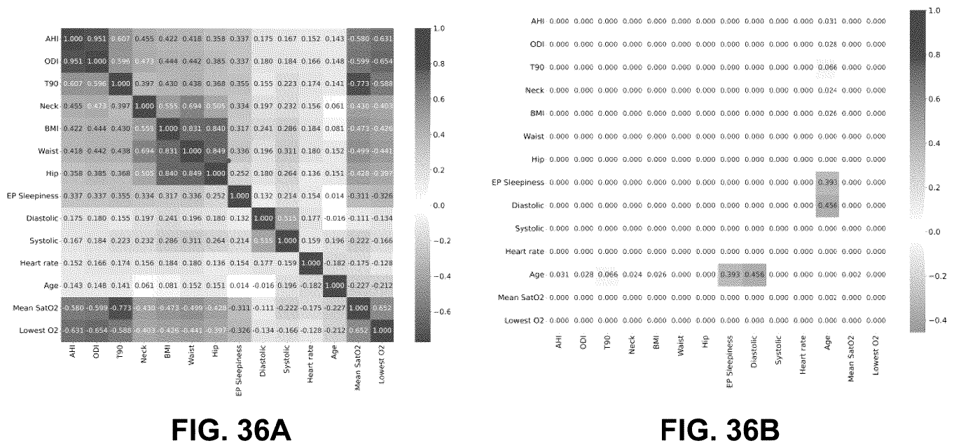

FIGS. 36A and 36B are non-linear matrix heatmaps indicating a plurality of statistical correlations between various physiological features in a study.

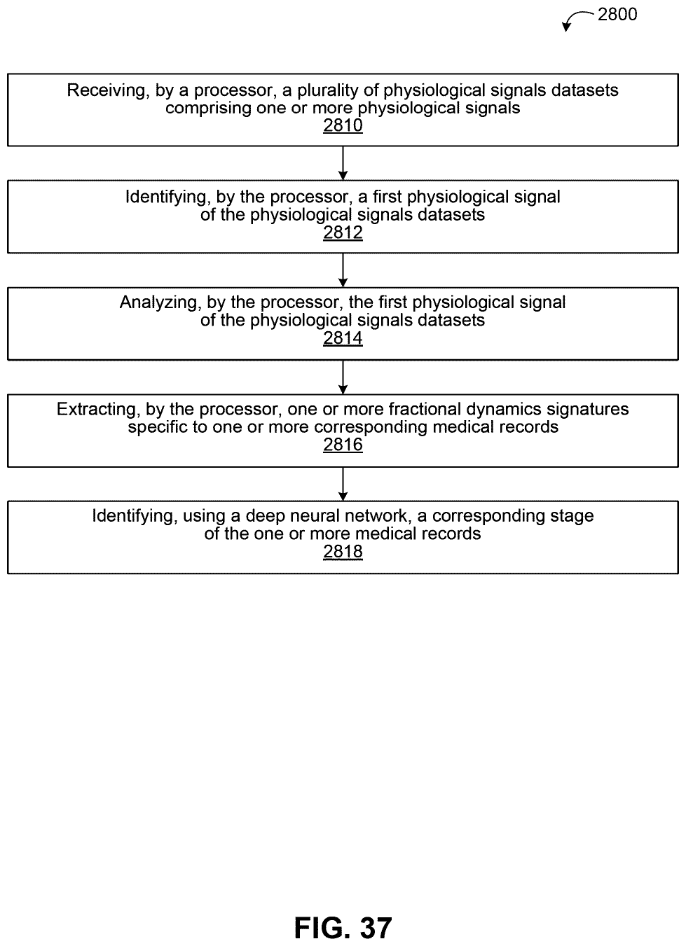

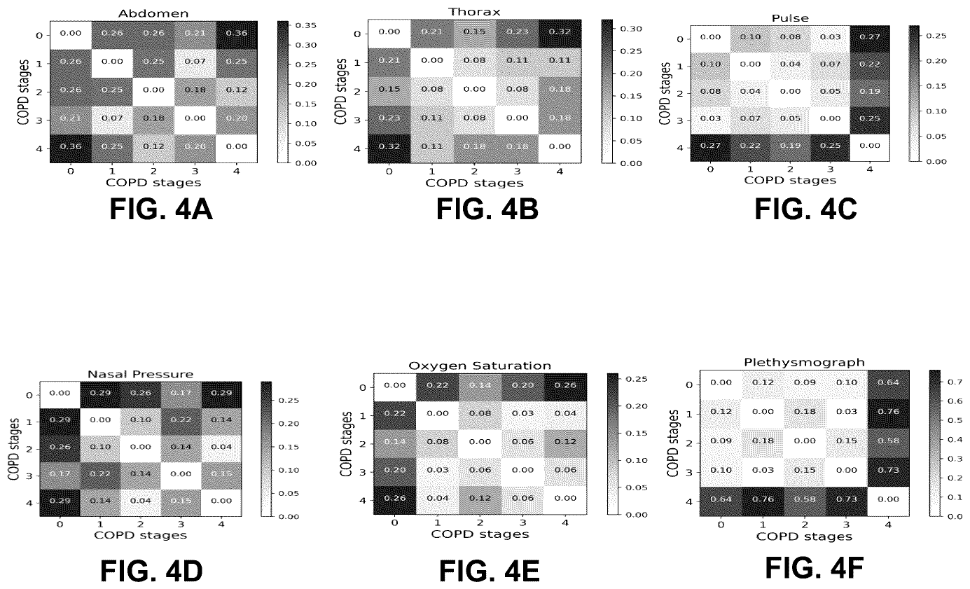

FIG. 37 is an illustration of a method of identifying a stage of one or more medical records using a deep neural network, according to some embodiments.

### DETAILED DESCRIPTION

The Global Initiative for Obstructive Pulmonary Disease (GOLD) defines COPD—based on pulmonary function testing or spirometry—as the ratio between the forced expiratory volume in one second and the forced vital capacity (FEV1/FVC) of <0.7 in a patient with symptoms of dyspnea, chronic cough, and sputum production, with an exposure history to cigarette smoke or biofuels, or occupational particulate matter. The spirometer is a device that measures the lung's volume and air debits, rendered as forced expiratory volume in one second (FEV1), forced vital capacity (FVC), and the ratio between FEV1 and FVC. Physicians use these parameters to classify patients in one of the following COPD stages: 1-Mild, 2-Moderate, 3-Severe, and 4-Very Severe. The almost unanimously accepted classification methodology is the COPD Gold Standard, although there are some differences in applying it.

Unfortunately, early COPD detection and diagnosis are challenging at the population level because relevant clinical signs are hard to detect in the early phases. When suspected, patients are ordinarily subjected to pulmonary function tests (i.e., spirometry) and mostly diagnosed when they are already in stages 2-4. Thus, designing therapies to improve the disease trajectory becomes difficult. Another problem with spirometry is that it does not always render reliable results, mainly when not performed in a specialized pulmonary center. Nonetheless, the fact that COPD has become a global threat further emphasizes the importance of decentralizing diagnosis, meaning that finding innovative methods to diagnose COPD outside respiratory medicine centers becomes paramount. Recent medical research suggests that personalized medicine could improve COPD diagnosis. One approach to COPD personalized care is identifying patient phenotypes based on comorbidities, simple clinical, and anthropometric data (e.g., age, body-mass index, smoker status). To this end, the medical practice uses two questionnaires to evaluate symptoms and evaluate the severity of the disease, namely COPD Assessment Test (CAT) and Medical Research Council Breathlessness Scale (MRC). Also, there are algorithmic methods for clustering COPD patients based on big data, complex network analysis, and deep learning. However, these techniques have not resulted in high prediction accuracy. The reason is that they only focus on investigating novel machine learning models rather than analyzing the geometric characteristics of the data. Furthermore, big data and Internet-of-Things (IoT) solutions were proven to be effective in COPD management, but such existent engineering systems are merely monitoring physiological signals to provide therapeutic feedback to physicians.

In the distribution of moment-wise estimates of the Hurst exponents in the healthy/COPD groups and offer a rigorous alternative to the conventional (spirometry-based) methodology for COPD diagnostics. Two hypotheses underpin the solutions. First, the physiological signals relevant to COPD (e.g., respiratory rate, oxygen saturation, abdomen breathing effort, etc.) have a multi-fractal nature, and their fractional-order dynamics specifically characterize the COPD pathogenic mechanisms. Second, the fingerprints of the COPD-related physiological processes with the coupling matrix can be captured in mathematical modeling of the physiological dynamics. In other words, the coupling matrix A deciphers the interdependencies and correlations between the recorded signals.

In some examples, two novel COPD physiological signals datasets (WestRo COPD dataset and WestRo Porti COPD dataset) are generated, and the relevant physiological signals recorded using one or more IoT devices (e.g., an IoMT (Internet of Medical Things) infrastructure) is analyzed. We extract the fractional dynamics signatures specific to the COPD medical records and train a deep neural network to diagnose COPD stages using both fractal dynamic network signatures and expert analysis (see FIGS. 1A-D).

FIGS. 1A, 1B, 1C, 1D, 1E, and 1F illustrate an example method for COPD stage prediction. FIG. 1A illustrates a system 100A (or a portion thereof) for identifying the physiological signals (e.g., via nasal cannula 103A, RIP belt 140A, RIP belt 142A, and pulse-oximeter 150A) with relevance in COPD and measure the same based on the medical observations 120A from the latest research in the field 110A. FIG. 1B illustrates a plurality of recordings of such physiological signals 100B with a medical sensor network, e.g., NOX T3™ portable sleep monitor. An example of a physiological signal 100C (Abdomen) recorded from a stage 4 COPD patient is shown in FIG. 1C. FIG. 1D (including scatter plot 100D and graph 100E) and 1E summarize the multifractal analysis in terms of scaling function and generalized Hurst exponent. In FIGS. 1C, 1D, and 1E, the analysis of the fractional-order dynamics is employed (e.g., according to the DNN architecture diagram 100F) to extract the signatures of the signals 110F, 120F as coupling matrices 130F and fractional-order exponents and use these signatures (along with expert diagnosis) to train a deep neural network 140F that can identify COPD stages 150F.

### Results

### Physiological Signals

### Recorded Signals

In a WestRo COPD dataset, each medical case includes of 12 signal records. First, we recorded seven physiological signals from our patients with the Respiratory Inductance Plethysmography—RIP (signals Thorax Breathing Effort and Abdomen Breathing Effort), the wireless pulse-oximeter (signals Oxygen Saturation Levels, SpO2 beat-to-beat mode, Pulse, and Plethysmograph), and the nasal cannula (signal Nasal Pressure). The NOX T3™ portable sleep monitor integrates and synchronizes the RIP, the wireless pulse-oximeter, and the nasal cannula. (Section Experimental, subsection Data collection provides detailed information). Moreover, the Noxturnal™ software application, which accompanies the NOX T3™ derived five additional signals: RIP Sum (the sum of the abdomen and thorax breathing effort signals), Activity (derived from the X, Y, and Z gravity axes), Position (in degrees, derived from the X, Y, and Z gravity axes, where the supine position is 0 degrees), Flow (derived from the nasal pressure signal), Resp Rate (respirations per minute derived from the RIP Sum signal). All the medical records in this dataset are gathered from four Pulmonology Clinics in Western Romania (Victor Babes Hospital—VB, Medicover 1—MD1, Medicover 2—MD2, and Cardio Prevent—CP clinics).

In a WestRo Porti COPD dataset, for example, 6 physiological signals are recorded in 13824 medical cases from 534 individuals during 2013-2020. The patients in the WestRo Porti are screened with the Porti SleepDoc 7 potable PSG device by recording 6 physiological signals (Flow, SpO2, Pulse, Pulse-wave, Thorax, Abdomen) overnight. The 6 Porti SleepDoc 7 signals correspond, respectively, to the following NOX T3 signals: Flow, Oxygen Saturation Levels, Pulse, Plethysmograph, Thorax Breathing Effort, and Abdomen Breathing Effort. The WestRo Porti COPD dataset is involved as an external dataset to validate the model and to test the robustness of the prediction and diagnosis approach where the medical signals records in this dataset interfere with another disease (sleep apnea).

### Fractal Properties of Physiological Signals

To verify the first hypothesis, in this section, the fractal features of raw signals (Thorax, Oxygen Saturation, Pulse, Plethysmograph, Nasal Pressure, and Abdomen) in healthy persons (stage 0) and critical COPD patients (stage 4) are shown in the WestRo COPD dataset. Detrended Fluctuation Analysis (DFA) is an effective method to investigate the statistical scaling and monofractal properties of non-stationary time series. For instance, the dichotomous models of fractional Gaussian noise (fGn) and non-stationary fractional Brownian motion (fBm)—initially described by Mandelbrot and van Ness—have been shown as a proper mono-fractal modeling framework for physiological signals. In addition, DFA is also widely used to investigate the time-series data in human respiration and heart rate. For instance, Peng et al. applied the DFA technique to quantify the scaling behavior of nonstationary respiratory time series and analyze the presence of long-range correlations of breathing dynamics in healthy adults. Furthermore, Schumann et al. used DFA to measure the autocorrelations in heartbeat intervals and respiration on longer time scales. To overcome the challenges of the ‘inversed’ singularity spectrum in standard multifractal analysis, Mukli, Nagy, and Eke proposed the focus-based multifractal formulas, which compute a moment-wise global-error parameter capturing the finite size effects and the signals' degree of multifractality. In order to mine the physiological complexity and account for its nonstationarity, we perform a comprehensive multifractal detrended fluctuation analysis (MF-DFA) of the collected data.

To analyze the fractional dynamic characteristics of the COPD physiological processes, the scaling (fluctuation) functions of the raw signals (Thorax, Oxygen Saturation, Pulse, Plethysmograph, Nasal Pressure, and Abdomen) in healthy people (stage 0) and very severe COPD patients (stage 4) (for detailed information about MF-DFA and the scaling function, see section Experimental, subsection Multifractal detrended fluctuation analysis) are calculated. FIGS. 2A, 2B, 2C and FIGS. 2G, 2H, and 2I, respectively, show the scaling functions calculated from Abdomen, Pulse, Plethysmograph, Thorax, Nasal pressure, and Oxygen saturation signals generated for a healthy person and panels (d-f) and (j-l) illustrate the scaling functions for the same signals from a very severe COPD patient (stage 4). The q values as q∈{−5, −3, −1, 1, 3, 5}.

FIGS. 2A, 2B, 2C, 2D, 2E, 2F, 2G, 2H, 2I, 2J, 2K, and 2L illustrate geometry of fluctuation profiles for the COPD-relevant physiological signals recorded from a normal abdomen and a stage 4 COPD abdomen. We calculate the scaling functions from 6 raw physiological signals:Abdomen, Thorax, Oxygen Saturation, Plethysmograph, Nasal Pressure, and Pulse, where the exponents are q∈[−5, 5]. Panels (a-c) and (g-i) with signals recorded from a healthy person, (d-f), and (j-l) with signals recorded from a representative stage 4 COPD patient. The resulting points (pink nodes) of the multifractal scaling function with power-law scaling will converge to a focus point (dark purple nodes) at the largest scale (L) if the tested signal has multifractal features.

In FIGS. 2A, 2B, 2C and FIGS. 2G, 2H, and 2I, the scaling functions under different q values will converge to a focus point (the dark purple nodes in each panel), except the Pulse signal. The focus point S(v, L) can be measured as the scaling function's fundamental property (for details onfocus point, see section Experimental, subsection Multifractal detrended fluctuation analysis). Accordingly, if a signal's scaling function has a focus point, it has multifractal features. The pink lines in FIGS. 2A-2L are the lines best-fitted to the scaling function's observed data and the pink dots represent different segmented sample sizes. To be more specific, we show the scaling functions of physiological signals extracted from all stage 4 patients and the healthy people (stage 0) in our dataset (where q∈[−5, 5]) in Supplementary material (for detailed information, see Supplementary material's section Hurst exponents of physiological signals). Hence, from FIGS. 2A, 2B, 2C and FIGS. 2G, 2H, and 2I, the raw signals—except the Pulse signal—in healthy persons have multifractal features. Conversely, in FIGS. 2D, 2E, 2F and FIGS. 2J, 2K, 2L, these scaling functions do not have focus points within the scale (except the Nasal pressure signal), which may suggest that such signals-recorded from severe COPD patients-do not have multifractal features.

FIGS. 3A, 3B, 3C, 3D, 3E, and 3F illustrate multifractal analysis of 6 physiological signals from healthy people (stage 0) and stage 4 COPD patients with 95% confidence interval: Generalized Hurst exponent H(q) as a function of q-th order moments (where q values are discretely extracted from −5 to 5) for physiological signals (Abdomen (a), Thorax (b), Oxygen Saturation (c), Plethysmograph (d), Nasal Pressure (e), and Thorax (f)) extracted from healthy people (stage 0) and severe COPD patients (stage 4).

FIGS. 3A, 3B, 3C, 3D, 3E, and 3F present the H(q) comparisons between physiological signals (Abdomen (a), Thorax (b), Oxygen Saturation (c), Plethysmograph (d), Nasal Pressure (e), and Thorax (f)) extracted from healthy people and patients with 95% confidence interval, where H(q) is the generalized Hurst exponent and represents the set of associated slopes of the pink lines in FIG. 2. As discussed in Bashan et al., the Hurst exponent measures the auto-correlations of the time-series data, where the autocorrelations are linked with both short-term and long-term memory [30], and larger Hurst exponent values (i.e., H>0.5) rep-resent persistent behavior (i.e., a more correlated structure) in monofractal fractional Gaussian noise type signals. In contract, the generalized Hurst exponent (H(q)) can capture the scaling properties of the time series data and reflects the heterogeneous scaling of the qth order moments of the increments of the temporal process [28]. H(q) confidence interval curves among healthy people (stage 0) and COPD patients (stage 4) in FIG. 3 are not fully overlapped, which shows that the physiological signals extracted from healthy people and severe COPD patients possess different fractional properties (The non-linear decrease distribution of the H(q) function illustrates that the fitted lines of scaling functions under different q values will converge to a focus point). In FIG. 3, all signals recorded from stage 4 COPD patients have different H(q) confidence intervals than the signals recorded from stage 0 participants, showing that signals collected from patients with different COPD stages have different fractional features. Consequently, the overarching conclusion of FIGS. 2A-2F and FIGS. 3A-3F is that the physiological signals with relevance for COPD have different fractional dynamics characteristics in healthy people—on the one hand—and severe COPD patients-on the other hand.

To analyze the H(q) functions across different COPD stages, we calculate the Wasserstein distance between each H(q) mean value curve in every physiological signal extracted from patients with different stages (we calculate the Wasserstein distances with the wasserstein_distance function in Python's scipy package). For detailed results about H(q) curves for all COPD stages, see Supplementary material's section Hurst exponents of physiological signals. The Wasserstein distance is a metric that measures differences between distributions.

FIGS. 4A, 4B, 4C, 4D, 4E, and 4F illustrate comparison of Wasserstein distance between the distributions of mean H(q) curves across different COPD stages (i.e., H(q) curves in FIG. 3, FIG. 11, and FIG. 12). Comparison in terms of Wasserstein distance between the H(q) distribution function for Abdomen (a), Thorax (b), Pulse (c), Nasal Pressure (d), Oxygen Saturation (e), and Plethysmograph (f) signals recorded from patients across all the COPD stages. In FIGS. 4A, 4B, 4C, 4D, 4E, and 4F, we show that, for each physiological signal, the signals' H(q) function extracted from stage 0 patients have the largest Wasserstein distance from stage 4 patients (except the Plethysmograph signal). In contrast, the H(q) functions of signals extracted from patients in moderate to severe COPD stages (i.e., 1, 2, and 3) have much smaller Wasserstein distances between each other (except the Abdomen signal). The results presented in FIGS. 4A, 4B, 4C, and 4D represent evidence that reinforces the medical observation stating that it is hard to distinguish early COPD stages. As such, it makes sense to analyze the spatial coupling between these physiological processes (signals) across time. These spatial coupling matrices A contain the different fractional features across different signal samples and can help us classify the signals recorded from suspected patients into different COPD stages.

The general conclusion in FIGS. 2A-2L, 3A-3F, and 4A-4F is that the physiological signals recorded gathered from healthy individuals and severe COPD patients have distinct fractal properties. This dichotomy is less evident for the Pulse signal, even if the tendency towards multifractality in healthy individuals is still present. The notable exception is the Nasal Pressure signal, which has multifractality in both healthy and COPD individuals. This observation suggests that COPD mostly affects the physiology of muscles involved in or supporting breathing (Thorax Breathing Effort and Abdomen Breathing Effort) and the circulatory system's physiology (reflected in Oxygen Saturation Levels and Plethysmograph). Conversely, the upper respiratory tract's physiological dynamics do not appear to be affected even by severe and very severe COPD stages.

### Fractional Dynamics Modeling of COPD Relevant Physiological Signals Subject to Unknown Perturbations

The dynamics of complex biological systems possess long-range memory (LRM) and fractal characteristics. For instance, several recent studies have demonstrated that stem cell division times, blood glucose dynamics, heart rate variability and brain-muscle interdependence activity are fitted by power-law distributions. The long short-term memory (LSTM) architecture is one of the most widely used deep learning approaches to analyze biological signals and perform prediction or classification. However, LSTM cannot fully represent the long memory effect in the input, nor can it generate long memory sequences from unknown noise inputs. Thus, when considering the very long-time series with long-range memory, LSTM cannot predict nor classify them with high accuracy. Indeed, in our study, the length of each physiological signal has more than 72000 data points. We aim to capture both short-range and long-range memory characteristics of various physiological processes and—at the same time—investigate the very long COPD signals with high accuracy. Therefore, we adopt the generalized mathematical modeling of the physiological dynamics,

$$\begin{matrix}{Δ}^{α} x [ k + 1 ] = Ax [ k ] + Bu [ k ]\end{matrix} \qquad ( 1 )$$

$$y [ k ] = Cx [ k ] ,$$

where x∈Rⁿ is the state of the biological system, u∈R^{p} is the unknown input and y∈Rⁿ is the output vector. The main benefits of this generalized mathematical representation are three-fold:

The model allows for capturing the intrinsic short-range memory and long-range memory of each physiological signal through either an integer or fractional order derivative. To connect the mathematical description with the discrete nature of measurements, the differential operator Δ is used as the discrete version of the derivative; for example, Δ¹ₓ[k]=x[k]−x[k−1]. A differential or-der of 1 has only one-step memory, and hence the classic linear-time invariant models are retrieved as particular cases of the adopted mathematical model. However, when the differential order is 1, the model cannot capture the long-range memory property of several physiological signals. Furthermore, we write the expansion of the fractional derivative and discretization [48] for any i^{th} state (1≤i≤n) as

$$\begin{matrix}{Δ}^{{α}_{i}} {x}_{i} [ k ] = {\sum }_{j = 0}^{k} ψ ( {α}_{i} , j ) {x}_{i} [ k - j ] ,\end{matrix} \qquad ( 2 )$$

where αᵢ is the fractional order corresponding to the i^{th} state and

$$ψ ( {α}_{i} , j ) = \frac{Γ ( i - {α}_{i} )}{Γ ( - {α}_{i} ) Γ ( j + 1 )}$$

with Γ(.) denoting the gamma function. Equation (2) shows that the fractional-order derivative framework provides a mathematical approach to capture the long-range memory by including all xᵢ[k−j] terms.

A modeling approach describes the system dynamics through a matrix tuple (α, A, B, C) of appropriate dimensions. The coupling matrix A represents the spatial coupling between the physiological processes across time, while the input coupling matrix B determines how the inputs affect these processes. We assume that the input size is always strictly smaller than the state vector's size, i.e., p<n. The coupling matrix A plays an essential role in deciphering the correlations between the recorded physiological signals. These correlations (entries of A) can indicate different physical conditions. For instance, when probing the brain electrical activity (through electroencephalogram (EEG) signals), the correlations can help at differentiating among various imaginary motor tasks [49]. Moreover, as described in this work, we can exploit these correlations to differentiate among pathophysiological states—such as degrees of disease progression—using physiological signals analysis. A key challenge is the estimation accuracy of these correlations (A matrix), notably for partially observed data. We have taken care of such limitations by using the concept of unknown unknowns introduced in reference.

Since we may have only partial observability of the complex biological systems, we take care of the unknown stimuli (excitations that may occur from other unobserved processes but cannot be probed); as such, we include in the model the vector variable u and study its impact on the recorded dynamics. In essence, we refer to this mathematical model as a multi-dimensional fractional-order linear dynamical model with unknown stimuli. The model parameters are estimated using an Expectation-Maximization (EM) based algorithm described in reference [38], to overcome the lack of perfect observability and deal with possibly small and corrupted measurements. In some examples, the algorithm is convergent and shows that it reduces modeling errors.

### Fractional Dynamics Deep Learning Prediction of COPD Stages

After extracting the signals' features (short-range and long-range memory) with the fractional dynamic mathematical model, we utilize these features (i.e., coupling matrices A) to train a deep neural network to predict patients' COPD stage. Deep learning is a machine learning approach that efficiently combines feature extraction and classification and it is a valuable tool for medical diagnosis (i.e., it can logically explain a patient's symptoms). We develop the fractional dynamics deep learning model (FDDLM) presented in this section to predict the COPD stages for our WestRo COPD dataset including of 4432 medical cases from patients in Pulmonology Clinics from Western Romania. We evaluate these cases and FDDLM by k-fold cross-validation and hold-out validation. K-fold cross-validation is a resampling procedure used to estimate how accurately a machine learning model will perform in practice. In k-fold cross-validation (k=5), we randomly shuffle the input dataset and split it into 5 disjoint subsets. We select each subset as the test set (20%) and combine the remaining subsets as the training set (80%). In hold-out validation, we hold one institution out at a time. I.e., we hold out data from one institution as a test set, and the remaining data from the other three institutions are used to train the models. The main steps of our approach are:

1. Constructing a COPD stage-predicting FDDLM, and calculate coupling matrix signatures (A) of relevant physiological signals (such as Thorax Breathing Effort or Abdomen Breathing Effort, etc.) to be used as the training data.

2. Training FDDLM with a training set to recognize the COPD level based on signal signatures.

3. Testing FDDLM with test set and predict patients' COPD stage.

The FDDLM uses a feedforward deep neural network architecture with four layers: one input layer, two hidden layers, and one output layer. FIGS. 5A and 5B illustrate an example of a network formulation of the FDDLM. FIG. 5A illustrates a basic structure of an artificial neuron model 500A. FIG. 5B illustrates the neural network model trained in FDDLM to identify COPD stages. The input layer takes the input 510A, 510B (i.e., signal signatures in the coupling matrix A) and pass it to hidden layers. From layer to layer, neurons compute the sum 530A, 530B of the weighted inputs 520A, 520B from the previous layer and settle the results through a nonlinear function 540A, 530B (activation function) as shown in FIG. 5A. In our FDDLM, hidden layers' activation functions are rectified linear unit (ReLU). The ReLU activation function 540A returns the input value when the input is ≤0 (otherwise, it returns 0), i.e., g(z)=max(0, z) for input value z. The output 550A layer's activation function is softmax (S), which normalizes the input values into a probability distribution. We utilized the rmsprop optimizer (with the default learning rate of 0.001) and the categorial cross entropy loss function. To avoid potential overfitting, we insert dropout function (with 0.8 keep rate) after each hidden layer to randomly select and ignore 20% of neurons.

### K-Fold Cross-Validation Results

All physiological signals are processed with the fractional dynamical model. Then, we feed the signal signatures from the coupling matrix A to FDDLM. We implement the neural network model in Python with Keras package and executed it on a computer with, for example, the Intel Core i7 2.2 GHz processor and 16 GB RAM. The table below illustrates the COPD stage predicting results for test set with FDDLM.

| COPD | Stage 0 | Stage 1 | Stage 2 | Stage 3 | Stage 4 |
|---|---|---|---|---|---|
| Sensitivity | 97.50% | 99.19% | 98.66% | 99.50% | 96.92% |
| Specificity | 99.87% | 99.61% | 99.41% | 99.31% | 99.88% |
| Precision | 99.15% | 97.60% | 99.20% | 98.06% | 98.44% |

We evaluate our results based on accuracy, sensitivity, loss, precision, specificity, and area under the receiver operating characteristic curve (AUROC). Our estimation of all results—generated from different models—uses the k-fold cross-validation method (with k=5). FIG. 6 presents our model's accuracy, AUROC, and loss curves on training and test sets. We also evaluate our model by comparing it with the Vanilla DNN and LSTM models trained on physiological signals (i.e., raw data) extracted from sleep monitors for reference. The Vanilla DNN and LSTM models have the same hyper-parameter setting as ours, including the optimizer, dropout configuration, loss function, activation functions, except the in-put layer size. We aim to choose the classic deep learning models with similar structures as baselines to investigate whether the fractional features (coupling matrix A) are easier for models to classify than the raw data. FIGS. 6 (a-c) show the training and test results observed from FDDLM. We observe that training and test accuracies and AUROC increase, while loss decreases during training.

FIGS. 6A, 6B, 6C, 6D, 6E, 6F, 6G, 6H, 6I, 6J, 6K, 6L show training and testing result comparisons (e.g., FIGS. 6A, 6D, and 6G illustrate accuracy, FIGS. 6B, 6E, and 6H illustrate AUROC, and FIGS. 6C, 6F, and 6I illustrate loss) of different deep learning models for the k-fold cross-validation. Training/testing accuracy (FIG. 6A), AUROC (FIG. 6B), and loss (FIG. 6C) for our FDDLM, where the training processes use signal signatures extracted with the fractional dynamic mathematical model. Training/testing accuracy, AUROC, and loss for the Vanilla DNN model (FIGS. 6D-6F) and the LSTM model (FIGS. 6G-6I), where the training processes use the physiological signals recorded with portable sleep monitors (raw data). Both Vanilla DNN and LSTM models share similar network structures and computation parameters with our FDDLM (Vanilla DNN has the same network structure as our model, except the input size). We obtain these results with the k-fold cross-validation (k=5). We also show the confusion matrices for test set across different models: FDDLM in panel (FIG. 6J), Vanilla DNN in (FIG. 6K), and LSTM in (FIG. 6L).

FIGS. 6D, 6E, 6F and FIGS. 6G, 6H, 6I present the training and test accuracy, AUROC, and loss results of Vanilla DNN model and LSTM model trained with physiological signals (raw data). The training and test results obtained from both Vanilla DNN model and LSTM model display overfitting, as test accuracies decrease (test loss increase) while training accuracies increase (training loss decrease). Thus, to deal with over-fitting, we involve the early-stopping technique in choosing the best-performing Vanilla DNN and LSTM. Compared to our FDDLM, the best-performing Vanilla DNN and LSTM result in much lower accuracies: 77.72%±0.688% and 78.54%±1.200%, respectively, while our FDDLM achieves 98.66%±0.447%.

FIGS. 6J, 6K, 6L) show the confusion matrix examples of FDDLM, Vanilla DNN, and LSTM, respectively. The confusion matrices present the prediction results of the test set. We construct the test set using the last 20% of data in the WestRo COPD dataset and train models with the first 80% of data.

The results point out that the FDDLM only misclassified 1.35% of the test sets in terms of individual COPD stages. Instead, Vanilla DNN and LSTM models misclassified 24.21% and 21.41% of the test sets, respectively. (We also investigated the possibility of using the convolutional neural network (CNN) model to characterize the physiological signal dynamics obtained from sleep monitors (raw data) and compare it with our FDDLM. The CNN model misclassified 63.87% of the test sets with k-fold cross-validation. For detailed information about CNN model and results, see section Experimental, subsection Neural network architecture for the WestRo COPD dataset and Supplementary material's subsection Training and testing results for CNN.) Table 1 presents the precision, sensitivity, and specificity of our model's predicting results; we find that all these results exhibit a substantial accuracy except the sensitivity of stage 4, which is 96.92%. In conclusion, our FDDLM predicts patients' COPD stages with a much higher accuracy than Vanilla DNN and LSTM models trained with physiological signals (raw data)—without overfitting—and represents an effective alternative to the spirometry-based diagnostic. (We performed the K-fold analysis of our model's accuracy both on a per-recording and a per-patient basis and obtained very similar results; see the Supplementary Information, section Per-patient based K-fold analysis.)

### Hold-Out Validation

The COPD dataset includes of physiological signals recorded from consecutive patients from four Pulmonology Clinics in Western Romania (Victor BabeşHospital—VB, Medicover 1—MD1, Medicover 2—MD2, and Cardio Prevent—CP clinics). To validate our FDDLM, we hold out all data extracted from a single institution as test set and train models on data recorded from the other three institutions. Following experimental setup from the previous section, we use Vanilla DNN and LSTM models as baselines with hyper-parameters similar to our FDDLM. FIG. 7A-7I shows the results of FDDLM, Vanilla DNN, and LSTM, in terms of accuracy, AUROC, and loss curves of training and test sets. We train Vanilla DNN and LSTM models on physiological signals (i.e., raw data). Conversely, we train our FDDLM on the fractional signatures extracted from the raw data.

FIGS. 7A, 7B, 7C, 7D, 7E, 7F, 7G, 7H, 7I show training and testing result comparisons of different deep learning models for the hold-out validation. The training/testing accuracy (FIG. 7A), AUROC (FIG. 7B), and loss (FIG. 7C) for the FDDLM, where the training processes use signal signatures extracted with the fractional dynamic mathematical model. The training/test accuracy (FIG. 7D) and (FIG. 7G), AUROC (FIG. 7E) and (FIG. 7H), and loss (FIG. 7F) and (FIG. 7I), for Vanilla DNN model and LSTM model, where the training processes use the physiological signals recorded with portable sleep monitors. Both Vanilla DNN and LSTM models share similar network structures with FDDLM (i.e., same neural network structure but different the input size). We obtain these results by holding out data from every single institution as the test set.

FIG. 7A-7I presents the training and test results (accuracy, AUROC, and loss) generated by FDDLM (FIGS. 7A-7C), Vanilla DNN model (FIG. 7D-FIG. 7F), and LSTM (FIG. 7G-FIG. 7I) model, respectively. FIG. 7A shows that the accuracy of FDDLM increases in training without overfitting. Conversely, the training and testing of Vanilla DNN and LSTM models clearly indicate lower values of the accuracy (80.73%±3.46% and 80.83%±3.67%, respectively) in FIGS. 7D and 7G, while FDDLM achieves 95.88%±1.76%.

Of note, we observe that the test accuracy under hold-out validation (95.88%) is lower than the accuracy obtained under k-fold cross-validation (for more detailed error analysis, we present the visualization of extracted features (embeddings) in the last hidden layer of FDDLM across k-fold and hold-out validation in the Supplementary materials subsection.) The reason for performance degradation in hold-out is that the data recorded from each medical institution are imbalanced. The Victor Babes (VB) and Cardio Prevent (CP) are two large clinics, and COPD patients are more willing to get diagnosis or medical treatment in large units or hospitals rather than small clinics, especially for severe and very severe COPD patients. Thus, the signals gathered from VB and CP are more comprehensive than the Medicover 1's (MD1) and Medicover 2's (MD2). In hold-out validation, although we balance the data across different institutions using over-sampling and under-sampling approaches, the remaining imbalance in data collection is still the leading cause of the prediction accuracy drop in the hold-out section.

FIGS. 8A, 8B, 8C, 8D, 8E, 8F, 8G, 8H, 8I, 8J, 8K, 8L illustrate the comparison of confusion matrices resulted from different deep learning models: fractional dynamics (FIGS. 8A-8D), Vanilla DNN (FIGS. 8E-8H), and LSTM (FIGS. 8I-8L). We built the test sets by holding out data gathered from one institution (i.e., VB, MD1, MD2, and CP) at a time. The matrix representations clearly show that our model outperforms both Vanilla DNN and LSTM—in all experiments and for all labels representing COPD stages—in terms of prediction errors.

FIGS. 8A, 8B, 8C, 8D, 8E, 8F, 8G, 8H, 8I, 8J, 8K, 8L show the confusion matrices (we only present the test set prediction results) for FDDLM, Vanilla DNN, and LSTM models by holding out each institution as the test set, respectively. Results shown in FIGS. 8A-8L prove that our model outperforms the baselines in pre-diction accuracy across the entire hold-out validation process. Especially for the early-stage detection (i.e., stages 1 and 2), our model achieves a higher accuracy than baselines (for the sensitivity, specificity, and precision of these confusion matrices, see Supplementary materials Table S1, Table S2, and Table S3). The reason is that, as opposed to Vanilla DNN and LSTM models, FDDLM can extract the signal signatures that contain the long-term memory of the time series. We also use the convolutional neural network (CNN) model to characterize the dynamics of the physiological signals recorded with sleep monitors to compare it with our FDDLM; the CNN model misclassified 64.49% of the test sets under hold-out validation. For detailed information about the CNN model and testing results, see section Experimental, subsection Neural network architecture for the WestRo COPD dataset and Supplementary material section Training and testing results for CNN).

In summary, our model outperforms all baselines in terms of prediction accuracy under both hold-out and k-fold cross-validation. The main conclusion is that FDDLM predicts patients' COPD stages with high accuracy and represents an efficient way to detect early COPD stages in suspected individuals. In-deed, such a low-invasive and convenient tool can help physicians make precise diagnoses and provide appropriate treatment plans for suspected patients.

### Transfer Learning

To evaluate our models' performance, we utilize the transfer learning mechanism to investigate the generalizability of our FDDLM. As such, we introduce the WestRo Porti COPD dataset. Transfer learning is a machine learning method that reuses a model designed for analyzing a dataset on another dataset, thus improving the learner from one domain by transferring information from another related domain.

The medical subjects in the WestRo Porti are consecutive individuals in the Victor Babes hospital records, screened for sleep apnea with the Porti SleepDoc 7 portable PSG device by recording 6 physiological signals; some individuals are also in various COPD stages. (For detailed information, see Experimental sub-section Data collection). The reasons for applying our COPD FDDLM are: (1) we want to verify that our model is valid on an external dataset; (2) we want to test our model's prediction performance when the medical signal records are interfered with by another disease (i.e., sleep apnea).

We test FDDLM with the WestRo Porti COPD dataset to check the prediction performance. Since the WestRo COPD Porti dataset only have 6 signals (whereas our model uses 12 signals), we reconstructed the input size of the models from 144×1 to 36×1, retrained a new FDDLM with WestRo and tested it on the WestRo Porti COPD dataset to check the performance. (Note that the WestRo Porti COPD dataset patients are not included in the WestRo COPD dataset). The prediction accuracy of FDDLM is 90.13%±0.89% with fine-tuning. The explanation for the accuracy drop is that (i) the models are previously designed for analyzing medical records with 12 signals, not 6; (ii) the two datasets are recorded by two different portable devices having different frequencies, which influences the convergence of the coupling matrices; (iii) the co-existed sleep apnea in the medical records gathered from the WestRo Porti COPD dataset also influence the prediction performance.

### SUMMARY

Nowadays, DNN and LSTM models are the two popular deep learning models in analyzing and classifying time-series data. However, they do not present high-performance when the time series are precisely long (the reason is that the current model cannot correctly extract the long-term memory from very long time series). In this work, we developed a novel fractional dynamics-based model which can appropriately analyze long-term memory from COPD physiological signals datasets by extracting fractional features (coupling matrix A) from very long time-series data. The extracted fractional features are more straightforward for deep learning models to classify than the raw signals, and even the linear classifier achieves a good accuracy (for detailed information, see “Supplementary materials” subsection “Linear classifier of coupling matrices”). Therefore, based on the results shown in k-fold cross-validation, hold-out validation, and external validations, we conclude that our FDDLM has enough generalizability to be applied to different kinds of COPD records that contain physiological signals. Indeed, based on the transfer learning results, we argue that our FDDLM is robust enough to predict COPD stages across different datasets with high accuracy.

Besides the high accuracy of our COPD stage prediction method, we made a deeper analysis of the cases where our predictions failed. Overall, we have 19 misclassified cases (out of 534); most of them (i.e., 9) correspond to borderline cases where the spirometry values are exactly (or very close to) threshold values between stages. Some borderline COPD cases also overlap with other respiratory diseases, such as sleep apnea or asthma (3 cases), while one borderline case overlaps with heavy smoking. We also found that 7 misclassified cases overlap with comorbidities, such as severe sleep apnea, asthma, or obesity. For 3 cases, there is no apparent explanation for the misclassification, although one of them is a heavy smoker; in all these 3 cases, the individuals have no COPD but are predicted at stage 2. Another finding is that the noise in physiological signals recordings may rarely cause misclassifications of non-COPD cases (i.e., stage 0), as we noticed in 3 cases (in our datasets, we have 143 patients with stage 0).

The overarching conclusion is that comorbidities (especially sleep apnea and asthma) can alter the physiological signals to affect the prediction accuracy, mainly when dealing with borderline COPD stages.

Future works have to consider the comorbidity cases carefully. Indeed, more people in the aging population suffer from multi-morbidity, defined as two or more chronic conditions. COPD is common in multi-morbid patients, and many patients with COPD present concomitant other obstructive diseases, such as obstructive sleep apnea (OSA) and asthma, due to an increased prevalence of obesity, smoking, and allergy in the general population. Recent estimation of OSA prevalence shows almost 1 billion people affected, with prevalence exceeding 50% in some countries. Moreover, around 300 million people have asthma worldwide, and it is likely that by 2025 a further 100 million may be affected.

### Discussion

COPD is often a silent and late-diagnosed disease, affecting over 300 million people worldwide; intrinsically, its early discovery and treatment are crucial for the patient's quality of life and—ultimately—survival.

The inception of a disease entails a preclinical period where it is asymptomatic and—perhaps—reversible. Ideally, this period includes very early events that can occur even before birth. Early COPD stages do not exhibit evident clinical signs; therefore, conventional spirometry-based diagnosis becomes improbable. However, the development of biomarkers that include detecting genetic variants for COPD development's susceptibility is a priority. The COPD onset is a phase of early COPD where the disease may express itself with some symptoms, including a minimal airflow limitation. In this phase, spirometry is insufficient to attain a reliable diagnosis, which calls for new COPD detection tools.

The current strategy of waiting for surfacing symptoms to signal the disease presence is not efficient if we want to impact COPD's natural course. Targeting early COPD stages in younger individuals could identify those susceptible to rapid disease progression, leading to novel therapies to alter that progression. New validated biomarkers (other than spirometry) of different lung function trajectories will be essential for the design of future COPD prevention and treatment trials. Indeed, spirometry with FEV1 may not be the most sensitive test and may have particular limitations in identifying the early COPD stages. Moreover, impulse oscillometry and specific airway conductance were able to identify more subtle changes in lung function than traditional spirometry. Impulse oscillometry can identify abnormalities in patients who report COPD symptoms but do not have abnormal spirometry. Such complementary diagnostic modalities could potentially aid in the early recognition of COPD, especially those whose symptoms do not match their spirometry results.

The adjustment of current diagnostic approaches and the adoption of alternative modalities may allow for earlier identification of COPD patients. The period of the most rapid decline in lung function occurs early, and during this period, different testing strategies, smoking cessation efforts, and the initiation of treatment may be most beneficial.

This work proposes an alternative, precise diagnostic approach to overcome the conventional (spirometry-based) method's limitations by using a fractional dynamics deep learning methodology. We involved the fractional-order dynamics model in extracting the signatures from the physiological signals (recorded by a medical sensor network from suspected patients) and trained a neural network model with these signal signatures to make the COPD stage prediction. From a clinical standpoint, our fluctuation profile analysis for physiological signals with relevance in COPD (see FIGS. 2A-2L) shows that multifractality is the fingerprint of healthiness—in healthy people, physiological signals present both short and long-range memory. Conversely, monofractals or narrow-spectrum multifractals indicate a medical condition. We noticed two exceptions to this observation; first, the Pulse signal is narrow-spectrum multifractal even in non-COPD (i.e., stage 0) individuals; second, the Nasal Pressure signal is multifractal in both COPD and non-COPD subjects. The possible explanation for the fact that the Pulse signal is not entirely multifractal in non-COPD individuals is that—most probably—such subjects may have other medical conditions, such as sleep apnea or cardiovascular problems, which (along with the associated medication) altered the short and long-range properties of the signal. (Indeed, all patients were referred for polysomnography because of suspicion of respiratory disorders; some turned out to be COPD-free, yet most of them have other respiratory disorders, such as sleep apnea, as well as cardiovascular conditions.) The Nasal Pres-sure signal is multifractal in both COPD and non-COPD subjects because it manifests upper airway dynamics, which may be less affected as COPD is an inflammation (resulting in the narrowing) of the lung airways.

We confirm the results with k-fold cross-validation and hold-out validation and show that our approach can predict the patients' COPD stages with high accuracy (98.66% 0.45%). The accuracy is particularly high for COPD stages 1-3, suggesting that our method is distinctly efficient for detecting early-stage COPD. Furthermore, based on the transfer learning validation, we prove that our model can also achieve high prediction accuracy when the medical signal records are interfered with by another disease (i.e., sleep apnea). Our work makes two main contributions in medical diagnosis and machine learning fields. First, our fractional dynamics deep learning model makes a precise and robust COPD stage prediction that can work before the disease onset, making it especially relevant for primary care in remote areas or geographical regions missing medical experts, where the vast majority of patients with early and mild COPD are diagnosed and treated (Although the fractional-order dynamic model performs well in diagnosing COPD, it may not be generalized in investigating other physiological signals. Indeed, not all physiological signals have multifractal features (e.g., Ivanov et al. showed that the human gait interstride interval time series among healthy people do not show multifractality)). Second, we developed a valid fractional deep learning approach that outperforms the traditional deep learning model (e.g., DNN, LSTM, CNN) of classifying and analyzing very long time-series raw data. (We provide de-tailed information to explain why our model can efficiently reduce the learning complexity and achieve a high prediction accuracy in section Experimental, subsection Mutual information analysis).

Nowadays, the conventional spirometry-based diagnosis is the dominating approach to diagnosing COPD. The problem is that it entails many error-prone steps/stages involving human intervention such that general practitioners or well-trained nurses may also misdiagnose suspected patients (of the 4610 sub-jects, 96.5% had a valid screening spirometry test [64]). Such a result emphasizes that training and technique reinforcement are paramount, yet many primary care units do not have the resources to perform them. In this paper, our fractal dynamics deep learning method eliminates human intervention (and error) as much as possible; any nurse or MD can place the sensors on the patient's body, turn on the NOX device to record the physiological signals in its local memory. Afterward, we are dealing with a completely automated, computer-based process. The sufficient signal length required for a correct diagnostic is 10 minutes (for detailed information, see Supplementary material, section Convergence of coupling matrix). Therefore, our method is simple, robust, requires little human intervention, and has a relatively small duration of physiological signal records; this also makes it suitable for addressing critical social aspects of healthcare. First, there is equal opportunity in accessing reliable medical consultation for COPD, especially in areas with a lower socioeconomic status where people do not have the means to travel to a specialized state-of-the-art respiratory clinic. With our method, any medical mission in such an area can efficiently record data from many individuals in need and then process it automatically. Second, our method abides by the commandments of universal health care amid the COVID-19 pandemic, as it filters most of the physical interaction entailed by regular spirometry [16]. Although the MF-DFA methods we use are widely used, they cannot exclude that the focus points are due to bimodality, multifractal noise, or mere monofractality. Hence, in future work, we plan to employ the robust multifractal analysis developed to analyze the physiological signals and develop a new machine-learning framework to improve the robustness of predicting COPD stages.

### Experimental Section

### Data Collection

WestRo COPD dataset: The study cohort represents consecutive patients from 4 Pulmonology Clinics in Western Romania (i.e., the WestRo cohort, comprising patients from Victor Babeş—VB, Medicover 1—MD1, Medicover 2—MD2, and Cardio Prevent—CP clinics). Data include of physiological signals recorded over long periods (i.e., 6-24 hours), using a protocol that ensures complete patient privacy. To obtain a reliable medical diagnostic for each patient, we also collected the following data records: age, sex, body mass index (BMI, as a ratio between mass in kilograms and the squared value of height in meters), smoking history (in years since quitting smoking, with value 0 representing current smokers), FVC and FEV1 in liters and percentage (used to render the COPD stage diagnosis according to the ERS/ATS recommendation, with stage 0 representing no COPD), COPD assessment test (CAT) and dyspnea severity with modified Medical Research Scale (MRC) questionnaires, exacerbations (number of moderate to severe exacerbation in the last year), COPD onset (number of years since the onset). For detailed information about CAT and MRC, see Supplementary material, section Standard questionnaires, exacerbation history, and comorbidities of COPD patients.

Table 2 below illustrates information of all the COPD patients in our dataset which include medical center; COPD stage; COPD onset; age; gender; smoking status; body mass index (BMI); standard questionnaires (CAT—COPD assessment test, mMRC—modified Medical Research Council dyspnea scale), exacerbation history, and comorbidities (cardiometabolic (CC), cancer (CA), metabolic (MC), psychiatric (PC), and renal (RD)).

| Patients | | COPD | COPD | | | Smoking | | | | | | | | | |
|---|---|---|---|---|---|---|---|---|---|---|---|---|---|---|---|
| ID | Center | stage | onset | Age | Gender | status | BMI | CAT | MRC | Exacerbation | CC | CA | MC | PC | RD |
| P1 | CP | 2 | 0 | 66 | M | Ex 7 | 43.27 | 26 | 3 | 1 | 1 | 0 | 1 | 0 | 1 |
| P2 | CP | 2 | 1 | 63 | F | Smoker | 48.49 | 33 | 3 | 1 | 1 | 0 | 1 | 0 | 0 |
| P3 | CP | 2 | 0 | 43 | M | Smoker | 33.58 | 24 | 3 | 0 | 1 | 0 | 0 | 0 | 0 |
| P4 | CP | 2 | 7 | 71 | M | Ex 11 | 27.21 | 22 | 2 | 0 | 1 | 0 | 1 | 0 | 0 |
| P5 | CP | 3 | 0 | 63 | M | Ex 15 | 47.12 | 22 | 2 | 1 | 1 | 0 | 1 | 0 | 0 |
| P6 | VB | 3 | 7 | 70 | M | Ex 12 | 27.31 | 22 | 2 | 1 | 1 | 0 | 0 | 0 | 0 |
| P7 | MD1 | 2 | 4 | 72 | M | Ex 23 | 68.17 | 24 | 3 | 1 | 1 | 0 | 1 | 0 | 0 |
| P8 | VB | 2 | 2 | 88 | M | Ex 32 | 42.97 | 32 | 3 | 1 | 1 | 0 | 1 | 0 | 1 |
| P9 | CP | 2 | 12 | 54 | M | Smoker | 21.91 | 23 | 2 | 2 | 1 | 0 | 0 | 0 | 0 |
| P10 | MD2 | 2 | 0 | 67 | M | Ex 10 | 31.14 | 21 | 3 | 0 | 1 | 0 | 1 | 0 | 0 |
| P11 | CP | 2 | 0 | 66 | M | Ex 12 | 48.45 | 32 | 3 | 1 | 1 | 0 | 1 | 0 | 1 |
| P12 | VB | 3 | 5 | 76 | M | Ex 8 | 28.91 | 34 | 4 | 1 | 1 | 0 | 1 | 0 | 1 |
| P13 | CP | 3 | 7 | 70 | M | Ex 25 | 36.57 | 26 | 3 | 0 | 1 | 0 | 0 | 0 | 0 |
| P14 | MD1 | 3 | 1 | 64 | M | ex 24 | 26.5 | 15 | 2 | 0 | 1 | 0 | 0 | 0 | 0 |
| P15 | VB | 2 | 5 | 59 | M | Smoker | 38.1 | 31 | 3 | 1 | 1 | 0 | 1 | 0 | 0 |
| P16 | VB | 1 | 1 | 48 | F | ex 15 | 28.4 | 13 | 2 | 1 | 0 | 0 | 1 | 0 | 0 |
| P17 | CP | 1 | 2 | 54 | M | ex 10 | 26.3 | 11 | 2 | 0 | 1 | 1 | 1 | 0 | 0 |
| P18 | VB | 1 | 1 | 61 | M | ex 21 | 31.5 | 13 | 2 | 1 | 1 | 0 | 1 | 0 | 0 |
| P19 | CP | 2 | 0 | 62 | M | Smoker | 23.59 | 16 | 2 | 0 | 1 | 0 | 0 | 0 | 0 |
| P20 | VB | 2 | 0 | 63 | M | Smoker | 32.81 | 14 | 2 | 1 | 1 | 0 | 0 | 0 | 0 |
| P21 | VB | 4 | 5 | 64 | M | Smoker | 32.87 | 28 | 3 | 2 | 1 | 1 | 0 | 0 | 0 |
| P22 | CP | 2 | 0 | 76 | M | Smoker | 35.43 | 36 | 4 | 1 | 1 | 0 | 0 | 0 | 0 |
| P23 | VB | 1 | 2 | 69 | M | Ex 22 | 37.35 | 28 | 3 | 0 | 1 | 0 | 1 | 0 | 0 |
| P24 | CP | 2 | 1 | 70 | F | no | 44.1 | 25 | 2 | 1 | 1 | 0 | 1 | 0 | 0 |
| P25 | VB | 3 | 0 | 72 | M | Ex 10 | 39.86 | 36 | 4 | 1 | 1 | 0 | 1 | 0 | 1 |
| P26 | MD2 | 3 | 1 | 65 | M | Ex 1 | 28.4 | 24 | 2 | 0 | 1 | 0 | 1 | 0 | 1 |
| P27 | CP | 1 | 3 | 56 | M | ex 5 | 34.1 | 19 | 2 | 0 | 0 | 0 | 1 | 0 | 0 |
| P28 | CP | 3 | 0 | 75 | M | Smoker | 23.67 | 32 | 3 | 1 | 1 | 0 | 0 | 0 | 0 |
| P29 | VB | 3 | 3 | 60 | M | Smoker | 22.15 | 21 | 3 | 0 | 0 | 0 | 0 | 1 | 0 |
| P30 | CP | 3 | 2 | 55 | F | Smoker | 39.1 | 27 | 2 | 2 | 1 | 0 | 0 | 0 | 0 |
| P31 | MD2 | 4 | 6 | 67 | M | Ex 2 | 37.18 | 34 | 4 | 1 | 1 | 1 | 1 | 1 | 0 |
| P32 | CP | 4 | 7 | 64 | M | Ex 4 | 33.26 | 28 | 3 | 0 | 1 | 0 | 1 | 0 | 0 |
| P33 | VB | 2 | 3 | 58 | M | Smoker | 27.08 | 26 | 3 | 1 | 1 | 0 | 0 | 1 | 0 |
| P34 | CP | 2 | 3 | 53 | M | Ex 38 | 29.67 | 16 | 2 | 1 | 1 | 0 | 0 | 0 | 0 |
| P35 | VB | 3 | 1 | 76 | M | Ex 6 | 36.39 | 32 | 3 | 1 | 1 | 0 | 0 | 0 | 0 |
| P36 | CP | 4 | 8 | 40 | M | no | 41.5 | 35 | 3 | 2 | 1 | 0 | 0 | 1 | 0 |
| P37 | CP | 2 | 0 | 77 | M | Ex 23 | 38.06 | 26 | 3 | 0 | 1 | 0 | 1 | 0 | 0 |
| P38 | MD1 | 2 | 1 | 49 | F | no | 40.2 | 16 | 2 | 0 | 0 | 0 | 0 | 0 | 0 |
| P39 | CP | 2 | 0 | 67 | M | Ex 7 | 22.89 | 28 | 3 | 1 | 1 | 0 | 1 | 0 | 0 |
| P40 | CP | 3 | 8 | 68 | F | Ex 7 | 22.86 | 28 | 3 | 3 | 1 | 0 | 0 | 0 | 0 |
| P41 | VB | 2 | 3 | 72 | M | Ex 20 | 31.05 | 19 | 2 | 1 | 1 | 0 | 1 | 1 | 0 |
| P42 | CP | 3 | 12 | 67 | M | Ex 9 | 30.42 | 29 | 2 | 1 | 1 | 0 | 0 | 0 | 0 |
| P43 | CP | 3 | 0 | 68 | M | Smoker | 33.95 | 34 | 3 | 1 | 1 | 1 | 1 | 0 | 0 |
| P44 | VB | 4 | 1 | 65 | F | Smoker | 32.46 | 18 | 2 | 0 | 1 | 0 | 0 | 0 | 0 |
| P45 | VB | 2 | 3 | 56 | M | Smoker | 18.62 | 12 | 2 | 2 | 0 | 0 | 0 | 0 | 0 |
| P46 | VB | 2 | 2 | 43 | M | no | 31 | 17 | 2 | 1 | 0 | 0 | 1 | 0 | 0 |
| P47 | CP | 2 | 1 | 55 | F | ex 10 | 28.5 | 20 | 2 | 0 | 1 | 0 | 0 | 0 | 0 |
| P48 | CP | 0 | 0 | 65 | M | no | 33 | 0 | 0 | 0 | 0 | 0 | 0 | 0 | 0 |
| P49 | MD1 | 0 | 0 | 43 | M | no | 35.9 | 0 | 0 | 0 | 0 | 0 | 0 | 0 | 0 |
| P50 | MD2 | 0 | 0 | 68 | M | ex 24 | 31.4 | 0 | 0 | 0 | 0 | 0 | 0 | 0 | 0 |
| P51 | MD1 | 0 | 0 | 46 | M | no | 31.3 | 0 | 0 | 0 | 0 | 0 | 0 | 0 | 0 |
| P52 | MD1 | 0 | 0 | 51 | M | no | 25.4 | 0 | 0 | 0 | 0 | 0 | 0 | 0 | 0 |
| P53 | VB | 0 | 0 | 72 | F | Smoker | 27.4 | 0 | 0 | 0 | 0 | 0 | 0 | 0 | 0 |
| P54 | MD2 | 0 | 0 | 57 | M | ex 15 | 29.6 | 0 | 0 | 0 | 0 | 0 | 0 | 0 | 0 |

We also provide all data about body mass index (BMI), COPD onset, standard questionnaires (CAT—COPD assessment test, mMRC—modified Medical Research Council dyspnea scale), exacerbation history, and comorbidities (cardiometabolic, cancer, metabolic, psychiatric, renal) for all the patients in our dataset in Table 2.

WestRo Porti COPD dataset: The WestRo Porti cohort includes of polysomnography (PSG) physiological signals recorded in 13824 medical cases from 534 individuals during 2013-2020. The subjects in the WestRo Porti are consecutive individuals in the Victor Babes hospital records, screened for sleep apnea with the Porti SleepDoc 7 portable PSG device by recording 6 physiological signals (Flow, SpO2, Pulse, Pulsewave, Thorax, Abdomen) overnight, during sleep. The 6 Porti SleepDoc 7 signals correspond, respectively, to the following NOX T3 signals: Flow, Oxygen Saturation Levels, Pulse, Plethysmograph, Thorax Breathing Effort, and Abdomen Breathing Effort.

In this work, the same medical doctor gave all diagnoses that led to determining the COPD labels across all institutions. Moreover, the medical doctor used the same devices and diagnosis method (sensors to collect physiological signals from patients and spirometers). In addition, the same medical doctor collected the data in all clinics; spirometry was conducted with the help of trained, experienced technicians, certified in pulmonary function testing, following the ATS/ERS protocol (American Thoracic Society/European Respiratory Society). In all clinics included in our study, there is a quality control program for all procedures.

Spirometry quality assurance includes examining test values and evaluating both the volume-time and flow-volume curves for evidence of technical errors. During testing, technicians record a valid test com-posed of at least 3 acceptable maneuvers with includeent (i.e., repeatable) results for FVC and FEV1. Achieving repeatability during testing means that the differences between the largest and second-largest values for both FVC and FEV1 are within 150 ml. Additional maneuvers can be attempted—up to a maximum of 8—to meet these criteria for a valid test. The observer bias is reduced by ensuring that observers are well trained (specialized clinics do that regularly with certification diplomas), having clear rules and procedures in place for the experiment (i.e., the ERS/ATS protocol), and ensuring that behaviors are clearly defined. Therefore, since the same medical doctor performed all evaluations with the same equipment and diagnosis approach, we are confident that we substantially mitigated the intra- and inter-observer variability.

### Multifractal Detrended Fluctuation Analysis

Multifractal detrended fluctuation analysis (MF-DFA) is an effective approach to estimate the multifractal properties of biomedical signals [28]. The first step of MF-DFA is to calculate the cumulative profile (Y (t)):

$$Y ( t ) = {\sum }_{i = 1}^{t} X ( i ) - 〈 X ( i ) 〉 , \qquad ( 3 )$$

where X is a bounded time series. Then, divide the cumulated signal equally into Nₛ non-overlapping time windows with length s, and remove the local line trend (local least-squares straight-line fit) yᵥ from each time window. Therefore, F(v, s) characterizes the root-mean-square deviation from the trend (i.e., the fluctuation),

$$F ( v , s ) = \sqrt{\frac{1}{s} {\sum }_{i = 1}^{s} {\{ Y [ ( v - 1 ) s + i ] - {y}_{u} ( i ) \}}^{2} .} \qquad ( 4 )$$

In some examples, the scaling function as:

$$S ( q , s ) = {\{ \frac{1}{N} {\sum }_{v = 1}^{{N}_{s}} μ {( v , s )}^{q} \}}^{1 / q} , \qquad ( 5 )$$

where μ is an appropriate measure which depends on the scale of the observation (s). Hence, the scaling function is defined by substituting equation 4 into equation 5,

$${S}_{F} ( q , s ) = {\{ \frac{1}{{N}_{s}} {\sum }_{v = 1}^{{N}_{s}} {\{ \frac{1}{s} {\sum }_{i = 1}^{s} {\{ Y [ ( v - 1 ) s + i ] - {y}_{u} ( i ) \}}^{2} \}}^{q / 2} \}}^{1 / q} . \qquad ( 6 )$$

The moment-wise scaling functions for a multifractal signal exhibit a convergent structure that yields to a focus point for all q-values. Focus points can be deduced from equation 5, by considering the signal length as a scale parameter:

where the value of μ represents the entire signal, namely N_{L}=1 (i.e., takes only one time

$$S ( q , L ) = {\{ \frac{1}{{N}_{s}} {\sum }_{v = 1}^{{N}_{L}} μ {( v , L )}^{q} \}}^{1 / q} = {\{ μ {( v , L )}^{q} \}}^{1 / q} = μ ( v , L ) , \qquad ( 7 )$$

window into consideration). According to equation 7, the scaling function S(q, L) becomes independent from the exponent q and the moment-wise scaling functions will converge to μ(v, L) which is the mathematical definition of the focus point.

### Neural Network Architecture for the WestRo COPD Dataset

Fractional dynamics deep learning model (FDDLM). In our work, FDDLM includes of two parts: (1) fractional signature extraction (for more details, please see section Experimental, subsection Multi-fractal detrended fluctuation analysis) and (2) a deep learning model. Keeping in mind the input size of our training data (i.e., the coupling matrix A) and available GPU computational power, we constructed a deep neural network (DNN) architecture to handle the training and prediction progress. We built the network with the TensorFlow Python framework. Our deep neural network includes of 6 layers: 1 in-put layer, 2 hidden layers, 2 dropout layers, and 1 output layer. Also, we resampled the input data (matrix A) to 144×1 voxels and normalized each value within the range [0, 1](normalization is a technique for training deep neural networks that standardizes the inputs to a layer). We placed the dropout layers after each hidden layer with a 20% drop rate (the first hidden layer has 300 neurons and the second hidden layer has 100 neurons); each fully connected hidden layer utilizes the ReLU activation function. The softmax is utilized as the activation function in the output layer. The DNN is optimized with the rmsprop optimizer with a learning rate of 0.0001 and trained with the cross entropy loss function. FD-DLM is trained over 500 epochs with a batch size of 64 samples. Overall, the number of trainable parameters of the deep learning model is 74,105.

Vanilla deep neural network (DNN) model. The Vanilla DNN model shares the same network structure with the deep learning model in our FDDLM, except the input layer. The Vanilla DNN contains 6 layers: 1 input layer (the input data, namely, the physiological signals are reshaped to 72000×1 voxels, and each value is normalized within the range [0, 1]), 2 hidden layers (the first hidden layer has 300 neurons and the second hidden layer has 100 neurons), 2 dropout layers, and 1 output layer. The activation function for each fully connected hidden layer is ReLU, and the activation function for the output layer is softmax. The Vanilla DNN model is optimized with the rmsprop optimizer, having a default learning rate of 0.0001, and trained with the cross entropy loss function. The model is trained over 500 epochs with a batch size of 64 samples. The total number of trainable parameters of the Vanilla DNN model is 21,630,905.

Long short-term memory (LSTM) model. The LSTM model in this work has the following layers: an input layer (the input physiological signals are reshaped to 6000×12 voxels, and each value is normalized within the interval [0, 1]), an LSTM layer (with 300 neurons), a dropout layer (with a 0.2 dropout rate), a dense layer (with 100 neurons), a dropout layer (with a 0.2 dropout rate), and an output layer. ReLU is the activation function for the LSTM and dense layers. The model is optimized with rmsprop having a default learning rate of 0.0001 and trained with the cross entropy loss function. The LSTM model is trained over 500 epochs with a batch size of 64 samples. The total number of trainable parameters of the LSTM model is 535,805.

Convolutional neural network (CNN) model. The CNN model in this paper has the following layers: an input layer (the input physiological signals are reshaped to 72000×1 voxels, each value normalized within the range [0, 1]), a convolutional layer (64 neurons), a flatten layer, a dropout layer (with a 0.2 dropout rate), a dense layer (with 32 neurons), a dropout layer (with a 0.2 dropout rate), and an output layer (with 5 neurons). ReLU is the activation function for the convolutional and dense layers, while softmax is the activation function for the output layer. The CNN model is optimized with rmsprop having a default learning rate of 0.0001 and trained with the cross entropy loss function. The CNN model is trained over 500 epochs with a batch size of 64 samples; the total number of trainable parameters is 147,456,453.

We further compare resource usage and performance across different models under k-fold cross-validation—namely, FDDLM, Vanilla DNN, LSTM, and CNN—by measuring the following metrics: execution time, trainable parameters, RAM usage (in GB) and accuracy. The evaluation results are shown in FIG. 9 and Table 3 (Of note, the values in Table 3 are the mean results under k-fold validation (k=5) and the values in FIG. 9 are the normalized results from Table 3). For FIG. 9 and Table 3, we observe that our FDDLM has the highest accuracy (i.e., 96.18%) with the lowest complexity (i.e., memory usage and execution time). These findings indicate that our model's predictions are more accurate while requiring a lower complexity than traditional machine learning models (i.e., Vanilla DNN, CNN, and LSTM).

Challenges and limitations of spirometry in COPD

Spirometry is a physiological test that measures the maximal air volume that an individual can inspire and expire with maximal effort, thus assessing the effect of a disease on lung function. Together with the medical history, symptoms, and other physical findings, it is an essential tool that provides essential information to clinicians in reaching a proper diagnosis. Indeed, standard spirometry is a laborious procedure: it needs preparation, a bronchodilation test, performance assurance, and evaluation.

Preparation: (1) The ambient temperature, barometric pressure, and time of day must be recorded. (2) Spirometers are required to meet International Organization for Standardization (ISO) 26782 standards, with a maximum acceptable accuracy error of 2.5%. (3) Spirometers need calibration daily, with calibration verification at low, medium, and high flow. (4) The technicians have to make sure that the device produces a hard copy of the expiratory curve plot to detect common technical errors. (5) The pulmonary function technician needs training in the optimal technique, quality performance, and maintenance. (6) There are activities that patients should avoid before testing, such as smoking or physical exercise. (7) Patients should be adequately instructed and then supported to provide a maximal effort in performing the test to avoid underestimating values and ultimately diagnosis errors.

Bronchodilation: (1) The forced expiratory volume in one second (FEV1) should be measured 10-15 minutes after the inhalation of 400 mcg short-acting beta2 agonist, or 30-45 minutes after 160 mcg short-acting anticholinergic, or the two combined. (2) Physicians also developed new withholding times for bronchodilators before bronchodilator responsiveness testing.

Performance assurance: (1) Spirometry should be performed using standard techniques. (2) The expiratory volume/time traces should be smooth and without irregularities, with α less than 1 second pause between inspiration and expiration. (3) The recording should be long enough to reach a volume plateau; it may take more than 15 seconds in severe cases. (4) Both forced vital capacity (FVC) and FEV1 should represent the biggest value obtained from any of three out of a maximum of eight technically good curves, and the values should vary by no more than 5% or 150 ml-whichever is bigger. (5) The FEV1/FVC ratio should be taken as the technically acceptable curve with the largest sum of FVC and FEV1.

Evaluation: (1) The measurements evaluation compares the results with appropriate reference values—specific to each age, height, sex, and race group. (2) The presence of a post-bronchodilator FEV1/FVC<0.70 confirms the presence of airflow limitation [74].

It is clear that the diagnosis process—primarily relying on spirometry—is pretty complex and, thus, prone to errors because of human intervention. The large university clinics, such as our Victor Babes clinic in Timisoara (and the other institutions included in our paper's recordings), avoid errors by carefully training their personnel and enforcing strict procedures. Additionally, experienced and well-trained physicians corroborate the spirometry results with other clinical data, such that diagnostic mistakes are highly improbable. However, we face a big problem with spirometry in primary care offices, which do not have all resources to includeently abide by the quality assurance steps (preparation, bronchodilation test, performance assurance, and evaluation). Hegewald ML et al. showed that most spirometers tested in primary care offices were not accurate, and the magnitude of the errors resulted in significant changes in the categorization of patients with COPD. Indeed, they obtained acceptable quality tests for only 60% of patients. In a similar study, the authors reported a spirometry accuracy varying from 69.1% to 81.4% in the primary care offices. These prior experimental studies and findings are significant for the medical community and constitute the motivation for our paper since primary care offices have an essential role in the early detection of COPD cases.

### Definition of COPD Stages

The diagnosis of COPD is based on persistent respiratory symptoms such as cough, sputum production, and dyspnea, together with airflow limitation (caused by significant exposure to smoking, noxious particles, or gases) evaluated with spirometry. The labels or disease stages are defined in the standard guide-line of the worldwide medical community [77]. Based on the FEV1 (forced expiratory volume in one second) value measured by spirometry, the Global Initiative for Chronic Obstructive Lung Disease (GOLD) guideline system categorizes airflow limitation into stages. In patients with FEV1/FVC (forced vital capacity)<0.70, the standard labels: (1) STAGE 1—mild: FEV1≥80%; (2) STAGE 2—moderate: 50%≤FEV1<80%; (3) STAGE 3—severe: 30%≤FEV1<50%; (4) STAGE 4-very severe: FEV1<30%. Additionally, in this paper, we assign the STAGE 0 label to patients without COPD (i.e., FEV1/FVC≥0.70).

### Early COPD Stages

Nowadays, there have been many debates in the literature regarding the early stage of COPD or the so-called asymptomatic COPD. Patients with COPD often underestimate the severity of the disease—primarily early morning and nighttime symptoms. The reasons may be the slow onset of their symptoms, cough due to a long cigarette smoking history, and dyspnea attributed to getting older. The majority of patients from a European cohort stated that they were not wholly frank with their doctors during visits when reporting their symptoms and quality of life.

Around 36% of patients who describe their symptoms as mild-to-moderate also admit to being too breathless to leave the house. For these reasons, there are two validated questionnaires (i.e., CAT and Modified Medical Research Council (mMRC)) that allow clinicians to accurately and objectively assess COPD symptoms. CAT is a globally used, 8 question, patient-filled questionnaire to evaluate the impact of COPD (cough, sputum, dyspnea, chest tightness) on health status. The range of CAT scores is 0-40. Higher scores denote a more severe impact of COPD on a patient's life. The mMRC Dyspnea Scale stratifies dyspnea severity in respiratory diseases, particularly COPD; it provides a baseline assessment of functional impairment attributable to dyspnea in respiratory diseases. Moreover, despite being highly symptomatic (mMRC≥2 and CAT≥10) and having at least one exacerbation, many COPD patients did not seek medical help, as they felt COPD symptoms as part of their daily smoking routine or due to aging. COPD awareness is poor among smokers; the smoker population underestimates their respiratory symptoms, while their exercise activity is reduced many times. Not surprisingly, 14.5% of the newly diagnosed COPD population was reported as asymptomatic in primary care clinics. Also, there is a high prevalence of COPD among smokers with no symptoms. We did not consider subjectively re-ported or observed clinical symptoms; instead, our analysis is based only on objectively measured parameters (i.e., physiological signals).

Spirometry as a screening tool for the early stage of the disease is not entirely robust. Indeed, spirometry can diagnose asymptomatic COPD, but its use is only recommended in smokers or individuals with a history of exposure to other noxious stimuli [83]. Despite having an apparent normal lung function, smokers with normal spirometry but a low diffusing capacity of the lung for carbon monoxide (DLCO) are at significant risk of developing COPD with obstruction to airflow—a category that may also be asymptomatic COPD. Moreover, no other disease markers are known to date to predict which patients with COPD of recent onset will progress to more significant disease severity.

Nonetheless, undiagnosed asymptomatic COPD has an increased risk of exacerbations and pneumonia. For these reasons, we need better initiatives for the early diagnosis and treatment of COPD [85]. Our method also aims at addressing the problem of early detection because it has an excellent accuracy at detecting early stages 1 and 2, which can also be detected with Spirometry. However, if our method can identify asymptomatic COPD that spirometry-based methods cannot see remains an open question; to that end, we need a longitudinal study starting with a significant cohort, which tracks the evolution of individuals over time to see if those predicted as asymptomatic COPD indeed develop the symptomatic form of the disease after several years.

FIG. 9 is a radar plot for measuring the complexity and prediction performance for the WestRo COPD dataset across different deep learning models under k-fold validation (k=5), including a fractional-dynamics deep learning model (FDDLM), Vanilla deep neural network (DNN), long short-term memory (LSTM), and convolutional neural network (CNN). We normalized all the values represented in this plot.

Table 3 below shows complexity and prediction performance for the WestRo COPD dataset across different deep learning models under k-fold validation (k=5), including a fractional dynamics deep learning model (FDDLM), Vanilla deep neural network (DNN), long short-term memory (LSTM), and convolutional neural network (CNN). ⬆/⬇ indicates higher/lower values are better. All results are evaluated on the same machine for fair comparison.

| | FDDLM | DNN | LSTM | CNN |
|---|---|---|---|---|
| RAM page (GB) ↓ | 0.98 | 8.17 | 7.68 | 9.35 |
| Execution time (sec) ↓ | 1076 | 47,590 | 205,135 | 345,085 |
| Trainable parameters ↓ | 74,105 | 21,630,905 | 535,805 | 147,456,453 |
| Test accuracy (%) ↑ | 98.66 | 77.72 | 78.54 | 36.12 |
| Loss ↓ | 0.2170 | 0.9601 | 0.5728 | 0.6245 |

COPD is one of the leading causes of death worldwide, usually associated with smoking and environmental occupational exposures. Prior studies have shown that current COPD diagnosis (i.e., spirometry test) can be unreliable because the test can be difficult to do and depends on an adequate effort from the testee and supervision of the testor. Moreover, the extensive early detection and diagnosis of COPD is challenging.

We address the COPD detection problem by constructing two novel COPD physiological signals datasets (e.g., 4432 medical records from 54 patients in the WestRo COPD dataset and 13824 medical records from 534 patients in the WestRo Porti COPD dataset), demonstrating their complex coupled fractal dynamical characteristics, and performing a rigorous fractional-order dynamics deep learning analysis to diagnose COPD with high accuracy. We find that the fractional-order dynamical modeling can extract distinguishing signatures from the physiological signals across patients with all COPD stages—from stage 0 (healthy) to stage 4 (very severe). We exploit these fractional signatures to develop and train a deep neural network that predicts the suspected patients' COPD stages based on the input features (such as thorax breathing effort, respiratory rate, or oxygen saturation levels). We show that our COPD diagnostics method (fractional dynamic deep learning model) achieves a high prediction accuracy (98.66%±0.45%) on WestRo COPD dataset and can serve as an excellent and robust alternative to traditional spirometry-based medical diagnosis. Our FDDLM for COPD diagnosis also presents high prediction accuracy when validated by a dataset with different physiological signals recorded (i.e., 94.01%+0.61% for predicting the COPD stages in the WestRo COPD dataset with the model trained on the WestRo Porti COPD dataset, and 90.13%±0.89% for predicting in the WestRo Porti COPD with the model trained on WestRo COPD).

### Sensitivity, Specificity, and Precision Rate of the Confusion Matrices

The WestRo COPD dataset includes of physiological signals recorded over consecutive patients from four Pulmonology Clinics in Western Romania (Victor Babe Hospital—VB, Medicover 1—MID1, Medicover 2—MD2, and Cardio Prevent—CP clinics). This supplementary material displays detailed results about the confusion matrices presented in the manuscript (FIG. 7). We generated the confusion matrices with our fractional dynamics deep learning model (FDDLM), the vanilla DNN model, and the LSTM model and by holding out data gathered at each institution—at a time—as test sets. (The vanilla DNN and LSTM models have a similar network structure with the fractional dynamics deep learning model, except the input size.) We analyze the sensitivity, specificity, and precision of these confusion matrices to measure the performance of each machine learning model. We present the results in Tables S1, S2, and S3. The results in these tables show that, despite LSTM and vanilla DNN models having high specificity, the prediction sensitivity and precision of these two models are not good (with the worst sensitivity values dropping to 36.77% and 35.33%). In contrast, our fractional dynamics deep learning model shows relatively high values across the three performance measurements. We conclude that our model predicts patients' COPD stage with higher accuracy than vanilla DNN and LSTM models—without overfitting—therefore representing an effective alternative to the spirometry-based diagnostic.

Table S1 below shows the COPD stage prediction results with the fractional dynamics deep learning model by holding out data gathered from each institution at a time as the test set.

| Medical | | | | | | |
|---|---|---|---|---|---|---|
| institution | Parameters | Stage 0 | Stage 1 | Stage 2 | Stage 3 | Stage 4 |
| VB | Sensitivity | 97.96% | 86.45% | 99.99% | 99.62% | 99.99% |
| | specificity | 99.99% | 99.99% | 97.01% | 99.88% | 99.79% |
| | Precision | 99.99% | 99.99% | 94.42% | 99.62% | 98.84% |
| MD1 | Sensitivity | 99.99% | 98.76% | 94.77% | 97.12% | 95.86% |
| | specificity | 98.85% | 98.10% | 99.71% | 99.33% | 99.99% |
| | Precision | 93.29% | 89.89% | 99.52% | 97.12% | 99.99% |
| MD2 | Sensitivity | 98.73% | 98.20% | 86.31% | 94.51% | 99.34% |
| | specificity | 96.70% | 96.00% | 98.70% | 99.77% | 99.78% |
| | Precision | 83.87% | 82.00% | 99.44% | 98.85% | 98.68% |
| CP | Sensitivity | 95.60% | 95.83% | 99.14% | 99.35% | 99.52% |
| | specificity | 99.99% | 99.66% | 99.10% | 99.23% | 99.74% |
| | Precision | 99.99% | 97.87% | 98.29% | 97.47% | 98.56% |

### Convolutional Neural Network (CNN) Training and Testing Results

FIGS. 10A, 10B, 10C, 10D, 10D, 10E, 10F illustrate training and testing results in terms of accuracy, loss, and AUC of the convolutional neural network model for the k-fold cross-validation and hold out validation, including training/testing accuracy (FIG. 10A), loss (FIG. 10B), and AUC (FIG. 10C) for convolutional neural network model under k-fold cross-validation, where the training processes utilize physiological signals, and training/testing accuracy (FIG. 10D), loss (FIG. 10E), and AUC (FIG. 10F) for convolutional neural network model under hold-out validation, where the training processes utilize physiological signals.

FIGS. 10A, 10B, and 10C illustrate the training and testing results in terms of accuracy, loss, and AUC for the CNN model trained with physiological signals (raw data) under k-fold cross-validation (k=5). The testing accuracy of CNN indicates a lower predicting accuracy (36.12%±0.001%) compared with our fractional dynamics model (98.66%±0.447%). In FIGS. 10D, 10E, 10F present the training and testing results in terms of accuracy, loss, and AUC for the CNN model trained with physiological signals (raw data) under hold-out validation. The testing accuracy under hold-out validations is 35.51% 0.009%, significantly lower than our model's predicting accuracy (95.88%1.76%). Hence, we conclude that our fractional-dynamics deep learning model predicts patients' COPD stages with higher accuracy than the vanilla DNN, LSTM, and CNN models trained with physiological signals.

Table S2 below shows the COPD stage prediction results with the vanilla DNN model by holding out data gathered from each institution at a time as the test set.

| Medical | | | | | | |
|---|---|---|---|---|---|---|
| institution | Parameters | Stage 0 | Stage 1 | Stage 2 | Stage 3 | Stage 4 |
| VB | Sensitivity | 91.16% | 36.77% | 94.09% | 89.02% | 74.71% |
| | specificity | 94.90% | 96.33% | 93.34% | 94.91% | 96.91% |
| | Precision | 73.22% | 61.96% | 87.72% | 84.53% | 81.41% |
| MD1 | Sensitivity | 79.61% | 54.04% | 86.62% | 72.60% | 77.24% |
| | specificity | 96.75% | 90.48% | 93.54% | 96.32% | 94.07% |
| | Precision | 79.60% | 49.15% | 89.88% | 82.07% | 66.27% |
| MD2 | Sensitivity | 97.25% | 51.04% | 92.67% | 94.52% | 81.64% |
| | specificity | 96.59% | 96.64% | 93.93% | 96.36% | 98.52% |
| | Precision | 81.57% | 71.53% | 88.84% | 88.51% | 68.93% |
| CP | Sensitivity | 78.48% | 52.09% | 88.51% | 97.80% | 79.61% |
| | specificity | 96.81% | 91.01% | 92.11% | 96.61% | 99.56% |
| | Precision | 81.04% | 51.78% | 87.44% | 85.57% | 96.80% |

Table S3 below shows the COPD stage prediction results with the LSTM model by holding out data gathered from each institution at a time as the test set.

| Medical | | | | | | |
|---|---|---|---|---|---|---|
| institution | Parameters | Stage 0 | Stage 1 | Stage 2 | Stage 3 | Stage 4 |
| VB | Sensitivity | 99.99% | 50.63% | 80.91% | 87.12% | 88.82% |
| | specificity | 91.90% | 92.55% | 96.89% | 99.99% | 96.81% |
| | Precision | 65.33% | 52.98% | 92.90% | 99.99% | 83.43% |
| MD1 | Sensitivity | 75.16% | 73.29% | 78.91% | 73.07% | 82.76% |
| | specificity | 96.23% | 86.16% | 98.95% | 97.22% | 94.18% |
| | Precision | 76.16% | 47.39% | 98.02% | 85.87% | 68.18% |
| MD2 | Sensitivity | 66.46% | 35.33% | 97.07% | 70.72% | 93.42% |
| | specificity | 98.42% | 93.19% | 86.26% | 98.98% | 93.31% |
| | Precision | 87.50% | 47.97% | 80.86% | 94.57% | 68.93% |
| CP | Sensitivity | 99.99% | 53.13% | 77.80% | 89.35% | 99.03% |
| | specificity | 92.32% | 91.14% | 99.99% | 99.80% | 97.13% |
| | Precision | 66.91% | 49.76% | 99.99% | 99.28% | 86.13% |

### Hurst Exponents of Physiological Signals

In this section, we show the Hurst exponents in non-derived signals (Thorax, Oxygen Saturation, Pulse, Plethysmograph, Nasal Pressure, and Abdomen) among COPD patients for the intermediate-stage patients (q∈[−5, 5]). The Hurst exponent measures the long-term memory of time series; different Hurst exponent values reveal different evolving variations in time series with different fractal features. FIGS. 11A, 11B, 11C, 11D, 11E, 11F, 11G, 11H, 11I present the H(q) confidence intervals for Abdomen, Thorax, and Pulse signals, for different q values, across different COPD stages (i.e., 0, 1, 2, 3) with 95% confidence intervals. FIGS. 11A, 11B, 11C, 11D, 11E, 11F, 11G, 11H, 11I illustrate multifractal analysis of 3 non-derived physiological signals (Abdomen, Thorax, and Pulse) extracted from non-COPD individuals (i.e., stage 0) and stage 4 COPD patients with 95% confidence interval. We present the generalized Hurst exponent (H(q)) as a function of q-th order moments (q∈[−5, 5]) for Abdomen (a), Thorax (d), and Pulse (g) signals extracted from stage 1 patients; H(q) as a function of q-th order moments (q∈[−5, 5]) for Abdomen (b), Thorax (e), and Pulse (h) signals extracted from stage 2 patients; H(q) as a function of q-th order moments (q∈[−5, 5]) for Abdomen (c), Thorax (f), and Pulse (i) signals extracted from stage 3 patients. For reference, the H(q) function for all physiological signals (Abdomen, Thorax, and Pulse) extracted from the healthy people (stage 0) are plotted in (a-i) panels as purple curves.

FIGS. 12A, 12B, 12C, 12D, 12E, 12F, 12G, 12H, 12I presents the H(q) confidence intervals for Nasal Pressure, Oxygen Saturation, and Plethysmograph signals, for different q values, across different stages (0, 1, 2, 3) with 95% confidence intervals. FIGS. 12A, 12B, 12C, 12D, 12E, 12F, 12G, 12H, 12I illustrate multifractal analysis of 3 non-derived physiological signals (Nasal Pressure, Oxygen Saturation, and Plethysmograph) extracted from non-COPD individuals (i.e., stage 0) and stage 4 COPD patients with 95% confidence interval. We present the generalized Hurst exponent (H(q)) as a function of q-th order moments (q∈[−5, 5]) for Nasal Pressure (a), Oxygen Saturation (d), and Plethysmograph (g) signals extracted from stage 1 patients; H(q) as a function of q-th order moments (q∈[−5, 5]) for Nasal Pressure (b), Oxygen Saturation (e), and Plethysmograph (h) signals extracted from stage 2 patients; H(q) as a function of q-th order moments (q∈[−5, 5]) for Nasal Pressure (c), Oxygen Saturation (f), and Plethysmograph (i) signals extracted from stage 3 patients. For reference, the H(q) function for all physiological signals (Nasal Pressure, Oxygen Saturation, and Plethysmograph) extracted from the healthy people (stage 0) are plotted in the (a-i) panels as purple curves.

In both FIGS. 11A-11I and FIG. 12A-12I, the H(q) of physiological signals extracted from healthy people (stage 0) are plotted as purple patterns in each panel for reference. From these two figures, we notice that different stages have different H(q) confidence intervals under the same q values, which is the evidence that all physiological signals extracted from patients with different COPD stages have different fractional dynamic characteristics.

For reference, FIGS. 13A, 13B, 13C, 13D, 13E, 13F, 13G, 13H, 13I and FIGS. 14A, 14B, 14C, 14D, 14E, 14F, 14G, 14H, 14I present the comparison of terms of H(q) intervals for COPD patients for different q values (q∈{−5, 0, 5}. FIGS. 13A, 13B, 13C, 13D, 13E, 13F, 13G, 13H, 13I illustrate comparison in multifractal features between 3 non-derived physiological signals extracted from patients with different COPD stages. We plot the generalized Hurst exponent (H(q)) for Abdomen signals extracted from COPD patients in different stages under different q-th order moments (q=−5 (a), q=0 (b), and q=5 (c)); Generalized Hurst exponent (H(q)) for Thorax signals extracted from COPD patients with different stages under different q-th order moments (q=−5 (d), q=0 (e), and q=5 (f)); H(q) for Pulse signals extracted from COPD patients in different stages under different q-th order moments (q=−5 (g), q=0 (h), and q=5 (i)). FIG. 14A, 14B, 14C, 14D, 14E, 14F, 14G, 14H, 14I illustrate Comparison of multifractal features between 3 non-derived physiological signals extracted from patients with different COPD stages. We plot the generalized Hurst exponent (H(q)) for Nasal Pressure signals extracted from COPD patients in different stages under different q-th order moments (q=−5 (a), q=0 (b), and q=5 (c)); H(q) for Oxygen Saturation signals extracted from COPD patients with different stages under different q-th order moments (q=−5 (d), q=0 (e), and q=5 (f)); H(q) for Plethysmograph signals extracted from COPD patients in different stages under different q-th order moments (q=−5 (g), q=0 (h), and q=5 (i)). Hence, it makes sense to analyze the spatial coupling between these physiological processes (signals) across time to investigate the different fractional features across signals recorded from different COPD patients.

FIGS. 15A, 15B, 15C, 15D, 15E, 15F, 15G, 15H, 15I, 15J, 15K, 15L present the comparison of terms of H(q) intervals for COPD patients for different q values (q∈{−5, 0, 5} to show the scaling functions of physiological signals extracted from all stage 4 patients and the healthy people (stage 0) in our dataset (where q∈[−5, 5]). The purple areas are the confidence intervals. FIGS. 15A, 15B, 15C, 15D, 15E, 15F, 15G, 15H, 15I, 15J, 15K, 15L illustrate geometry of fluctuation profiles for the COPD-relevant physiological signals recorded from healthy people and stage 4 patients. We compute the scaling function from non-derived physiological signals (Abdomen, Thorax, Oxygen Saturation, Plethysmograph, Nasal Pressure, and Pulse) with the exponents q∈[−5, 5]. FIGS. 15A, 15B, 15C, 15G, 15H, 15I correspond to all healthy people in our datasets (stage 0), while FIGS. 15D, 15E, 15F, 15J, 15K, 15L correspond to all the stage 4 COPD patients in our datasets. We also used the MF-DFA toolbox provided by to re-compute the multifractal scaling functions of the signals collected from stage 0 and 4 participants (we choose the same participants in FIGS. 2A-2L for reanalyzing the S(q)).

The results are shown in FIG. 16A-16L. FIGS. 16A-16L illustrate the geometry of fluctuation profiles for the COPD-relevant physiological signals recorded from a normal abdomen and a stage 4 COPD abdomen. We use the toolbox provided by 1 to calculate the scaling functions from 6 raw physiological signals: Abdomen, Thorax, Oxygen Saturation, Plethysmograph, Nasal Pressure, and Pulse, where the exponents are q∈[−5, 5]. FIGS. 16A, 16B, 16C, 16G, 16H, 16I show signals recorded from a healthy person, FIGS. 16D, 16E, 16F, 16J, 16K, 16L show signals recorded from a representative stage 4 COPD patient.

### Standard Deviation Analysis and MSE Analysis for Scaling Functions

To show the fractal difference between raw signals recorded from stage 0 and 4 participants in detail, we generated FIGS. 18A and 18B to show the correlations between scale (s) vs. standard deviations (STDs) of S_{F}(q) and q values vs. STDs of S_{F}(s). FIGS. 18A and 18B illustrate scale (s) vs. standard deviation (STD) of S_{F}(q) between stage 0 (FIG. 18A) and stage 4 (FIG. 18B) participants. FIGS. 18A and 18B respectively show the s vs. STDs of S_{F}(q) for stage 0 and 4 participants across 6 physiological signals (abdomen, nasal pressure, oxygen saturation, plethysmograph, pulse, and thorax). When s=L, in stage 0, the STD for all six signals is close to zero; in stage 4, the STDs for all signals are larger than 0.5 (except nasal pressure). In some examples, the multifractal signals have smaller STDs of log(S(q, L)) than the multifractal noise (monofractal). Accordingly, we can claim that the raw signals across Stage 0 and Stage 4 participants have different fractal features. To measure the error in terms of scale and fluctuation, we show the mean square error (MSE) of the regressed thin pink lines in FIGS. 11A-11I.

We show the results in FIGS. 19A and 19B, where (FIG. 19A) and (FIG. 19B) show the MSE for stage 0 and 4 participants, respectively. FIGS. 19A and 19B illustrate Mean squared error (MSE) for the regressed lines in FIGS. 11A-11I for stage 0 and stage 4 participants.

### Error Analysis and Learning Process Visualization

Although the fractional dynamics deep learning model FDDLM provides relatively high prediction accuracy under k-fold cross-validation and hold-out validation, the detection accuracy drops a bit in the hold-out validation (from 98.66% to 95.88%). The reason is that the data recorded from each medical institution are unbalanced, as our cohort is a real-life population. The Victor Babes (VB) and Cardio Prevent (CP) are two large clinics with COPD patients who are more willing to get the diagnosis or medical treatment in large units or hospitals than in small clinics-especially in the case of severe and very severe COPD stages. Thus, signals recorded from VB and CP are more comprehensive/diverse than from Medicover 1 (MD1) and Medicover 2 (MD2). In the hold-out section, we balanced the data across different institutions using over-sampling and under-sampling approaches. Indeed, the unbalanced data collection is the leading cause of the prediction accuracy drop in the hold-out section.

FIG. 17A illustrates data distribution across the medical institutions. FIG. 17B illustrates percentage of COPD stages for each medical institution. In FIGS. 17A and 17B, we present the data distribution across the medical institutions; in the left-hand side panel, we have the distribution of the absolute number of individuals, while in the right-hand side panel, we present the percentage of COPD stages for each institution. Both panels emphasize the data unbalance problem-MD1 and MD2 have a small number of individuals, and only CP has individuals representing all COPD stages. To handle these imbalanced data across different institutions, we perform over-sampling and under-sampling techniques to guarantee that all institutions have a similar number of testing samples across different COPD stages (e.g., since MD2 does not have stage 4 samples, we move two stage-4 patients from VB to MD2 and over-sample/under-sample the data to ensure the data balance).

To provide further insight, we present the learning process in the hidden layers of our fractional dynamics deep learning model for k-fold and hold-out validation by employing the t-Distributed Stochastic Neighbor Embedding (t-SNE) visualization algorithm. Besides the input-, dropout-, and output-layers, we have two hidden dense layers in our deep learning model (the first layer has 300 neurons and the second layer has 100 neurons). The t-SNE technique is an approach to reduce data dimensionality in two or three-dimension maps. In this work, we regard the outputs of the first dense layer as a 300-dimension coordinate and the second dense layer as a 100-dimension coordinate. Then, we employ the t-SNE technology to reduce these coordinates to two-dimension coordinates and visualize the learning processes in these two hidden dense layers.

FIGS. 20A, 20B, 20C, 20D, 20E, 20F, 20G, 20H, 20I illustrate representation of the learning process about hold-out and k-fold cross-validation in the first hidden layer. We present the t-SNE visualization of the first hidden layer in the fractional dynamics deep learning model of the COPD dataset under hold-out (a-d) and k-fold (e-i) cross-validation. In each scatterplot, the colors of the points indicate the classes of the corresponding COPD stages, and the shape of the points represents the fractional dynamics deep learning model's prediction results (i.e., the round shape indicates a correct prediction, and the ‘x’ mark indicates an error). FIG. 20 presents the learning processes visualization of the first hidden layer. FIGS. 20A, 20B, 20C, 20D show the t-SNE results for hold-out validation. FIGS. 20E, 20F, 20G, 20H, 20I show the t-SNE results for k-fold cross-validation. The color of scatters presents the label of each individual case, and each point's shape indicates the prediction results of our fractional dynamics deep learning model (where the round markers indicate the correct prediction results and the ‘x’ markers indicate errors). FIGS. 20A, 20B, 20C, 20D hold out the data recorded from different institutions as test sets (VB (a), MD1 (b), MD2 (c), and CP (d)), and (e-i) hold out the 0-20% (e), 20%-40% (f), 40%-60% (g), 60%-80% (h), 80%-100% (i) data as test sets, respectively. In FIGS. 20A, 20B, 20C, 20D, 20E, 20F, 20G, 20H, 20I, we can find that scatters under the same category tend to build clusters, and some clusters overlap. The overlapping patterns are difficult to learn for the deep learning model (errors mostly occur in these overlapping ranges), and the isolated clusters with clear boundaries are the easiest part to recognize for the deep learning model. By comparing the t-SNE figures across hold-out validation (FIGS. 20A, 20B, 20C, 20D) and k-fold cross-validation (FIGS. 20E, 20F, 20G, 20H, 20I), we find that the scatters in hold-out validation have more extensive overlapping ranges than the scatters in k-fold cross-validation. This observation illustrates that the datasets used in hold-out validation are harder to distinguish for the deep learning model than the dataset used in k-fold cross-validation.

FIGS. 21A, 21B, 21C, 21D, 21E, 21F, 21G, 21H, 21I illustrate representation of the learning process about hold-out and k-fold cross-validation in the last hidden layer. We present the t-SNE visualization of the last hidden layer in the fractional dynamics deep learning model of the COPD dataset under hold-out (FIGS. 21A, 21B, 21C, 21D) and k-fold (FIGS. 21E, 21F, 21G, 21H, 21I) cross-validation. In each scatterplot, the colors of the points indicate the classes of the corresponding COPD stages, and the shape of the points represents the fractional dynamics deep learning model's prediction results (i.e., the round shape indicates a correct prediction, and the ‘x’ mark indicates an error). FIGS. 21A, 21B, 21C, 21D, 21E, 21F, 21G, 21H, 21I present the learning processes visualization of the last hidden layer in our fractional dynamics deep learning model. FIGS. 21A, 21B, 21C, 21D hold out the data gathered from different institutions as test sets (VB (FIG. 21A), MD1 (FIG. 21B), MD2 (FIG. 21C), and CP (FIG. 21D)), and (FIGS. 21E, 21F, 21G, 21H, 21I) hold out the 0-20% (FIG. 21E), 20%-40% (FIG. 21F), 40%-60% (FIG. 21G), 60%-80% (FIG. 21H), 80%-100% (FIG. 21I) data as test sets, respectively. As one can observe, FIGS. 21A-21I have the same occurrence as in FIGS. 20A-20I; namely, that the datasets used in hold-out validation are more difficult to learn than the dataset used in k-fold cross-validation. Moreover, by comparing FIGS. 20A-20I and FIGS. 21A-21I, we observe that overlapping patterns in each FIGS. 21A-21I panel are narrower than the corresponding panel FIGS. 20A-20I. This provides numerical evidence that our fractional dynamics deep learning model FDDLM provides a feasible learning process and gives desired prediction results.

### Linear Classifier of Coupling Matrices

Besides the deep learning architecture in our fractional dynamics deep learning model FDDLM, we also utilize linear classifier models to mine the complexity of coupling matrices A. Logistic regression is traditionally a linear classifier; it is one of the most used two-class (and multi-class) classification machine learning algorithms. When we use logistic regression instead of deep learning on the A matrices, the accuracy of COPD prediction is 94.61%±0.98% with k-fold and 86.68%±0.82% with hold-out validation. The COPD prediction accuracy for logistic regression is lower with the k-fold validation and significantly lower with the hold-out validation than the fractional dynamics deep learning model (i.e., 98.66%±0.45% and 95.88%±1.76%, respectively). We show the confusion matrices for logistic regression in FIGS. 22A, 22B, 22C, 22D, 22E, 22F, 22G, 22H, 22I.

FIGS. 22A, 22B, 22C, 22D, 22E, 22F, 22G, 22H, 22I illustrate the confusion matrices resulted from the logistic regression model. The k-fold cross validation (k=5). In each iteration, we hold out 0-20% (FIG. 22A), 20%-40% (FIG. 22B), 40%-60% (FIG. 22C), 60%-80% (FIG. 22D), and 80%-100% data (FIG. 22E) as test sets. For the hold-out validation, we built the test sets by holding out data recorded from one institution at a time: VB (FIG. 22F), MD1 (FIG. 22G), MD2 (FIG. 22H), and CP (FIG. 22A). FIGS. 22A, 22B, 22C, 22D, 22E are the prediction results on test sets for the k-fold validation, and FIGS. 22F, 22G, 22H, 22I show the prediction results for the hold-out validation. Of note, for the linear classifier model, the accuracy for the k-fold validation is significantly higher than the accuracy for hold-out validation. The explanation is that our data is unbalanced in different medical institutions (for detailed information, see the Supplementary material's section Error analysis and learning process visualization).

Besides the logistic regression, we also investigate another classifier model as a reference, namely, SVM. Support-vector machines (SVM) are a particular case of linear classifiers based on the margin maximization principle. When using SVMs instead of deep learning on the A matrices, the accuracy of COPD prediction becomes 94.74%±0.24% with k-fold and 92.62%±0.39% with hold-out validation. We show the confusion matrices for SVM under k-fold and hold-out validations in FIGS. 23A, 23B, 23C, 23D, 23E, 23F, 23G, 23H, 23I.

FIGS. 23A, 23B, 23C, 23D, 23E, 23F, 23G, 23H, 23I illustrate the confusion matrices resulted for the SVM. For the k-fold cross validation (k=5). In each iteration, we hold out 0-20% (FIG. 14A), 20%-40% (FIG. 14B), 40%-60% (FIG. 14C), 60%-80% (FIG. 14D), and 80%-100% data (FIG. 14E) as test sets. For the hold-out validation, we built the test sets by holding out data recorded from one institution at a time: VB (FIG. 14F), MD1 (FIG. 14G), MD2 (FIG. 14H), and CP (FIG. 14I). In FIGS. 23A, 23B, 23C, 23D, 23E, 23F, 23G, 23H, 23I, FIGS. 23A, 23B, 23C, 23D, 23E are the prediction results on test sets for the k-fold validation, and FIGS. 23F, 23G, 23H, 23I show the prediction results for the hold-out validation. Like the logistic regression model, the k-fold validation accuracy is significantly higher than the accuracy for hold-out validation in the SVM model. The reason is that our data is unbalanced across the institutions. We conclude that our fractional dynamics deep learning model outperforms these two linear classifier models (i.e., logistic regression and SVM) in terms of prediction accuracy under k-fold and hold-out validation. Indeed, learning from many matrices A is a complicated process that requires deep learning.

### Standard Questionnaires, Exacerbation History, and Comorbidities of COPD Patients

In this section, we provide the data description about the standard questionnaires (CAT and MRC), exacerbation history, and comorbidities about all the COPD patients in our datasets. Questionnaires are recommended for the management of COPD. Since 2011, the GOLD guidelines have included the following questionnaires in the assessment of COPD patients: the modified Medical Research Council (mMRC) dyspnea scale, the COPD assessment test (CAT), and the clinical COPD questionnaire (CCQ); they are carefully-designed high-quality questionnaires, but information on the feasibility for routine use is scarce. Nonetheless, questionnaires are both quick to complete and have good acceptability by the patient. In addition, the agreement between electronic and paper versions of the questionnaires was high. FIG. 24 presents the CAT and MRC scores for COPD patients across different stages in our paper's WestRo cohort. FIG. 24A illustrates CAT scores for COPD patients across different COPD stages. FIG. 24B illustrates MRC scores for COPD patients across different COPD stages.

However, we made sure that our diagnoses—that lead to classifying patients in COPD stages 0 to 4—are reliable by reviewing each case after several months (including of a complete medical check-up, including spirometry).

We also collected data about the history of exacerbation for all the patients. When they were evaluated, their condition was stable. The data about the exacerbation history in the previous year for our patients is 16 patients without exacerbations, 25 patients with one exacerbation, 5 patients with two exacerbations, and 1 with three exacerbations. We considered it essential to make all the measurements and evaluations in a stable COPD phase of the disease. Therefore, we performed all functional tests when patients were not experiencing an acute exacerbation. The reason is that there is an increase in hyperinflation and gas trapping during an exacerbation, with a reduced expiratory flow and increased dyspnea.

Exacerbations are important in the management of COPD patients. There is an frequent exacerbator phenotype with an increased risk of hospitalization and death. Exacerbations of COPD have a considerable impact on patients' health status and exercise capacity and have a cumulative effect on lung function7. However, longitudinal changes in FEV1 are not significantly associated with the exacerbation risk. Exacerbations can be found in any COPD stage8. In addition, a single COPD exacerbation may also result in a significant increase in lung function decline rate9. To investigate the impact of exacerbations, we pick 4 patients from each COPD stage (i.e., stage 2, 3, and 4) and hold out untrained signal samples as test sets (where 2 of them have 2 or more exacerbations and the other 2 have less than 2 exacerbations). We apply our model to make predictions about these test sets to reveal whether the exacerbation history will influence our prediction accuracy (the reason for choosing 4 patients in each stage from our datasets is that we want to maintain the test sets balanced during the prediction process). In stage 2, the prediction accuracy of samples gathered from patients with less than 2 exacerbations (category 1) is 99.01% and the prediction accuracy of samples generated from patients with more than 2 exacerbations (category 2) is 98.07%. In stage 3, the prediction accuracy is 99.28% for category 1 and 98.23% for category 2. In stage 4, the prediction accuracy of category 1 is 97.88%, and of category 2 is 98.22%. Hence, we conclude that the distribution of exacerbations history and comorbidities across patients and stages correlated with the high prediction rate of our method suggests that our fractal dynamics deep learning model FDDLM is not influenced by comorbidities or exacerbation history. (We have patients with exacerbation history in all COPD stages, except—of course—stage 0 COPD; we also have many COPD stages represented in each comorbidity, please see FIGS. 25A and 25B.)

FIG. 25A illustrates exacerbations across patients under different COPD stages. FIG. 25B illustrates a percentage of COPD stages for each comorbidity (e.g., Cardiovascular comorbidities (CC), Cancers (CA), Metabolic comorbidities (MC), Psychiatric comorbidities (PC), Renal disease (RD)).

Comorbidities in COPD are expected at any stage of the disease. The most common comorbidities accompanying COPD include cardiovascular diseases, metabolic disorders, osteoporosis, musculo-skeletal dysfunction, anxiety/depression, cognitive impairment, gastrointestinal diseases, and respiratory conditions such as asthma, bronchiectasis, pulmonary fibrosis, and lung cancer. Comorbidities are known to pose a challenge in the assessment and effective management of COPD. However, the mechanistic links between COPD and its comorbidities are still not fully understood. The variability of the clinical presentation in COPD interacts with comorbidities to form a complex clinical scenario for clinicians to deal with¹². As a result, attention needs to be paid to assessing and managing comorbidities in COPD in both clinical and research settings. In addition, for the effective management of comorbidities in COPD, there is a need for reliable measurement tools that can assist in improving clinical outcomes. In this work, we record the following comorbidities: Cardiovascular comorbidities (CC); Cancers (CA); Metabolic comorbidities (MC); Psychiatric comorbidities (PC); and Renal disease (RD). The results are shown in FIG. 25B.

### Convergence of Coupling Matrix

To evaluate our fractional dynamics deep learning model's efficiency, we investigate the sufficient length of the signals to make stable conclusive results. FIG. 26 displays the convergence of matrix A across different stages of patients. The Wasserstein distance is a metric for the differences between two distributions (the small distance value implies that two distributions are similar.) FIG. 26 illustrates the Wasserstein distance between the coupling matrices A over time. In FIG. 26, we calculate the coupling matrices for different patients with different COPD stages by the length of the physiological signals (time-series) and calculate the Wasserstein distances between two neighboring time stamps (e.g., we calculate the Wasserstein distance between two coupling matrices generated from time-sequence 0 to t seconds and 0 to t+1 seconds). From FIG. 26, we observed that—for each COPD stage—the Wasserstein distance of coupling matrices converges to a small value (less than 0.02) after 600 seconds. This observation suggests that after 10 minutes, the coupling matrices' elements remain constant. Thus, a 10 minutes record interval is sufficient for our model to make the predictions.

### Early Viral Infection Detection

To emphasize the generality and correctness of our fractional dynamics method, we also analyze the Biochronicity viral prediction dataset with our model. The Biochronicity pilot study—relevant to the current work—is summarized as follows. The human rhinovirus (HRV)39 was injected into 18 human subjects monitored from 4 days before to 4 days after injection.

Out of the 18 patients, 11 were shedding, showed symptoms after 3-4 days, and were marked as infected; the remaining 7 were considered healthy. The E4 Empatica physiological sensor records the temperature, blood pressure-volume (heart rate), accelerometer (3-axis), skin temperature, and electrodermal activity. The goal was to detect the viral infection in less than 24 hrs from the inoculation point. The dataset imposes several practical challenges: first, paucity of data as there are only 18 samples; second, the detection has to be made as early as 24 hrs after the viral inoculation—when the symptoms are not prominent enough to be detected by a medical expert.

Next, we explain the fractional dynamics-based method/pipeline in viral prediction. We take the 3 physiological time-series features, electrodermal activity (EDA), body temperature (TEMP), and inter-beat interval (IBI). For a given inoculation point, the 3-dimensional time series are broken into pre- and post-viral infection data. For each pre- and post-viral data, a sliding window mechanism with a window length of 3000 samples and a sliding length of 100 samples (the choice is made by cross-validation over the window length and sliding length grid) is fitted using a fractional dynamical model with spatial coupling, from which we obtain the fractional coefficients α. Each window slide results in three fractional coefficients (one for each physiological feature), and then we estimate the probability density of a for pre- and post-viral periods. Finally, we use the Kullback-Leibler (KL) divergence between the pre- and post-viral fractional distributions as the feature for differentiating the infected and healthy subjects. The intuition is that the fractional coefficient captures the scaling behavior of the time series; by computing the difference between the distributions, we assume that healthy and infected subjects have different scaling behavior in their physiological activities.

One of the crucial assumptions in early viral detection is the knowledge of the viral inoculation point. From a practical standpoint, it is not possible to obtain such information. Therefore, we evaluate the efficacy of our model by moving the assumed viral inoculation point from the actual infection time in both directions. FIG. 27 shows the early viral prediction using fractional dynamics. The Type-I and Type-II errors are shown by shifting the model's viral injection point from the actual reference (shift=0). FIG. 27 illustrates the plot of classification performance in terms of Type-I and Type-II errors when the inoculation point is shifted in positive and negative increments. Due to the scarcity of data, we use a leave-one-out-based resampling for the classification task. The ‘V’-like structure of the Type-I error is informative because it shows the best performance (as few as 3 errors) close to the actual inoculation point (0 hrs) while the loss in performance on moving in either direction from 0 hours. The error increase (in either direction) is caused by shifting the inoculation point; the past and future of the patient become more or less the same. Hence, the classifier would falsely label the patient as healthy or, in other words, make a Type-I error. Another important observation is that the best performance—a total of 3 errors out of 18 subjects (3 Type—I and 0 Type-II)—is achieved not at the actual infection time (shift=0 hours) but when the infection time is assumed delayed by +1 hour. A possible explanation is that the physiological effects take time to trigger; therefore, a delayed version of the separating boundary produces better differentiation between the pre- and post-viral data. Finally, another important outcome of the fractional model is that the Type-II error is roughly constant and is consistently low, which is required behavior.

### WestRo Porti COPD Dataset Analysis

To test the generalization capabilities of our framework, we use the WestRo Porti COPD dataset including of 13824 physiological signals samples, recorded from 534 patients (232 COPD patients and 302 non-COPD patients) in the Victor Babes hospital.

The dataset recorded 6 physiological signals from each patients (for detailed information about WestRo Porti COPD dataset, see the Method section Data collection). This section evaluates the accuracy, loss, and area under the curve (AUC). The results generated from different models (fractional-dynamics deep learning model, Vanilla DNN, LSTM, and CNN) are validated using the k-fold cross-validation approach (where k=5).

FIGS. 28A, 28B, 28C, 28D, 28E, 28F, 28G, 28H, 28I, 28J, 28K, 28L illustrate training and testing result comparisons of different deep learning models (WestRo Porti dataset) for the k-fold cross-validation (k=5). For example, the training/testing accuracy (FIG. 28A), loss (FIG. 28B), and AUC (FIG. 28C) for our fractional dynamics deep learning approach are shown, where the training processes use signal signatures extracted with the fractional dynamic mathematical model. The training/testing accuracy (FIG. 28D), loss (FIG. 28E), and AUC (FIG. 28F) for the Vanilla DNN model, where the training processes use the physiological signals recorded with the Porti portable sleep monitors. The training/testing accuracy (FIG. 28G), loss (FIG. 28H), AUC (FIG. 28F) for the LSTM model are shown, where the training processes use the physiological signals. The training/testing accuracy (FIG. 28J), loss (FIG. 28K), and AUC (FIG. 28L) for the CNN model are shown, where the models are trained with physiological signals.

FIGS. 28A, 28B, 28C present our fractional-dynamics deep learning model's performance in terms of accuracy, loss, and AUC among training and testing datasets, respectively. FIG. 28A shows that both training and testing accuracy curves present an increasing trend—suggesting that our model improves the prediction accuracy over time without overfitting—to a predicting accuracy of 96.18%±0.48%. FIG. 28B illustrates that with increasing of the epoch numbers, the training and testing AUC curves also converge to optimal steady states. FIG. 28C shows that the loss curves for both training and testing processes exhibit a decreasing tendency, which indicates that, in our neural network, the value of the loss function converges over epochs.

FIGS. 28D, 28E, 28F respectively, show the training and testing accuracy, loss, and AUC curves obtained from the Vanilla DNN model trained with physiological signals (raw data). The testing results for the Vanilla DNN model show that the testing accuracy and AUC curves present a decreasing trend over epochs, which is evidence for overfitting. Consequently, we employ the early-stop mechanism to maintain the performance of the Vanilla DNN model (save the best-performance model). The best-performance model under Vanilla DNN exhibits a much lower prediction accuracy (31.23%±4.58%) than the fractional dynamics deep learning model, which illustrates that our model outperforms the Vanilla DNN model.

FIGS. 28G, 28H, 28I respectively, present the training and testing accuracy, loss, and AUC results curves for the LSTM model trained with physiological signals (raw data). Observing the training and testing result curves in FIGS. 28G, 28H, 28I, we notice that the LSTM model also overfits. To deal with this situation, we also utilize the early-stopping mechanism to choose the best-performance LSTM model. The prediction accuracy of the best-performance LSTM model yielded 81.17%±2.52%, whereas our fractional dynamics deep learning model presents a significantly higher prediction accuracy of 96.18%±0.48%.

We also investigated whether the convolutional neural network (CNN) model can outperform our fractional-dynamics deep learning model by characterizing the dynamics of the physiological signals with higher accuracy. The results are presented in FIGS. 28J, 28K, 28L. FIGS. 28J, 28K, 28L respectively, show the training and testing accuracy, loss, and AUC results for the CNN model trained with physiological signals (raw data). The results show that the CNN model correctly classified 23.77%±0.02% COPD samples under k-fold cross-validation (k=5). Thus, our fractional dynamics deep learning model predicts patients' COPD stages with a much higher accuracy than the Vanilla DNN, LSTM, and CNN models—trained with physiological signals (raw data)—without overfitting (for detailed information about network architecture, see section Methods, subsection Neural network architecture for the WestRo Porti COPD dataset). Of note, the testing accuracy of 96.18%±0.48% for the WestRo Porti COPD dataset is slightly lower than the testing accuracy for the WestRo COPD dataset. The reason is that the coupling matrix for the WestRo COPD dataset has more features than the coupling matrix for the WestRo Porti COPD dataset (i.e., 144 features vs. 36 features), and fewer features degrade the predicting accuracy.

### Pre-Patient Based K-Fold Analysis

In this section, we performed the FDDLM's k-fold cross-validation results such that training does not use data from individuals considered in testing. (Nonetheless, our hold-out validation makes this type of evaluation implicit because each patient belongs to just one institution). FIGS. 29A, 29B, and 29C presents our model's performance as accuracy, loss, and AUC curves between training and testing datasets (where k=5). Based on FIGS. 29A, 29B, and 29C, we find that our model yielded an accuracy of 98.70%±0.407% (very close to the accuracy we reported in the initially submitted manuscript).

FIGS. 29A, 29B, and 29C illustrate training and testing result comparisons of different deep learning models (WestRo Porti dataset) for the k-fold cross-validation (k=5). The training/testing accuracy (FIG. 29A), loss (FIG. 29B), and AUC (FIG. 29C) for our fractional dynamics deep learning approach, where the training processes use signal signatures extracted with the fractional dynamic mathematical model. The training/testing accuracy (d), loss (e), and AUC (f) for the Vanilla DNN model, where the training processes use the physiological signals recorded with the Porti portable sleep monitors. The training/testing accuracy (g), loss (h), AUC (f) for the LSTM model, where the training processes use the physiological signals. The training/testing accuracy (j), loss (k), and AUC (l) for the CNN model, where the models are trained with physiological signals.

FIG. 30 is a block diagram illustrating an example computing system 2100 for implementing the fractional dynamics foster deep learning of COPD stage prediction. The computing system 2100 can include a processor 2110, a memory 2120, and a communication interface 2130.

The processor 2110 can execute one or more instructions. The processor 2110 can obtain one or more of the instructions via at least one of the memory 2120 and the communication interface 2130. The processor 2110 can include an electronic processor, an integrated circuit, or any combination thereof, for example, including one or more of digital logic, analog logic, digital sensors, analog sensors, communication buses, volatile memory, nonvolatile memory, or any combination thereof. The processor 2110 can include but is not limited to, at least one microcontroller unit (MCU), microprocessor unit (MPU), central processing unit (CPU), graphics processing unit (GPU), physics processing unit (PPU), embedded controller (EC), gate array, programmable gate array (PGA), field-programmable gate array (FPGA), application-specific integrated circuit (ASIC), or any combination thereof. The processor 2110 can include a memory operable to store or storing one or more instructions for operating components of the processor 2110 and operating components operably coupled to the processor 2110. The one or more instructions can include at least one of firmware, software, hardware, operating systems, embedded operating systems, or any combination thereof. A bus can communicate one or more instructions, signals, conditions, states, or any combination thereof, for example, between one or more of the processor 2110, the memory 2120, and the communication interface 2130. The bus can include one or more channels, lines, traces, or any combination thereof, for example, that can perform digital or analog communication.

The memory 2120 can store data associated with the example computing system 2100. The memory 2120 can include a hardware memory device to store binary data, digital data, or any combination thereof. The memory 2120 can include one or more electrical components, electronic components, programmable electronic components, reprogrammable electronic components, integrated circuits, semiconductor devices, flip flops, arithmetic units, or any combination thereof. The memory 2120 can include at least one of a nonvolatile memory device, a solid-state memory device, a flash memory device, and a NAND memory device. The memory 2120 can include one or more addressable memory regions disposed on one or more physical memory arrays. For example, a physical memory array can include a NAND gate array disposed on a particular semiconductor device, integrated circuit device, printed circuit board device, or any combination thereof.

The communication interface 2130 can communicatively couple at least the processor 2110 to an external device. For example, an external device can include but is not limited to a smartphone, mobile device, wearable mobile device, tablet computer, desktop computer, laptop computer, cloud server, local server, or any combination thereof. The communication interface 2130 can communicate one or more instructions, signals, conditions, states, or any combination thereof between one or more of the processor 2110 and the external device. The communication interface 2130 can include one or more channels, lines, traces, or any combination thereof, for example, that can perform digital or analog communication. For example, the communication interface 2130 can include one or more serial or parallel communication lines among multiple communication lines of a communication interface. The communication interface 2130 can include one or more wired or wireless communication devices, systems, protocols, or interfaces. The communication interface 2130 can include one or more logical or electronic devices including but not limited to integrated circuits, logic gates, flip flops, gate arrays, programmable gate arrays, or any combination thereof. The communication interface 2130 can include one or more telecommunication devices including but not limited to antennas, transceivers, packetizers, wired interface ports, or any combination thereof. Any electrical, electronic, or like devices, or components associated with the communication interface 2130 can also be associated with, integrated with, supplemented by or complemented by the processor 2110 or any component thereof.

The computing system 2100 (e.g., the processor 2110 and the memory 2120) can be used to train AI (e.g., a DNN, CNN, or FDDLM) in the manner described. The computing system 2100 (e.g., the processor 2110 and the memory 2120) can be used to implement the AI to identify a COPD stage in the manner described herein. In some examples, a first computing system can train the AI, and a second, different computing system running the AI can identify the COPD stage. For example, the computing system 2100 can use geometric data characteristics to extract the fractional dynamics signatures with relevance for COPD, and exploit the fractional network dynamics as essential features to train a deep neural network that predicts COPD stages.

In some arrangements, the COPD-relevant physiological signals (e.g., respiratory rate, oxygen saturation, abdomen breathing effort) possess multifractal features in healthy individuals but not in COPD patients. The fractional dynamics specifically characterize the physiological processes involved in COPD. A Multi-Fractal Analysis (MFA) framework can calculate the fractional scaling function for nonderived signals (thorax, oxygen saturation, pulse, plethysmograph, nasal pressure, and abdomen) under different q values and find that most of these scaling functions will converge to a focus point. The multifractal features for these non-derived signals can be calculated to find that most signals gathered from healthy individuals (except the signal pulse) have multifractal properties. In contrast, most signals (except the nasal pressure signal) extracted from severe COPD patients (at stage 4) do not have multifractal features. Thus, the physiological signals relevant to COPD have distinct fractional dynamics that differentiate between healthy and severe-COPD individuals.

In some arrangements, the AI can be trained using two datasets, including raw, relevant physiological signals from COPD patients. WestRo datasets, within the field of EEG/physiological signals has a relatively large number of medical records and participants. Physiological signals data can help further medical research generate more COPD diagnosis and treatment methods and hopefully save lives.

To investigate the difference in fractional properties between healthy people and COPD patients in different COPD stages, fractional-order dynamic mathematical models are implemented to capture short-range and long-range memory characteristics from various physiological signals. The modeling approach describes the system dynamics through a matrix tuple (a, A, B, C). The coupling matrix A represents the spatial coupling (or interdependencies) among the physiological processes across time. Moreover, matrix A plays an essential role in deciphering the interdependencies and correlations between the recorded physiological signals. Thus, these interdependencies (entries of A) can indicate different physical conditions and present the fractal-dynamic signatures among different signal samples. Besides, since the physiological processes may only partially observe the complex biological systems, we also consider unknown stimuli (i.e., excitations from other unobserved processes, which we cannot probe).

A DNN model for predicting the COPD stages on suspected patients from our datasets can be trained and implemented on WestRo COPD dataset (4432 medical records from 54 patients) and WestRo Porti COPD dataset (13824 medical records from 534 patients). These cases in the WestRo COPD dataset and the deep learning models by the k-fold cross-validation and hold-out validation (the input of the DNN model are the features extracted with the fractional-order dynamic model) and show that our method for COPD stage prediction has a high prediction accuracy of 98.66%±0.45%.

To evaluate the models' performance and robustness, the transfer learning mechanism is applied to investigate the generalizability of our fractional dynamics deep learning model by involving the WestRo Porti COPD datasets to test (in the WestRo Porti COPD datasets, the medical signal records are interfered by sleep apnea). Of note, these two datasets have no overlapping patients. The predicting accuracy on the test dataset is 90.13% 0.89% with fine-tuning. If the WestRo COPD datasets is involved as a test set and evaluate them with the model well-trained by the WestRo Porti COPD dataset, the prediction accuracy is 94.01%±0.61% without fine-tuning. COPD is often a silent and late-diagnosed disease, affecting over 300 million people worldwide; intrinsically, its early discovery and treatment are crucial for the patient's quality of life and—ultimately—survival. The COPD onset is a phase of early COPD (early is a concept related to time or age) where the disease may express itself with some symptoms and has a minimal airflow limitation. In this phase, the conventional COPD detection approach (i.e., spirometry) is insufficient to attain a reliable diagnosis, which calls for new COPD detection tools. Therefore, our research represents an alternative, precise diagnostic approach to overcome the limitations of the conventional (spirometry-based) method. Our work's overarching contribution is a precise COPD stages prediction before the disease onset, especially relevant for primary care, where most patients with early and mild COPD are diagnosed and treated.

Sleep apnea, a chronic disorder characterized by frequent breathing pauses, intermittent hypoxia and autonomic activation during sleep, constitutes a significant public health concern due to its potential to cause severe cardiovascular, metabolic, neurological, and respiratory health complications. Although nocturnal polysomnography (PSG) represents the gold standard for diagnosis, it is inherently laborious, costly, and intricate. While recently deep learning approaches have shown promising outcomes in sleep apnea diagnosis by employing convolutional neural networks (CNN) and long short-term memory (LSTM) models, the existing models exhibit limitations such as diminished robustness (i.e., training on relatively small datasets) or predicting only normal versus apnea without discerning apnea stages. Our research introduces a novel methodology that exploits the long-range interdependencies aware (LRIA) signal analysis to process PSG physiological signals and predicts sleep apnea severity levels.

To this end, we employed data from 512 patients across three sleep laboratories. LRIA analysis extracts the spatial coupling among physiological processes over time for each PSG recording while accounting for their long range memory characteristics; it exploits them as features for a deep neural network investigation. This innovative approach offers practical advantages over previous models, including enhanced encoding of apnea physiological signals, the capacity to predict apnea stages, and generalizability demonstrated through training on a more extensive dataset (512 patients).

Moreover, the LRIA-based technique excels in promptly estimating potential apnea stages from recorded PSG signals within ten minutes. This ground-breaking approach provides rapid, preliminary sleep apnea evaluations, adeptly processing both wake- and sleep-time signals, reinforcing its promise in revolutionizing sleep disorder diagnostics. Such features suggest that the present disclosure is more flexible than existing solutions and can adapt robustly to clinical environments and large-scale population screening.

### Introduction

Sleep apnea is a chronic disorder consisting of repeated apneas or hypopneas during sleep. An important consideration regarding sleep apnea is its significant prevalence, with some researchers suggesting the existence of a sleep apnea epidemic. Globally, it affects nearly one billion individuals, and in some countries, the prevalence of sleep apnea can exceed 50%, as illustrated in FIG. 31A. Specifically, studies conducted in North America indicate a sleep apnea prevalence of up to 30% in males and 15% in females. Similarly, another populational study in Lausanne, Switzerland indicated a moderate-to-severe apnea prevalence of 49% in male and 23% in female individuals. These findings highlight the urgent need to address sleep apnea as a significant public health issue. Undoubtedly, sleep apnea poses a serious threat to public health, as it can worsen or even trigger severe cardiovascular, metabolic, neurological, and respiratory health problems. This health condition can be treated with continuous positive airway pressure (CPAP) devices once diagnosed. If undiagnosed and left untreated, sleep apnea causes oxygen deprivation over long periods, which generates daylight sleepiness, degraded quality of life, and an increased risk of (mostly traffic) accidents. Crucially, moderate and severe sleep apnea is associated with significant health issues, including cardiovascular diseases, mental diseases, and sudden death. Furthermore, recent research uncovered that sleep apnea is an independent risk factor for COVID-19. Consequently, the early detection of sleep apnea through screening in a large population becomes paramount in order to mitigate such issues.

The diagnostic process for sleep apnea contains three elements; the clinical interview, the physical examination and the sleep diagnostic test. An existing method for the sleep apnea diagnostic test is the whole-night polysomnography (PSG), a technique that uses sensors to collect various physiological signals simultaneously9, including electroencephalogram (EEG), electrooculogram (EOG), electromyogram (EMG), electrocardiogram (ECG), oronasal airflow, ribcage movements, abdomen movements, and oxygen saturation. Although full-night polysomnography (PSG) is widely considered thorough, accurate, and reliable, its shortcomings prevent it from being a feasible population screening method. Indeed, PSG is rather time-consuming, expensive, and complicated, requiring trained sleep technologists to monitor and diagnose sleep apnea events; at best, this situation produces long lists of people waiting for a diagnosis; at worse, it excludes many populations worldwide from being diagnosed.

Consequently, such healthcare system issues call for an autonomous method that accurately diagnoses sleep apnea events with a lower cost and a more straightforward process. In following the demand for an autonomous, computer-based method for sleep apnea diagnosis, deep learning has shown higher performance over conventional machine learning models in recent years. Accordingly, the convolutional neural network (CNN) and long short-term memory (LSTM) classifiers are the most frequently used. Six deep learning-based algorithms can be used in the automatic detection of sleep apnea from a single-lead ECG signal, including DNN, one-dimensional CNN, two-dimensional CNN, recurrent neural networks (RNN), LSTM, and gated-recurrent unit (GRU). Additionally, a model called the multi-resolution residual network (Mr-ResNet), which is based on a residual network, can be used to automatically detect nasal pressure airflow signals recorded by polysomnography (PSG), with sensitivity, specificity, accuracy, and an F1-score of 90.8%, 90.5%, 91.2%, and 90.5%, respectively. In order to take full advantage of Mr-ResNet's ability of feature extraction and encoding the signals information into compressed latent space, it can be used with a short-time Fourier transform (STFT) to convert the original time series data into spectrograms.

However, given that STFT is not explicitly designed for the PSG signal data, it may not be able to preserve the maximum amount of original, useful information. Moreover, a normalized ECG signal can be used with a first plurality of subjects for training and a second plurality of subjects for testing, which can be used to form an LSTM and a GRU using six RNN layers.

Previous studies are associated with several limitations: First, most deep learning (DL) models have been trained on a small number of participants (specifically, fewer than 80), resulting in poor generalization; these models often perform inadequately on other cohorts as they are overly fitted to the limited data set. Second, some DL models only focus on binary outcomes ‘apnea’ or ‘normal/no-apnea’ instead of categorizing the apnea stages (ranging from stage 0 to stage 3). Third, these studies lack an effective encoding approach for processing raw signals, particularly electroencephalography (EEG) signals, to extract meaningful features. In this work, the original contribution lies in applying LRIA analysis to process polysomnography (PSG) physiological signals for the first time. This novel method addresses the limitations of previous approaches by enhancing the deep learning prediction of sleep apnea severity levels. For this purpose, we collected mobile and hospital PSG data from 512 patients between 2013 and 2021 in three sleep laboratories: Victor Babes Hospital, CardioPrevent, and MediCover, located in Timisoara, Western Romania. These data were also contributed to the ESADA database. To ensure the broadest possible relevance of our findings, we retained only those physiological signals that were common to both the mobile and hospital PSG devices used in our study.

The present disclosure, which combines LRIA with deep learning for predicting apnea severity stages, offers innovative features and several advantages over previous approaches, including, for example: A novel LRIA model that distills apnea physiological signals into a condensed latent space through coupling matrices, enhancing the depiction of apnea-related patterns; a deep neural network (DNN) that leverages these matrices to predict apnea stages with precision, enabling the classification of apnea severity beyond binary categorization; allowing the DNN to operate with just ten minutes of data input, marking a significant advancement over conventional methods that necessitate overnight studies, and thus increasing efficiency without compromising predictive accuracy; and validating the LRIA model's efficiency and adaptability with robust training and testing on a diverse 512-patient dataset, ensuring its capacity to predict apnea stages across varied populations, thereby bolstering real-world applicability.

### Results

We employed LRIA analysis 3150C frameworks to extract short- and long-term memory, utilizing the compressed features stored within the coupling matrices 3160C. These features were then used to train a fractional-based deep learning framework designed to analyze physiological signals. These signals 31101B, 3110C, 3130C were procured from 10-minute segments of overnight time-series data, collected using data recording devices 3120B with participants 3130B, (refer to architecture diagram 3100B and, more generally, FIG. 31B). To validate the embodiments of a deep neural network (DNN) model disclosed herein, we implemented a k-fold cross-validation approach 3100C. In this case, our dataset was divided equally into five distinct subsets. Each subset was, in turn, selected as the test set, while the remaining subsets were designated as the discovery sets for training our DNN framework.

Since the existing models performed poorly in analyzing and extracting LRM from very-long medical physiological signals, in this work, we involved a fractional dynamic model for extracting and selecting the inner correlations from the raw data. The fractional dynamics will group and reorganize fractional features into a feature map (see FIG. 31C).

As depicted in FIGS. 31C-D, our deep learning architecture 3100D comprises a total of 12 layers, including 4 dense layers (3160D), 4 batch normalization layers, 3 dropout layers (3130D), and 1 output layer (3140D, 3170D). The implementation of the DNN framework was done using Python with the Tensorflow package, and the execution took place on a server equipped with an Nvidia Tesla V100 32 G GPU. The input layer 3120D consists of 256 neurons, and the subsequent three hidden layers have 256, 128, and 128 neurons, respectively. Following the first three fully connected layers, a dropout layer 3130D with a 20% drop rate is employed to mitigate overfitting. Additionally, batch normalization layers are added below each fully connected layer to aid in the normalization of the input values 3110D. The activation function used for each fully connected hidden layer is the Rectified Linear Unit (ReLU), which returns the input value when it is ≤0 and returns 0 otherwise (g(z)=max(0, z) for an input value z). The output layer 3170D consists of 4 neurons, and the activation function used is softmax, which normalizes the input values into a probability distribution. For optimization, we chose the Adam optimizer with a learning rate of 0.001. The loss function utilized is categorical cross-entropy.

We followed standard methods for hyperparameter tuning in deep neural networks, ensuring that the hyperparameters of all baseline models were tuned using the same procedure for a fair comparison. The following classical machine learning classifiers are involved in making the comparison: DNN, CNN, CNN-LSTM, and short-time Fourier transform-CNN (STFT-CNN). STFT-CNN is one of the state-of-the-art architectures for detecting sleep apnea, where STFT segmented the long temporal signal into subsequences with identical lengths and use Fourier Transform to compute each short segment. Furthermore, the CNN-LSTM model is also the latest model for analyzing apnea-ECG signals. The rationale for designing this architecture is as follows: (i) CNN can efficiently extract features from physiological signals; (ii) LSTM can capture the internal memory and utilize feedback connections to learn temporal information from input signals. The models' prediction results were evaluated based on accuracy, precision, recall, F1 score, and area under the receiver operating characteristic (AUROC).

We conducted evaluation using k-fold cross-validation with k=5. The performance of different models in terms of accuracy, precision, recall, F1 score, and AUROC is illustrated in FIG. 32 (the histograms show the mean value among all k experiments). We trained and tuned the hyperparameters of all the machine learning models using the same set of medical records, and we get the test result by testing models on the same independent test cohort (the only difference is the input data, where the LRIA model' coupling matrices as input features whereas the baseline models use the raw signals). To ensure fairness, all models shared the same optimization function, loss function, training, and testing processes. From the results presented in FIG. 32, we observe that our LRIA model outperforms DNN, CNN, CNN-LSTM, and STFT-CNN models. This indicates that the LRIA model is capable of accurately extracting relevant features from physiological signals and predicting patients' sleep apnea stages (3150D, as shown in FIG. 31D). It is worth noting that the STFT-CNN and CNN-LSTM models did not perform well in analyzing the physiological signals. This can be attributed to two reasons: firstly, these models struggle to capture long-range inner correlations in time-series data, and secondly, previous works trained their models on medical cases from only 10-40 patients, which limits their generalizability to larger cohorts.

To visually illustrate and explain the prediction performance of all models, we present the data visualization results for each model in FIG. 33, (a) displays the distribution of samples from the test set across all apnea stages. The test set corresponds to the fifth fold, while the first four folds are used for training the model. Panels (b-f) exhibit the t-SNE two-dimensional results for the LRIA model, DNN, CNN, STFT-CNN, and CNN-LSTM, respectively. Different colors represent the actual sleep apnea stages. A round shape denotes correct predictions by the model, while an “x” mark signifies misclassified samples. In t-SNE, if clusters of the same category have distinct boundaries, deep learning models can easily learn and differentiate these patterns. However, when clusters overlap, it becomes more challenging for the models, resulting in increased errors within these regions. Upon observing the t-SNE results generated by the LRIA model, we observe that stage 0 samples (representing healthy patients) are gathered into distinct clusters that are separate from the remaining stages (representing apnea patients).

This suggests that our LRIA model successfully drew a clear boundary between healthy and unhealthy samples. Conversely, in the t-SNE results of the DNN, CNN, STFT-CNN, and CNN-LSTM models, while clusters tend to form for samples within the same categories, most clusters lack clear boundaries, and numerous clusters from different labels overlap with each other, which adversely affects the models' performance.

FIG. 34 presents the confusion matrices obtained for the LRIA model, DNN, CNN, STFT-CNN, and CNN-LSTM models, respectively, using the fifth fold as the test set. The results shown in FIG. 34 demonstrate that LRIA model consistently outperforms all the baseline models in terms of prediction accuracy throughout the hold-out validation process. Specifically, when distinguishing between stage 0 (healthy participants) and stages 1-3 (sleep apnea patients), LRIA model achieves a remarkable performance. Only 4% of healthy participants are misclassified as having apnea, while all patients with sleep apnea are correctly identified. This characteristic of the model holds great potential for reducing the likelihood of misdiagnosis.

### Discussion

Predictive tools have the potential to greatly enhance the detection rate of Obstructive Sleep Apnea (OSA), even among patients not exhibiting symptoms. In the United States, approximately 80% of the nearly 30 million adults with OSA remain undiagnosed. This leads to an estimated $149 billion spent annually on healthcare costs, lost work productivity, and the ramifications of workplace and motor vehicle accidents. The US Preventive Services Task Force has concluded, however, that current evidence is insufficient to evaluate the balance of benefits and harms of screening for OSA in the general adult population.

In both clinical and community-based samples, the symptom-free artificial neural network tool exhibits a diagnostic performance similar to that of STOP-BANG, a tool that incorporates patient symptoms into its analysis. Algorithms derived from machine learning have shown promise for widespread identification of OSA. Future avenues of research include capturing clinically meaningful OSA endophenotypes through the use of genetics, blood biomarkers, machine/deep learning, and wearable technologies. A variety of classic machine learning methods have been evaluated for this purpose.

Currently, research studies have demonstrated that deep learning models can be utilized to analyze patterns in physiological signals, specifically ECG, for predicting the sleep apnea stages of participants. Rather than simply attempting to replicate human scoring, deep learning approaches may be more effectively utilized in examining and seeking to improve the diagnostic criteria. This approach would allow for a more nuanced understanding of the intricate circumstances surrounding OSA. However, relying solely on deep learning models may not yield accurate predictions for sleep apnea stages across different cohorts. This can be attributed to two main factors. Firstly, existing well-known deep learning architectures, such as LSTM and Transformer, are not efficient in extracting meaningful features from very long time series data. Secondly, the most predictive features in this context are complex features that require advanced mathematical and computational techniques to analyze. For example, it is well-established that patterns in heart rate exhibit strong correlations with sleep cycles. These predictive cardiac features encompass complex characteristics like spectral band power ratios and variability measures. Therefore, recognizing the constraints posed by these limitations, we have introduced an LRIA signal analysis approach in our study. Instead of directly feeding raw physiological signals into the DL models, we employ a fractional dynamic model to examine the signal geometry and extract high-order features, known as the coupling matrix, from the raw data. By incorporating these preprocessed features into a conventional DNN architecture, our proposed approach demonstrates superior performance in accurately predicting sleep apnea stages across all four stages, surpassing all the baseline models (DNN, CNN, STFT-CNN, CNN-LSTM). It is particularly effective in identifying healthy individuals, and it accurately predicts mild (82%), moderate (82%), and severe (72%) OSA. This characteristic has the potential to significantly decrease the likelihood of misdiagnosis Recent studies have highlighted the significant impact of obstructive sleep apnea on overall health outcomes. It has been established that treating this condition can lead to improved quality of life and minimized adverse clinical outcomes. Therefore, placing emphasis on effective obstructive sleep apnea treatment presents an opportunity to reduce associated healthcare costs and mitigate the negative consequences of the condition, such as cognitive impairment due to sleepiness, including the risk of accidents. However, diagnosing and treating sleep disorders can be financially burdensome, and some health economic studies have indicated that managing obstructive sleep apnea can not only be cost-effective but also potentially cost saving by preventing major complications. Consequently, individuals with lower socioeconomic status may face challenges in accessing clinics for sleep disorder prevention and diagnosis. In our research, we have developed the LRIA method, which is simple, robust, and requires only a relatively short duration of physiological signal recordings (10 minutes) to make a stable diagnosis. Therefore, our approach provides equal opportunities for individuals to access reliable medical consultations for sleep disorders, regardless of their socioeconomic status.

### Methods

According to the American Academy of Sleep Medicine (AASM), a breathing pause or apnea is defined as a drop in respiratory airflow or thoracoabdominal motion of at least ≥90% for a duration of at least ten seconds. Hypopnea is a ≥30% drop in respiratory airflow for more than ten seconds, associated with a ≥3% reduction in oxygen desaturation or arousal. Apneas and hypopneas can be categorized as obstructive, central, or mixed events based on the underlying cause related to the anatomical and neurochemical control of the upper respiratory tract or respiratory musculature. In central events, the brain reduces stems respiratory motor outputs, whereas in obstructive events, the partial or complete upper airway collapse causes the reduction or cessation of airflow; mixed events are a mixture of central and obstructive events 37. The sleep apnea-hypopnea index (AHI), used by healthcare professionals measures the severity of the disease, and represents the number of apneas and hypopneas per hour of sleep. A four-stage grading system was defined as an AHI of 0-5 indicates no sleep apnea, 5-15 mild, 15-30 moderate, and ≥30 severe sleep apnea, corresponding to stage zero to three. Although there is another more complicated grading system, the Baveno classification, which includes multicomponent to classify the severity of patients and may provide more accurate guidance in personalized treatment, the AHI-based system is still more prevalent in clinical practice as it is more straightforward and precise enough in most scenarios. This research used the AHI-based stage classification system to generate labels as desired output for all patients.

The patients included in this study were referred to accredited sleep labs for evaluation. Two types of polysomnography devices were used: the Alice 6 by Philips Respironics and the Porti 8. The raw data collected from these devices were manually validated by certified somnologists following the guidelines outlined in the AASM Manual for the Scoring of Sleep and Associated Events, VERSION 2.2. The selected study cohort consists of 6,402 medical records obtained from 342 patients across three clinics in Western Romanian. The distribution of sleep apnea stages within this cohort is as follows: 57.02% severe, 27.19% moderate, 12.57% mild, and 3.22% normal individuals. The medical records encompassed long-term physiological signals, anthropometric data, medical history, and demographic information. For a visual representation of the demographic information, please refer to FIG. 35. Additionally, FIGS. 36A-B provide a comprehensive display of the correlations among various features, ensuring the realism and consistency of the data.

The present disclosure (e.g., the study discussed herein) leveraged Respiratory Inductance lethysmography to record six physiological signals: flow, O2, pulsewave, snoring, thorax, and abdomen. Existing research indicates that dynamic, complex biological systems display long-range memory (LRM) properties, which can significantly benefit from cyber-physical systems (CPS)-oriented approaches. For example, various recent studies have shown that phenomena such as stem cell division times, blood glucose dynamics, heart rate variability, and brain-muscle interdependence activity44 can either adhere to power-law distributions or follow the principles of fractional statistics. In this investigation, our devices offered a total of twelve channels, from which we selected six for analysis: thorax, snoring, pulsewave, flow, SaO2, and abdomen. In the domain of deep learning, long short-term memory (LSTM) has been extensively employed to extract memory from biological signals for prediction or classification tasks, thereby capturing and analyzing LRM in complex processes. However, LSTM is not fully equipped to capture LRM in extended time-series data like EEG, as it necessitates making a decision on whether to retain or discard information from previous timestamps. When it comes to the final timestamp, the LSTM can barely “remember” the information from the initial step. The Transformer is another state-of-the-art deep learning architecture that can capture inner correlations in lengthy time-series data. However, Transformers are computationally expensive to train and perform optimally only when trained with a vast amount of data. Hence, both LSTM and Transformer are neither accurate nor efficient in analyzing very long time series data with LRM. To accurately capture both short- and long-range memory in physiological signals and predict sleep apnea stages, we employed a generalized mathematical model of LRIA analysis.

$$\begin{matrix}{Δ}^{α} x [ k + 1 ] = Ax [ k ] + Bu [ k ]\end{matrix} \qquad EQUATION ( 1 )$$

$$y [ k ] = Cx [ k ] ,$$

where x∈Rⁿ is the state of the biological system, u∈R^{p} is the unknown input and y∈Rⁿ is the output vector. This generalized mathematical model of the raw data provides the following advantages: Firstly, it allows our method to generate both short-range and long-range memory of individual physiological signals. Specifically, the discrete nature of our dataset necessitates the differential operator, A, which is the equivalent of derivatives in continuous mathematical models, i.e., Δ^{a} x[k]=x[k]−x[k−α]. Thus, when a is set to one, we obtain the classical one-step memory of linear-time invariant models. However, to extract the long-range memory features of several signal channels, a more generalized equation is needed. This leads us to expand the LRIA derivative and discretize any i^{th} state (1≤i≤n) as

$$\begin{matrix}{Δ}^{{α}_{i}} {x}_{i} [ k ] = {\sum }_{j = 0}^{k} ψ ( {α}_{i} , j ) {x}_{i} [ k - j ] ,\end{matrix} \qquad EQUATION ( 2 )$$

where αⁱ is the LRIA order corresponding to the i^{th} state and

$$ψ ( {α}_{i} , j ) = \frac{Γ ( i - {α}_{i} )}{Γ ( - {α}_{i} ) Γ ( j + 1 )}$$

with Γ(.) denoting the gamma function. Equation (2) demonstrates the capability of the LRIA order derivative framework in capturing long-range memory by considering all xᵢ[k−j] terms.

Our LRIA approach employs mathematical modeling to characterize complex biological systems. Specifically, we use a matrix tuple (α, A, B, C) of appropriate dimensions, where the coupling matrix A signifies the spatial coupling between various physiological processes over time and the input coupling matrix B dictates how the raw signals influence these processes. By assumption, the size of the unknown input is always strictly smaller than that of the state vector, (e.g., p<n in equation (1)). One element of the tuple is the coupling matrix, A, which facilitates the representation of the LRIA correlation between physiological signals. In our work, we can leverage the LRIA correlations between different signal channels or even the correlations within the different time series of a single physiological channel. The generated A matrix can serve as a more reliable input for deep learning models to perform classifications. FIGS. 31A-D demonstrate the LRIA analysis processes.

Thirdly, the complex biological system of the human body encompasses unknown stimuli, which are excitations recorded by electrophysiological devices but not directly arising from the observed processes. Such unanticipated perturbations could introduce errors in the machine learning processes. Consequently, we further examine the role of the vector variable u and investigate the impact of these dynamics on our model. As a result, our mathematical model transforms into a comprehensive multi-dimensional fractional-order linear LRIA model with unknown unknowns, i.e., excitations from unobserved processes. The parameters of this model are optimized using the Expectation-Maximization (EM) algorithm as presented in the research by Gupta, G. et al. The cited work also demonstrates that this convergent algorithm can minimize modeling errors. Through this rigorous and comprehensive processing of the raw physiological signals, we can handle imperfect or even corrupt data samples introduced by the limitations of medical devices.

The LRIA deep learning model consists of two parts: (1) LRIA signature extraction and (2) deep learning classifier. Of note, by defining the input of the training data as the coupling matrix A compressed from the LRIA model, we constructed a deep neural network (DNN) architecture to handle the training and prediction progress. We built the network with the TensorFlow Python framework. One embodiment of our deep neural network consists of 12 layers: 4 hidden layers, 4 batch normalization layers, 3 dropout layers, and 1 output layer. Also, we resampled the input data (matrix A) to 36×1 voxels and normalized each value within the range [0, 1](normalization is a technique for training deep neural networks that standardizes the inputs to a layer). In one embodiment, we placed the dropout layers after the first three hidden layers with a 20% drop rate and placed the batch normalization layers after each hidden layers. Each fully connected hidden layer utilizes the ReLU activation function. The softmax is utilized as the activation function in the output layer. The DNN is optimized with the Adams optimizer with a learning rate of 0.0001

and trained with the cross-entropy loss function. In some embodiments, the LRIA model is trained over 800 epochs with a batch size of 64 samples. Overall, the number of trainable parameters of the deep learning model is 74,105.

FIG. 37 is an illustration of a method 2800 of identifying a stage of one or more medical records using a deep neural network, according to some embodiments. The method may be implemented by one or more processors, such as, for example the processor 2110 of the computing system 2100 shown in, and described with reference to, FIG. 30. The method 2800, however, is not limited to computing system 2100. Accordingly, in some embodiments, the method 2800 (or any portion thereof) may be performed by one or more additional and/or distinct processors, such as a remote server, cloud computing environment, among other examples.

At 2810, the method 2800 may include receiving, by a processor, a plurality of physiological signals datasets comprising one or more physiological signals. At 2812, the method 2800 may include identifying, by the processor, a first physiological signal of the physiological signals datasets. At 2814, the method 2800 may include analyzing, by the processor, the first physiological signal of the physiological signals datasets. In some examples, at 2816, the method 2800 can include extracting, by the processor, one or more fractional dynamics signatures specific to one or more corresponding medical records. In some embodiments, the one or more corresponding medical records can include a sleep apnea condition. In some embodiments, the one or more corresponding medical records can include a chronic obstructive pulmonary disease (COPD) condition. In some examples, at 2818 the method 2800 can include identifying, using a deep neural network, a corresponding stage

of the one or more medical records.

Additional disclosure is attached hereto as Exhibit A, the entire content of which is incorporated herein by reference in its entirety.

Throughout and within this disclosure various technical and patent literature are referenced with a bibliographic citation or a reference to a citation that may be found immediately preceding the claims. The disclosures of the technical and patent literature are hereby incorporated by reference into the present disclosure in their entireties.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

As used in the specification and claims, the singular form “a”, “an” and “the” include plural references unless the context clearly dictates otherwise. For example, the term “a polypeptide” includes a plurality of polypeptides, including mixtures thereof.

A “subject” of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporids, such as rabbits, livestock, sport animals, and pets.

As used herein, the terms “treating,” “treatment,” and the like are used herein to mean obtaining a desired pharmacologic and/or physiologic effect. The effect may be prophylactic in terms of completely or partially preventing a disease, disorder, or condition or sign or symptom thereof, and/or may be therapeutic in terms of a partial or complete cure for a disorder and/or adverse effect attributable to the disorder. In one aspect, treatment is a reduction in tumor burden, a reduction in tumor size, remission or an inhibition of metastatic potential or metastasis of the tumor. In aspect, the term excludes a prophylactic effect or prevention of cancer.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

What is claimed is:

1. A method, comprising:
receiving, by a processor, a plurality of physiological signals datasets comprising one or more physiological signals;
identifying, by the processor, a first physiological signal of the physiological signals datasets;
analyzing, by the processor, the first physiological signal of the physiological signals datasets;
extracting, by the processor, one or more fractional dynamics signatures specific to one or more corresponding medical records; and
identifying, using a deep neural network (DNN), a corresponding stage of the one or more medical records.

2. The method of claim 1, wherein the plurality of physiological signals datasets comprises a WestRo chronic obstructive pulmonary disease (COPD) dataset and a WestRo Porti COPD dataset and the one or more fraction dynamics signatures correspond to a COPD medical record.

3. The method of claim 1, wherein the plurality of physiological signals datasets comprises a sleep apnea dataset and the one or more fractional dynamics signatures correspond to a sleep apnea medical record.

4. The method of claim 1, further comprising training the DNN to identify one of a plurality of stages of the one or more medical records using fractal dynamic network signatures.

5. The method of claim 1, comprising:
constructing a fractional dynamics deep learning model (FDDLM);
training the FDDLM using a training set to recognize COPD level based on signal signatures; and
testing the FDDLM using a test set to predict one of a plurality of COPD stages, wherein the DNN comprises the FDDLM.

6. The method of claim 1, comprising:
performing fractional-order dynamical modeling; and
extracting distinguishing signatures from the physiological signals across patients with all COPD stages.

7. The method of claim 1, wherein the COPD stage is identified using at least one of thorax breathing effort, respiratory rate, or oxygen saturation levels.

8. The method of claim 1, further comprising:
constructing a fractional dynamics deep learning model (FDDLM);
training the FDDLM using a training set to recognize sleep apnea stage based on signal signatures; and
testing the FDDLM using a test set to predict one of a plurality of sleep apnea stages, wherein the DNN comprises the FDDLM. the input of the DNN comprises features extracted from a fractional-order dynamic model.

9. A system, comprising:
a memory;
a processor, configured to:
receive a plurality of physiological signals datasets comprising one or more physiological signals;
identify a first physiological signal of the physiological signals datasets;
analyze the first physiological signal of the physiological signals datasets;
extract one or more fractional dynamics signatures specific to one or more corresponding medical records; and
identify, using a deep neural network (DNN), a corresponding stage of the one or more medical records.

10. The system of claim 9, wherein the physiological signals datasets comprises a WestRo COPD dataset and a WestRo Porti COPD dataset and the one or more fraction dynamics signatures correspond to a COPD medical record.

11. The system of claim 9, wherein the plurality of physiological signals datasets comprises a sleep apnea dataset and the one or more fractional dynamics signatures correspond to a sleep apnea medical record.

12. The system of claim 10, the processor configured to train the DNN to identify one of a plurality of COPD stages using fractal dynamic network signatures and expert analysis.

13. The system of claim 9, the processor configured to:
construct a fractional dynamics deep learning model (FDDLM);
train the FDDLM using a training set to recognize COPD level based on signal signatures; and
test the FDDLM using a test set to predict one of a plurality of COPD stages, wherein the DNN comprises the FDDLM.

14. The system of claim 9, the processor configured to:
perform fractional-order dynamical modeling; and
extract distinguishing signatures from the physiological signals across patients with all COPD stages.

15. The system of claim 9, wherein the COPD stage is identified using at least one of thorax breathing effort, respiratory rate, or oxygen saturation levels.

16. The system of claim 9, the processor configured to:
construct a fractional dynamics deep learning model (FDDLM);
train the FDDLM using a training set to recognize sleep apnea stage based on signal signatures; and
test the FDDLM using a test set to predict one of a plurality of sleep apnea stages, wherein the DNN comprises the FDDLM.

17. A non-transitory processor-readable medium comprising processor-readable instructions, such that, when executed by a processor, causes the processor to:
receive a plurality of physiological signals datasets comprising one or more physiological signals;
identify a first physiological signal of the physiological signals datasets;
analyze the first physiological signal of the physiological signals datasets;
extract one or more fractional dynamics signatures specific to one or more corresponding medical records; and
identify, using a deep neural network (DNN), a corresponding stage of the one or more medical records.

18. The non-transitory processor-readable medium of claim 18, wherein the processor is caused to train the DNN to identify one of a plurality of COPD stages using fractal dynamic network signatures.

19. The non-transitory processor-readable medium of claim 18, the processor is caused to:
construct a fractional dynamics deep learning model (FDDLM);
train the FDDLM using a training set to recognize COPD level based on signal signatures; and
test the FDDLM using a test set to predict one of a plurality of COPD stages, wherein the DNN comprises the FDDLM.

20. The non-transitory processor-readable medium of claim 18, the processor is caused to:
perform fractional-order dynamical modeling; and
extract distinguishing signatures from the physiological signals across patients with all COPD stages.
